(19) United States
(12) Patent Application Publication
(10) Pub. No.: US 2018/0169275 A1
(43) Pub. Date: Jun. 21, 2018

(54) POLYMERIC MICELLAR NANOPARTICLES

(71) Applicant: THE REGENTS OF THE UNIVERSITY OF CALIFORNIA, Oakland, CA (US)

(72) Inventors: Nathan GIANNESCHI, La Jolla, CA (US); Yiwen LI, La Jolla, CA (US); Yuran HUANG, La Jolla, CA (US); Miriam SCADENG, La Jolla, CA (US)

(21) Appl. No.: 15/738,260

(22) PCT Filed: Jun. 22, 2016
(86) PCT No.: PCT/US16/38786

§ 371 Date: Dec. 20, 2017

Related U.S. Application Data
(60) Provisional application No. 62/182,876, filed on Jun. 22, 2015.

(51) Int. Cl. A61K 49/18; C08G 81/02; C08F 16/10; C08F 26/06

(52) U.S. Cl. CPC A61K 49/1854; A61K 49/1809; C08K 2003/0856; C08F 16/10; C08F 26/06; C08G 81/024

(57) ABSTRACT

Described herein, inter alia, are compositions and methods for using polymeric micellar nanoparticles for nuclear magnetic resonance imaging in a subject in need thereof.

### CROSS-REFERENCES TO RELATED APPLICATIONS

This application claims the benefit of U.S. Provisional Application No. 62/182,876, filed Jun. 22, 2015, which is incorporated herein in its entirety and for all purposes.

### STATEMENT AS TO RIGHTS TO INVENTIONS MADE UNDER FEDERALLY SPONSORED RESEARCH AND DEVELOPMENT

This invention was made with government support under grant numbers RO1EB011633 and DP20D008724 awarded by the National Institutes of Health. The Government has certain rights in the invention.

### BACKGROUND OF THE INVENTION

Currently, FDA approved magnetic resonance imaging (MRI) contrast agents typically contain heavy metals such as gadolinium (Gd). The slow clearance of Gd-based chelates, however, may induce several severe diseases (e.g., nephrogenic systemic fibrosis). Accordingly, there is a need for alternative MRI contrast agents that, for example, do not include Gd, possess good r₁, low r₂/r₁ ratio, superior stability in blood serum, and sufficient in vitro MRI performance in cells. Disclosed herein are solutions to these or other problems in the art.

### BRIEF SUMMARY OF THE INVENTION

In a first aspect, there is provided a polymeric micellar nanoparticle, where the polymeric micellar nanoparticle includes a Fe(III)-catecholate complex.

In another aspect, there is provided a method of providing visibility of an internal body structure of a subject undergoing MRI by administering to the subject an effective amount of the polymeric micellar nanoparticle described herein.

In an aspect is provided a method of detecting an internal body structure of a subject, the method including administering to the subject an effective amount of the polymeric micellar nanoparticle as described herein and detecting the polymeric micellar nanoparticle using magnetic resonance imaging thereby detecting the internal body structure.

In another aspect, there is provided a method for synthesizing a polymeric micellar nanoparticle. The method includes functionalizing a block copolymer amphiphile to incorporate a catechol group in one block of the block copolymer amphiphile, thereby providing a functionalized block copolymer amphiphile. The method further includes contacting the functionalized block copolymer amphiphile under conditions suitable to afford a catechol-containing block copolymer. The method further includes contacting the catechol-containing block copolymer with a metal salt, thereby providing the polymeric micellar nanoparticle.

In another aspect, there is provided a diagnostic pharmaceutical composition including a polymeric micellar nanoparticle as set forth herein, in combination with a pharmaceutically acceptable excipient.

In another aspect, there is provided a kit including an instruction and a polymeric micellar nanoparticle described herein.

### BRIEF DESCRIPTION OF THE DRAWINGS

FIGS. 1A-1G. FIG. 1A: General synthetic scheme for amphiphilic tri-block copolymers. (a) Polymer 1: m=38, n=34, p=50. Polymer 2: m=20, n=23, p=43; FIGS. 1B-1D: Electron microscopy of SMN formed from Polymer 1: (FIG. 1B) cryo-TEM; (FIG. 1C) BF-STEM; (FIG. 1D) HAADF-STEM. (FIGS. 1E-1G) Electron microscopy of CMN formed from Polymer 2: (FIG. 1E) cryo-TEM; (FIG. 1F) BF-STEM; (FIG. 1G) HAADF-STEM.

FIG. 2. ¹H NMRD profiles for SMN and CMN. x-axis: Proton Larmor Frequency (MHz); y-axis: r₁ₚ (mM⁻¹s⁻¹). Legend: CMN (boxes); SMN (diamonds).

FIGS. 3A-3C. MRI characterization of micellar nanoparticles. FIG. 3A: Plots of 1/T₁ vs Fe³⁺ concentration for SMN in different medium with calculated r₁ (blue plot: SMN in water, red plot: SMN in FBS for 0 day, and green plot: SMN in FBS for 3 days). FIG. 3B: Plots of 1/T₁ vs Fe³⁺ concentration for CMN in different medium with calculated r₁ (blue plot: CMN in water, red plot: CMN in FBS for 0 day, and green plot: CMN in FBS for 3 days). FIG. 3C: T₁-weighted MR images from free Fe³⁺, SMN and CMN in different medium with respect to water and FBS, respectively ([Fe³⁺] is about 0.6 mM in each tube).

FIGS. 4A-4B. Magnetization data collected as a function of applied field for (FIG. 4A) SMN and (FIG. 4B) CMN from 2-300 K. Curve identification (top to bottom): 2, 4, 8, 12, 16, 20, 24, 50 and 300 K.

FIGS. 5A-5B. Stability study of Fe(III)-chelated SMN and CMN in PBS (PH=7.4) (FIG. 5A), and FBS (FIG. 5B).

FIG. 6A-6B. FIG. 6A: In vitro T₁-weighted MR images of Hela cells incubated with SMN and CMN ([Fe³⁺] is 67.5 μM) for different periods of time. FIG. 6B: Quantitative determination of intracellular Fe³⁺ content (per 10⁶ cells) for Hela cells incubated with SMN and CMN for different periods of time and their corresponded T₁ relaxation values.

FIG. 7. SEC overlays for polymers: (a) Homopolymer 1 (rightmost curve), Copolymer 1-b-2 (middle curve), and (OEG)₃₈-(NHS)₃₄-(C₆)₅₀ (leftmost curve).

FIG. 8. SEC overlays for polymers: (a) Homopolymer 1 (rightmost curve), Copolymer 1-b-2 (middle curve), and (OEG)₂₀-(NHS)₂₃-(C₆)₄₃ (leftmost curve).

FIGS. 9A-9B. ¹H NMR spectra of (FIG. 9A) (OEG)₂₀-(NHS)₂₃-(C₆)₄₃, and (FIG. 9B) (OEG)₂₀-(Cat)₂₃-(C₆)₄₃. X-axis (both FIGS. 9A and 9B: chemical shift (ppm)).

FIGS. 10A-10B. ¹H NMR spectra of (FIG. 10A) (OEG)₃₈-(NHS)₃₄-(C₆)₅₀, and (FIG. 10B) (OEG)₃₈-(Cat)₃₄-(C₆)₅₀. X-axis (both FIGS. 10A and 10B: chemical shift (ppm)).

FIGS. 11A-11B. ¹³C NMR spectra of (FIG. 11A) (OEG)₂₀-(NHS)₂₃-(C₆)₄₃, and (FIG. 11B) (OEG)₂₀-(Cat)₂₃-(C₆)₄₃. X-axis (both FIGS. 11A and 11B: chemical shift (ppm)).

FIGS. 12A-12B. ¹³C NMR spectra of (FIG. 12A) (OEG)₃₈-(NHS)₃₄-(C₆)₅₀, and (FIG. 12B) (OEG)₃₈-(Cat)₃₄-(C₆)₅₀. X-axis (both FIGS. 12A and 12B: chemical shift (ppm)).

FIGS. 13A-13B. No staining TEM images of freshly prepared micelles: (FIG. 13A) SMN, and (FIG. 13B) CMN.

FIGS. 14A-14B. FIG. 14A: Selected area BF-STEM of SMN with area chosen for EDS analysis; insert is the EDS Fe elemental mapping image of selected area. FIG. 14B: EDS profiles of SMN from the testing area (upper curve) and background (lower curve) in FIG. 14A.

FIGS. 15A-15B. FIG. 15A: Selected area BF-STEM of CMN with area chosen for EDS analysis; insert is the EDS Fe elemental mapping image of selected area. FIG. 15B: EDS profiles of CMN from the testing area (upper curve) and background (lower curve) in FIG. 15A.

FIGS. 16A-16B. No staining TEM images of micelles after 6 months storage in room temperature: (FIG. 16A) SMN, and (FIG. 16B) CMN.

FIG. 17. Fe(catecholate)₃ Example #1 with intermolecular hydrogen-bonding to piperidinium cations and H₂O. H₂O. H₂O hydrogen atoms were not located in the data set (CSD Codes: BICSEL and CATFEP).

FIG. 18. Fe(catecholate)₃ Example #2 with intramolecular hydrogen-bonding to amide N—H groups (CSD Code FUDRUR).

FIG. 19. Fe(catecholate)₃ Example #3 with intramolecular hydrogen-bonding to amide N—H groups (CSD Code SUCDAV10).

FIG. 20. Fe(catecholate)₃ Example #4 with intramolecular hydrogen-bonding to amide N—H groups (CSD Code FERJES).

FIG. 21. Fe(catecholate)₃ Example #5 with intermolecular hydrogen-bonding to indole N—H groups (CSD Code CEZYIR). Note that in this example, intermolecular hydrogen-bonds fully support a host-guest interaction between two tris(indolemethylene)amine molecules and an [Fe(Br₄Cat)₃]³⁻ trianion.

FIGS. 22A-22B. FIG. 22A: ¹H NMRD profiles for CMN, SMN, Melanin-Fe(III), and GdDOTA. FIG. 22B: Histogram depicting per Fe^{III} r₁ₚ of CMN, SMN, Melanin-Fe(III), and GdDOTA at filed strength of 1T and 1.5T, respectively (left to right in each bin).

FIGS. 23A-23F. Plots of 1/T₁ and 1/T₂ vs Fe³⁺ concentration for SMN in water (FIG. 23A), CMN in water (FIG. 23B), SMN in FBS for 0 day (FIG. 23C), CMN in FBS for 0 day (FIG. 23D), SMN in FBS for 3 days (FIG. 23E), and CMN in FBS for 3 days (FIG. 23F) with the calculated r₁ and r₂, respectively.

FIG. 24. T₁-weighted MR images from freshly-prepared SMN and CMN and after 24 hour incubation with excess transferrin protein (13 mg/mL), respectively ([Fe³⁺] is about 0.3 mM in each tube).

FIGS. 25A-25B. Cytotoxicity assay for Hela cells incubated with various concentrations of Fe(III) Ions in SMN (FIG. 25A) and CMN (FIG. 25B).

### DETAILED DESCRIPTION OF THE INVENTION

Described herein are a new class of highly efficient and non-toxic amphiphilic block copolymers containing Fe^{III}-catecholate complexes formulated as spherical- or cylindrical-shaped micellar nanoparticles. In embodiments, these new T₁-weighted agents possess high relaxivity, low cytotoxicity, and long-term stability in biological fluids. In embodiments, relaxivities of both SMN and CMN exceed those of established gadolinium chelates and synthetic melanin-Fe^{III} complexes across a wide range of magnetic field strengths.

### I. DEFINITIONS

The following definitions are provided to facilitate understanding of certain terms used frequently herein and are not meant to limit the scope of the present disclosure. The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include di- and multivalent radicals, having the number of carbon atoms designated (i.e., C₁-C₁₀ means one to ten carbons). Alkyl is not cyclized. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds (e.g. alkene, alkyne). Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂-CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂-. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Cycloalkyl and heterocycloalkyl are non-aromatic. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A substituted 1,2-dihydroxyphenyl is a 1,2-dihydroxyphenyl substituted with a substituent in addition to the two hydroxyl groups. An unsubstituted 1,2-dihydroxyphenyl is a 1,2-dihydroxyphenyl with no substituents in addition to the two hydroxyl groups.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

The term “oxo,” as used herein, means an oxygen that is double bonded to a carbon atom.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, monophosphate (or derivatives thereof), diphosphate (or derivatives thereof), triphosphate (or derivatives thereof), in a number ranging from zero to (2 m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, monophosphate (or derivatives thereof), diphosphate (or derivatives thereof), triphosphate (or derivatives thereof), in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In embodiments, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula —T—C(O)—(CRR′)_{q}-U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B—, wherein A and B are independently —CRR′—, —O—, —NR—, —S—, —S(O)—, —S(O)₂-, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ-X′—(C″R″R′″)_{d}-, where s and d are independently integers of from 0 to 3, and X′ is —O—, —NR′—, —S—, —S(O)—, —S(O)₂-, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, monophosphate (or derivatives thereof), diphosphate (or derivatives thereof), or triphosphate (or derivatives thereof), and
(B) alkyl, heteroalkyl, cycloalkyl, heterocycloalkyl, aryl, heteroaryl, monophosphate (or derivatives thereof), diphosphate (or derivatives thereof), or triphosphate (or derivatives thereof), substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, monophosphate (or derivatives thereof), diphosphate (or derivatives thereof), or triphosphate (or derivatives thereof), and (ii) alkyl, heteroalkyl, cycloalkyl, heterocycloalkyl, aryl, heteroaryl, monophosphate (or derivatives thereof), diphosphate (or derivatives thereof), or triphosphate (or derivatives thereof), substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O)NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, monophosphate (or derivatives thereof), diphosphate (or derivatives thereof), or triphosphate (or derivatives thereof), and
      (b) alkyl, heteroalkyl, cycloalkyl, heterocycloalkyl, aryl, heteroaryl, monophosphate (or derivatives thereof), diphosphate (or derivatives thereof), or triphosphate (or derivatives thereof), substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, monophosphate (or derivatives thereof), diphosphate (or derivatives thereof), and triphosphate (or derivatives thereof).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, In embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In embodiments, the compound is a chemical species set forth in the Examples section below.

Certain complexes and compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)— or (S)— or, as (D)— or (L)—-for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)— and (S)—, or (D)— and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus, a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The terms “polypeptide,” “peptide” and “protein” are used interchangeably herein to refer to a polymer of amino acid residues, wherein the polymer may optionally be conjugated to a moiety that does not consist of amino acids (e.g., a block copolymer). The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be two compounds as described.

The term “polymeric micelle” refers to a micelle comprised of the block copolymers (e.g. surfactant molecules) as described herein. The internal portion (e.g. core) of the polymeric micelle (also referred to herein as a “block copolymer micelle”) is hydrophobic while the exterior portion (e.g. shell) is hydrophilic. In embodiments, the polymeric micelle is a nanoparticle (referred to herein as “polymeric micellar nanoparticle”). In embodiments, the polymeric micelle is a spherical nanoparticle (referred to herein as a “spherical polymeric micellar nanoparticle”). In embodiments, the polymeric micelle is a cylindrical nanoparticle (referred to herein as a “cylindrical polymeric micellar nanoparticle”). In embodiments, a polymeric micelle includes a hydrophobic core comprised of hydrophobic polymerized monomers and a hydrophilic shell comprised of hydrophilic polymerized monomers.

A “nanoparticle,” as used herein, is a particle wherein the longest diameter is less than or equal to 1000 nanometers (nm). A nanoparticle (e.g., polymeric micelle) is a particle wherein the longest diameter is less than or equal to 1000 nanometers comprising a plurality of the block copolymers. In embodiments, a nanoparticle has a shortest diameter greater than or equal to 1 nanometer (e.g., diameter from 1 to 1000 nanometers). In embodiments, the nanoparticle constructs provided herein may be an approximately spherical shape (referred to herein as a “spherical nanoparticle”). In embodiments, the nanoparticle constructs provided herein may be an approximately cylindrical shape (referred to herein as a “cylindrical nanoparticle”).

The term “block copolymer” is used in accordance with its ordinary meaning and refers to a molecule including repeating subunits (e.g., polymerizable monomers). Such block copolymers may self-assemble into a polymeric micelle such as a polymeric micellar nanoparticle. In embodiments, a block copolymer is a repeating pattern of polymers. For example, a block copolymer has the formula: —B—B—B—B—B—B-A-A-A-A-A-, where ‘B’ is a first subunit and ‘A’ is a second subunit covalently bound together. Further description for block copolymer can be found herein.

The term “polymerizable monomer” is used in accordance with its meaning in the art of polymer chemistry and refers to a compound that may covalently bind chemically to other monomer molecules (such as other polymerizable monomers that are the same or different) to form a polymer thereby forming a polymerized monomer. An example of a polymerizable monomer is a ROMP polymerizable monomer, which is a polymerizable monomer capable of binding chemically to other ROMP polymerizable monomers through a ROMP chemical reaction (ring-opening metathesis polymerization) to form a polymer. It will be understood that a polymerizable monomer may be chemically modified in the polymerization reaction to differ from the free polymerizable monomer when forming the polymerized monomer moiety. In embodiments, the ROMP polymerizable monomer includes an olefin. In embodiments, the ROMP polymerizable monomer includes a cyclic olefin. In embodiments, the ROMP polymerizable monomer includes a cyclic olefin with ring strain (e.g., norbornene or cyclopentene or derivatives thereof). In embodiments, the ROMP polymerizable monomer is attached to a polypeptide. In embodiments, the ROMP polymerizable monomer is attached to a hydrophobic moiety. In embodiments, the ROMP polymerizable monomer is or includes a substituted or unsubstituted norbornenyl.

In embodiments, a polymerizable monomer is selected from:

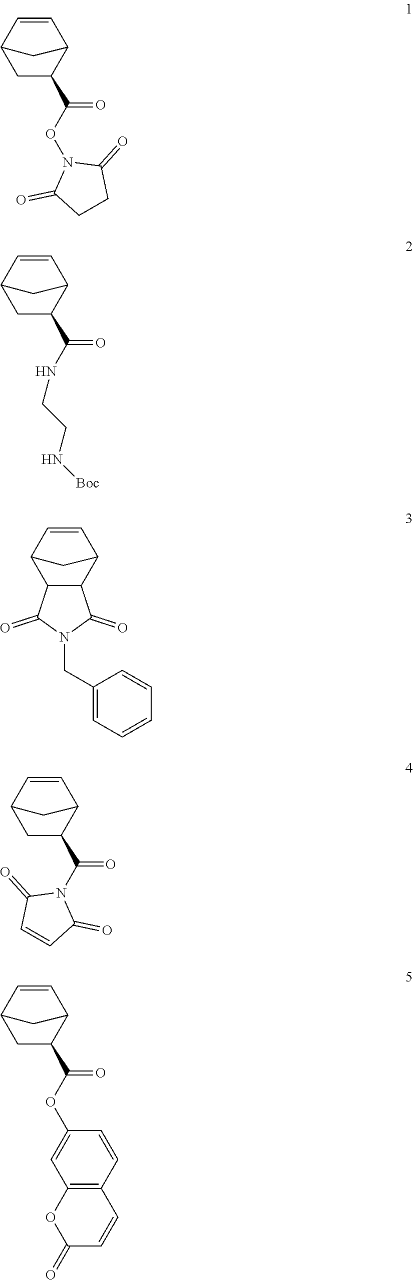

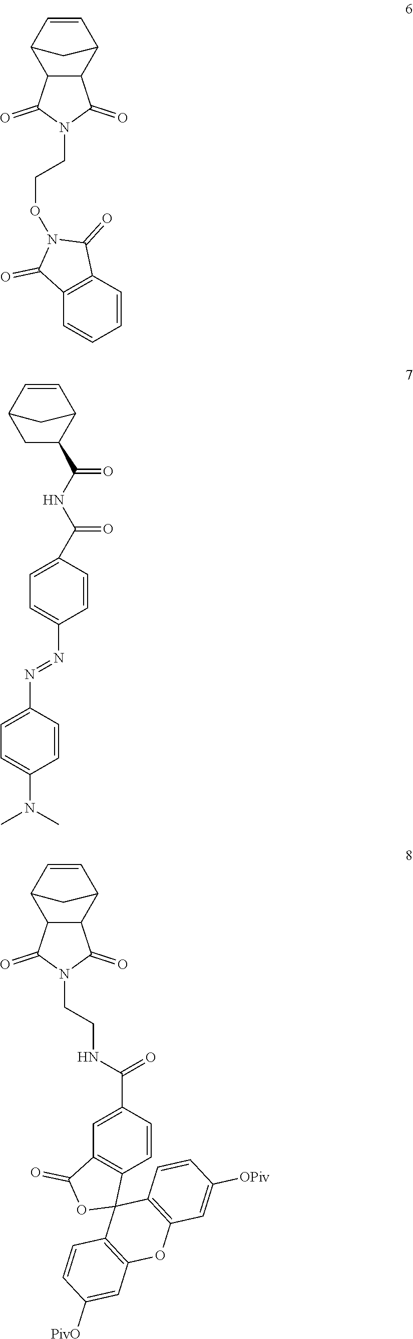

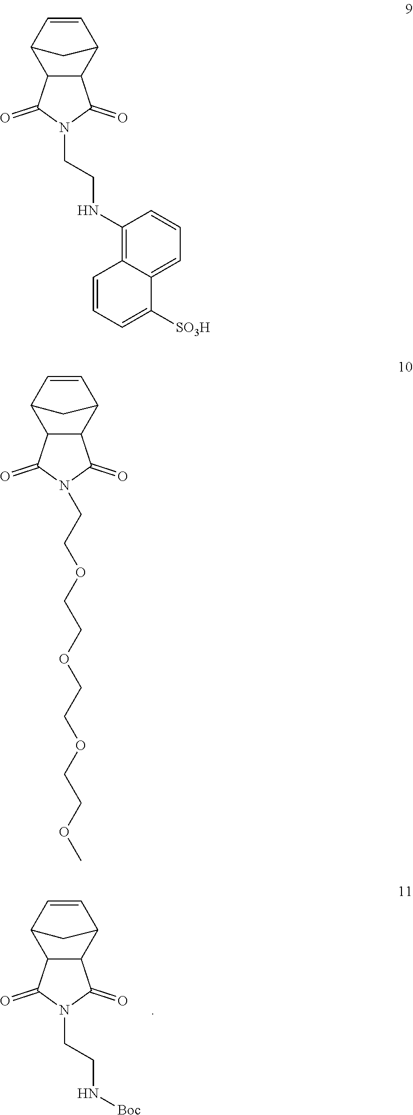

The above polymerizable monomers form the polymerized monomers within the block copolymers disclosed herein.

The term “ring-opening metathesis polymerization” or “ROMP” is used in accordance with its meaning in polymer chemistry and refers to a chain-growth polymerization (e.g., olefin metathesis chain-growth polymerization). In embodiments, the reaction is driven by relief of ring strain in cyclic olefins (e.g., norbornene or cyclopentene). In embodiments, the ROMP uses a ruthenium catalyst. In embodiments, the ROMP uses a Grubbs' catalyst. In embodiments, the ROMP uses a Mo catalyst.

In embodiments, a polymeric micellar nanoparticle contains a plurality of block copolymers including the following structure wherein the succinimide moiety may be reacted with a reactive containing a catechol group thereby forming a catechol moiety as described herein:

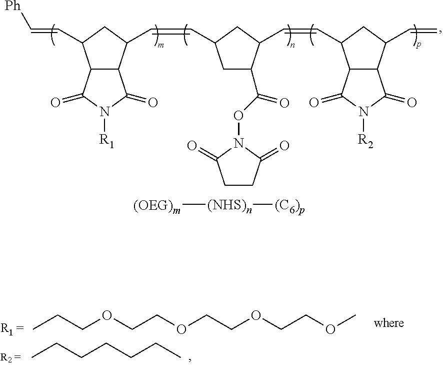

where m is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100,
where n is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100,
where p is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, and
where m, n, p are not 0 at the same time. In embodiments, m, n, and p are 15, 16, 17, 18, 19, 20, 21, 22, 23, 24 or 25. In embodiments, a polymeric micellar nanoparticle is a amphiphilic block copolymer.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, electrocardiogram, echocardiography, radio-imaging, nuclear scan, and/or stress testing, neuropsychiatric exams, and/or a psychiatric evaluation.

“Patient,” “subject” or “subject in need thereof” refers to a living organism. In embodiments, a subject refers to a living organism who is going to have an MRI scanning or who is undergoing MRI. Non-limiting examples include humans, other mammals, bovines, rats, mice, dogs, monkeys, goat, sheep, cows, deer, and other non-mammalian animals. In embodiments, a subject is human.

“Disease” or “condition” refer to a state of being or health status of a patient or subject capable of being treated with a compound, pharmaceutical composition, or method provided herein.

As used herein, the term “administering” refers to oral administration (i.e., solid or liquid), administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., cancer therapies, AIDS therapies and the like).

An “effective amount” is an amount sufficient to accomplish a stated purpose (e.g., achieve the effect for which it is administered, increase contrast in an MRI image or increase sensitivity of MRI or enhance the visibility of an internal body structure by MRI and the like). In embodiments, an effective amount is sufficient to increase contrast in an MRI image or to increase sensitivity of MRI or to enhance the visibility of an internal body structure by MRI by at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or more compared to the contrast or sensitivity or visibility level provided by similar amount of other contrast agents or compared to the contrast or sensitivity or visibility level in the absence of the contrast agent described herein. An example of an “effective amount” is an amount sufficient to contribute to the diagnosis of a symptom or symptoms of a disease, which could also be referred to as a “diagnostically effective amount.” In embodiments, the exact amounts will depend on the purpose of MRI and/or the location of the internal structure that needs MRI. The exact amounts will depend on the purpose and the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Pharmaceutically acceptable excipient,” “pharmaceutically acceptable carrier” and the like refer to a substance that aids the administration of an active agent to a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “targeting moiety” as used herein refers to a moiety that can be covalently or noncovalently attached to a compound (e.g., polymeric micellar nanoparticle) or biomolecule that serves as a recognition segment for a biological target (e.g. protein, tissue, or cell) and thereby helps in localization of the polymeric micellar nanoparticle provided herein to a target of interest. In embodiments, the targeting moiety is covalently attached. The targeting moiety may be an amino acid sequence, peptide or protein. The targeting moiety may be an antibody.

The term “drug moiety” used herein refers to a therapeutic agent that can be covalently or noncovalently attached to a compound (e.g., polymeric micellar nanoparticle) or biomolecule that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “relaxivity” is used in accordance with its ordinary meaning and refers to a characterization parameter for magnetic resonance imaging agents. Relaxivity is the degree to which the contrast agent can enhance the longitudinal or transverse water relaxation rate constant (R₁=1/T, or R₂=1/T₂, respectively) normalized to concentration of the contrast agent. Longitudinal and transverse relaxivity are denoted R₁ and R₂, respectively. In embodiments, relaxivity is a measure of the sensitivity of the contrast agent. For example, a compound with higher relaxivity provides equivalent contrast at a lower dose compared to a low relaxivity compound.

### II. COMPOSITIONS

In an aspect there is provided a polymeric micellar nanoparticle (e.g., contrast agent) for nuclear magnetic resonance imaging. In embodiments, the polymeric micellar nanoparticle includes a metal-chelator complex. In embodiments, the chelator is catecholate, carboxylate, or citrate. In embodiments, the chelator is catecholate. In embodiments, the metal is Mn²⁺, Mn³⁺, Fe³⁺, or Cu²⁺. In embodiments, the metal is Fe³⁺, alternatively written as Fe(III). In embodiments, the polymeric micellar nanoparticle includes a Fe(III)-catecholate complex. In embodiments, the metal-chelator complex includes water. In embodiments, the metal-chelator complex includes water interacting with the metal. In embodiments, the metal-chelator complex includes water interacting with the chelator.

In embodiments, the polymeric micellar nanoparticle includes a plurality of block copolymers, wherein each block copolymer includes a first block of hydrophobic polymerized monomers, a second block of catechol polymerized monomers and optionally a third block of hydrophilic polymerized monomers. The first block of hydrophobic polymerized monomers includes a hydrophobic moiety covalently attached to each first block monomer backbone moiety within the first block of hydrophobic polymerized monomers. Each hydrophobic moiety is optionally different. The second block of catechol polymerized monomers includes a catechol moiety covalently attached to each second block monomer backbone moiety within the second block of catechol polymerized monomers. At least one catechol moiety is complexed to iron thereby forming the Fe(III)-catecholate complex. The third block of hydrophilic polymerized monomers includes a hydrophilic moiety covalently attached to each third block monomer backbone moiety within the third block of hydrophilic polymerized monomers. Each hydrophilic moiety is optionally different. In embodiments, each hydrophilic moiety is identical. In embodiments, each hydrophobic moiety is identical. In embodiments, the polymeric micellar nanoparticle includes a plurality of block copolymers, wherein each block copolymer includes a first block of hydrophobic polymerized monomers, a second block of catechol polymerized monomers and a third block of hydrophilic polymerized monomers.

In embodiments, the polymeric micellar nanoparticle has the formula: R¹-L¹-(A(-L²-R²))_{z1}-(B(-L³-R³))_{z2}-[(C(-L⁴-R⁴))_{z3}]_{z5}-L⁵-R⁵. (A(-L²-R²))_{z1} is the first block of hydrophobic polymerized monomers. (B(-L³-R³))_{z2} is the second block of catechol polymerized monomers. (C(-L⁴-R⁴))_{z3} is the third block of hydrophilic polymerized monomers. A is a first block monomer backbone moiety. B is a second block monomer backbone moiety. C is a third block monomer backbone moiety. The symbols z1, z2 and z3 are independently integers from 1 to 100. The symbol z5 is 0 or 1. L¹ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In embodiments, R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. L²-R² is a hydrophobic moiety, wherein L² is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In embodiments, R² is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. L³-R³ is a catechol moiety, wherein L³ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R³ is substituted or unsubstituted 1,2-dihydroxyphenyl. L⁴-R⁴ is a hydrophilic moiety, wherein L⁴ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁴ is hydrogen, —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In embodiments, R⁴ is —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. L⁵ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁵ is hydrogen, —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In embodiments, R⁵ is —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In embodiments, z5 is 1. In embodiments, z5 is 0. In embodiments, R¹-L¹ is hydrophobic and may be referred to as an additional hydrophobic moiety. In embodiments, R¹-L¹ is hydrogen. In embodiments, L⁵-R⁵ is hydrophilic and may be referred to herein as an additional hydrophilic moiety. In embodiments, L⁵-R⁵ is hydrogen.

In embodiments, R¹ is independently hydrogen, oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHl₂, —OCHF₂, R⁷-substituted or unsubstituted alkyl (e.g., C₁-C₈ alkyl, C₁-C₆ alkyl, or C₁-C₄ alkyl), R⁷-substituted or unsubstituted heteroalkyl (e.g., 2 to 8 membered heteroalkyl, 2 to 6 membered heteroalkyl, or 2 to 4 membered heteroalkyl), R⁷-substituted or unsubstituted cycloalkyl (e.g., C₃-C₈ cycloalkyl, C₃-C₆ cycloalkyl, or C₅-C₆ cycloalkyl), R⁷-substituted or unsubstituted heterocycloalkyl (e.g., 3 to 8 membered heterocycloalkyl, 3 to 6 membered heterocycloalkyl, or 5 to 6 membered heterocycloalkyl), R⁷-substituted or unsubstituted aryl (e.g., C₆-C₁₀ aryl, C₁₀ aryl, or phenyl), or R⁷-substituted or unsubstituted heteroaryl (e.g., 5 to 10 membered heteroaryl, 5 to 9 membered heteroaryl, or 5 to 6 membered heteroaryl).

In embodiments, R¹ is R⁷-substituted or unsubstituted alkyl (e.g., C₁-C₈ alkyl, C₁-C₆ alkyl, or C₁-C₄ alkyl). In embodiments, R¹ is R⁷-substituted alkyl (e.g., C₁-C₈ alkyl, C₁-C₆ alkyl, or C₁-C₄ alkyl). In embodiments, R¹ is an unsubstituted alkyl (e.g., C₁-C₈ alkyl, C₁-C₆ alkyl, or C₁-C₄ alkyl). In embodiments, R¹ is R⁷-substituted or unsubstituted alkenyl (e.g., C₂-C₁₀). In embodiments, R¹ is an unsubstituted alkenyl. In embodiments, R¹ is a R⁷-substituted alkenyl. In embodiments, R¹ is an unsubstituted C₂-C₁₀ alkenyl. In embodiments, R¹ is an unsubstituted C₂-C₈ alkenyl. In embodiments, R¹ is an unsubstituted ethylenyl.

In embodiments, R¹ is R⁷-substituted or unsubstituted heteroalkyl (e.g., 2 to 8 membered heteroalkyl, 2 to 6 membered heteroalkyl, or 2 to 4 membered heteroalkyl). In embodiments, R¹ is R⁷-substituted heteroalkyl (e.g., 2 to 8 membered heteroalkyl, 2 to 6 membered heteroalkyl, or 2 to 4 membered heteroalkyl). In embodiments, R¹ is an unsubstituted heteroalkyl (e.g., 2 to 8 membered heteroalkyl, 2 to 6 membered heteroalkyl, or 2 to 4 membered heteroalkyl).

In embodiments, R¹ is R⁷-substituted or unsubstituted cycloalkyl (e.g., C₃-C₈ cycloalkyl, C₃-C₆ cycloalkyl, or C₅-C₆ cycloalkyl). In embodiments, R¹ is R⁷-substituted cycloalkyl (e.g., C₃-C₈ cycloalkyl, C₃-C₆ cycloalkyl, or C₅-C₆ cycloalkyl). In embodiments, R¹ is an unsubstituted cycloalkyl (e.g., C₃-C₈ cycloalkyl, C₃-C₆ cycloalkyl, or C₅-C₆ cycloalkyl).

In embodiments, R¹ is R⁷-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹ is R⁷-substituted heterocycloalkyl (e.g., 3 to 8 membered heterocycloalkyl, 3 to 6 membered heterocycloalkyl, or 5 to 6 membered heterocycloalkyl). In embodiments, R¹ is an unsubstituted heterocycloalkyl (e.g., 3 to 8 membered heterocycloalkyl, 3 to 6 membered heterocycloalkyl, or 5 to 6 membered heterocycloalkyl).

In embodiments, R¹ is R⁷-substituted or unsubstituted aryl (e.g., C₆-C₁₀ aryl, C₁₀ aryl, or phenyl). In embodiments, R¹ is R⁷-substituted aryl (e.g., C₆-C₁₀ aryl, C₁₀ aryl, or phenyl). In embodiments, R¹ is an unsubstituted aryl (e.g., C₆-C₁₀ aryl, or phenyl). In embodiments, R¹ is an unsubstituted phenyl.

In embodiments, R¹ is R⁷-substituted or unsubstituted heteroaryl (e.g., 5 to 10 membered heteroaryl, 5 to 9 membered heteroaryl, or 5 to 6 membered heteroaryl). In embodiments, R¹ is R⁷-substituted heteroaryl (e.g., 5 to 10 membered heteroaryl, 5 to 9 membered heteroaryl, or 5 to 6 membered heteroaryl). In embodiments, R¹ is an unsubstituted heteroaryl (e.g., 5 to 10 membered heteroaryl, 5 to 9 membered heteroaryl, or 5 to 6 membered heteroaryl).

In embodiments, R¹ is a hydrophobic drug moiety, a targeting moiety or a detectable moiety. In embodiments, R¹ is a hydrophobic drug moiety. In embodiments, R¹ is a drug moiety. In embodiments, R¹ is a targeting moiety. In embodiments, R¹ is a detectable moiety. In embodiments, R¹ is hydrogen. In embodiments, R¹ is not a drug moiety, targeting moiety or detectable moiety.

R⁷ is independently oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHl₂, —OCHF₂, R⁸-substituted or unsubstituted alkyl (e.g., C₁-C₈ alkyl, C₁-C₆ alkyl, or C₁-C₄ alkyl), R⁸-substituted or unsubstituted heteroalkyl (e.g., 2 to 8 membered heteroalkyl, 2 to 6 membered heteroalkyl, or 2 to 4 membered heteroalkyl), R⁸-substituted or unsubstituted cycloalkyl (e.g., C₃-C₈ cycloalkyl, C₃-C₆ cycloalkyl, or C₅-C₆ cycloalkyl), R⁸-substituted or unsubstituted heterocycloalkyl (e.g., 3 to 8 membered heterocycloalkyl, 3 to 6 membered heterocycloalkyl, or 5 to 6 membered heterocycloalkyl), R⁸-substituted or unsubstituted aryl (e.g., C₆-C₁₀ aryl, C₁₀ aryl, or phenyl), or R⁸-substituted or unsubstituted heteroaryl (e.g., 5 to 10 membered heteroaryl, 5 to 9 membered heteroaryl, or 5 to 6 membered heteroaryl).

In embodiments, L¹ is a bond, —O—, —S—, —NH—, —C(O)—, —C(O)O—, —C(O)NH—, R⁹-substituted or unsubstituted alkylene (e.g., C₁-C₈ alkylene, C₁-C₆ alkylene, or C₁-C₄ alkylene), R⁹-substituted or unsubstituted heteroalkylene (e.g., 2 to 8 membered heteroalkylene, 2 to 6 membered heteroalkylene, or 2 to 4 membered heteroalkylene), R⁹-substituted or unsubstituted cycloalkylene (e.g., C₃-C₈ cycloalkylene, C₃-C₆ cycloalkylene, or C₅-C₆ cycloalkylene), R⁹-substituted or unsubstituted heterocycloalkylene (e.g., 3 to 8 membered heterocycloalkylene, 3 to 6 membered heterocycloalkylene, or 5 to 6 membered heterocycloalkylene), R⁹-substituted or unsubstituted arylene (e.g., C₆-C₁₀ arylene, C₁₀ arylene, or phenylene), or R⁹-substituted or unsubstituted heteroarylene (e.g., 5 to 10 membered heteroarylene, 5 to 9 membered heteroarylene, or 5 to 6 membered heteroarylene). In embodiments, L¹ is a bond, —O—, —S—, —NH—, —C(O)—, —C(O)O—, —C(O)NH—, R⁹-substituted or unsubstituted alkenylene (e.g., C₂-C₈ alkenylene, C₂-C₆ alkenylene, or C₂-C₄ alkenylene). In embodiments, L¹ is a substituted or unsubstituted ethylenyl.

In embodiments, L¹ is R⁹-substituted or unsubstituted alkylene (e.g., C₁-C₈ alkylene, C₁-C₆ alkylene, or C₁-C₄ alkylene). In embodiments, L¹ is R⁹-substituted alkylene (e.g., C₁-C₈ alkylene, C₁-C₆ alkylene, or C₁-C₄ alkylene). In embodiments, L¹ is an unsubstituted alkylene (e.g., C₁-C₈ alkylene, C₁-C₆ alkylene, or C₁-C₄ alkylene).

In embodiments, L¹ is R⁹-substituted or unsubstituted heteroalkylene (e.g., 2 to 8 membered heteroalkylene, 2 to 6 membered heteroalkylene, or 2 to 4 membered heteroalkylene). In embodiments, L¹ is R⁹-substituted heteroalkylene (e.g., 2 to 8 membered heteroalkylene, 2 to 6 membered heteroalkylene, or 2 to 4 membered heteroalkylene). In embodiments, L¹ is an unsubstituted heteroalkylene (e.g., 2 to 8 membered heteroalkylene, 2 to 6 membered heteroalkylene, or 2 to 4 membered heteroalkylene).

In embodiments, L¹ is R⁹-substituted or unsubstituted cycloalkylene (e.g., C₃-C₈ cycloalkylene, C₃-C₆ cycloalkylene, or C₅-C₆ cycloalkylene). In embodiments, L¹ is R⁹-substituted cycloalkylene (e.g., C₃-C₈ cycloalkylene, C₃-C₆ cycloalkylene, or C₅-C₆ cycloalkylene). In embodiments, L¹ is an unsubstituted cycloalkylene (e.g., C₃-C₈ cycloalkylene, C₃-C₆ cycloalkylene, or C₅-C₆ cycloalkylene).

In embodiments, L¹ is R⁹-substituted or unsubstituted heterocycloalkylene (e.g., 3 to 8 membered heterocycloalkylene, 3 to 6 membered heterocycloalkylene, or 5 to 6 membered heterocycloalkylene). In embodiments, L¹ is R⁹-substituted heterocycloalkylene (e.g., 3 to 8 membered heterocycloalkylene, 3 to 6 membered heterocycloalkylene, or 5 to 6 membered heterocycloalkylene). In embodiments, L¹ is an unsubstituted heterocycloalkylene (e.g., 3 to 8 membered heterocycloalkylene, 3 to 6 membered heterocycloalkylene, or 5 to 6 membered heterocycloalkylene).

In embodiments, L¹ is R⁹-substituted or unsubstituted arylene (e.g., C₆-C₁₀ arylene, C₁₀ arylene, or phenylene). In embodiments, L¹ is R⁹-substituted arylene (e.g., C₆-C₁₀ arylene, C₁₀ arylene, or phenylene). In embodiments, L¹ is an unsubstituted arylene (e.g., C₆-C₁₀ arylene, C₁₀ arylene, or phenylene).

In embodiments, L¹ is R⁹-substituted or unsubstituted heteroarylene (e.g., 5 to 10 membered heteroarylene, 5 to 9 membered heteroarylene, or 5 to 6 membered heteroarylene). In embodiments, L¹ is R⁹-substituted heteroarylene (e.g., 5 to 10 membered heteroarylene, 5 to 9 membered heteroarylene, or 5 to 6 membered heteroarylene). In embodiments, L¹ is an unsubstituted heteroarylene (e.g., 5 to 10 membered heteroarylene, 5 to 9 membered heteroarylene, or 5 to 6 membered heteroarylene). In embodiments, L¹ is a bond.

R⁹ is independently oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHl₂, —OCHF₂, R¹⁰-substituted or unsubstituted alkyl (e.g., C₁-C₈ alkyl, C₁-C₆ alkyl, or C₁-C₄ alkyl), R¹⁰-substituted or unsubstituted heteroalkyl (e.g., 2 to 8 membered heteroalkyl, 2 to 6 membered heteroalkyl, or 2 to 4 membered heteroalkyl), R¹⁰-substituted or unsubstituted cycloalkyl (e.g., C₃-C₈ cycloalkyl, C₃-C₆ cycloalkyl, or C₅-C₆ cycloalkyl), R¹⁰-substituted or unsubstituted heterocycloalkyl (e.g., 3 to 8 membered heterocycloalkyl, 3 to 6 membered heterocycloalkyl, or 5 to 6 membered heterocycloalkyl), R¹⁰-substituted or unsubstituted aryl (e.g., C₆-C₁₀ aryl, C₁₀ aryl, or phenyl), or R¹⁰-substituted or unsubstituted heteroaryl (e.g., 5 to 10 membered heteroaryl, 5 to 9 membered heteroaryl, or 5 to 6 membered heteroaryl).

In embodiments, A is a first block monomer backbone moiety. Thus in embodiments, (A(-L²-R²))_{z1} has the formula (-L^{1A}-A¹(-L²-R²)-L^{2A}-)_{z1}. L^{1A} and L^{2A} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. A¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, A¹ is R²⁵-substituted or unsubstituted alkylene (e.g., C₁-C₈ alkylene, C₁-C₆ alkylene, or C₁-C₄ alkylene), R²⁵-substituted or unsubstituted heteroalkylene (e.g., 2 to 8 membered heteroalkylene, 2 to 6 membered heteroalkylene, or 2 to 4 membered heteroalkylene), R²⁵-substituted or unsubstituted cycloalkylene (e.g., C₃-C₈ cycloalkylene, C₃-C₆ cycloalkylene, or C₅-C₆ cycloalkylene), R²⁵-substituted or unsubstituted heterocycloalkylene (e.g., 3 to 8 membered heterocycloalkylene, 3 to 6 membered heterocycloalkylene, or 5 to 6 membered heterocycloalkylene), R²⁵-substituted or unsubstituted arylene (e.g., C₆-C₁₀ arylene, or phenylene), or R²⁵-substituted or unsubstituted heteroarylene (e.g., 5 to 10 membered heteroarylene, 5 to 9 membered heteroarylene, or 5 to 6 membered heteroarylene).

In embodiments, A¹ is R²⁵-substituted or unsubstituted alkylene (e.g., C₁-C₈ alkylene, C₁-C₆ alkylene, or C₁-C₄ alkylene). In embodiments, A¹ is R²⁵-substituted alkylene (e.g., C₁-C₈ alkylene, C₁-C₆ alkylene, or C₁-C₄ alkylene). In embodiments, A¹ is an unsubstituted alkylene (e.g., C₁-C₈ alkylene, C₁-C₆ alkylene, or C₁-C₄ alkylene).

In embodiments, A¹ is R²⁵-substituted or unsubstituted heteroalkylene (e.g., 2 to 8 membered heteroalkylene, 2 to 6 membered heteroalkylene, or 2 to 4 membered heteroalkylene). In embodiments, A¹ is R²⁵-substituted heteroalkylene (e.g., 2 to 8 membered heteroalkylene, 2 to 6 membered heteroalkylene, or 2 to 4 membered heteroalkylene). In embodiments, A¹ is an unsubstituted heteroalkylene (e.g., 2 to 8 membered heteroalkylene, 2 to 6 membered heteroalkylene, or 2 to 4 membered heteroalkylene).

In embodiments, A¹ is R²⁵-substituted or unsubstituted cycloalkylene (e.g., C₃-C₈ cycloalkylene, C₃-C₆ cycloalkylene, or C₅-C₆ cycloalkylene). In embodiments, A¹ is R²⁵-substituted cycloalkylene (e.g., C₃-C₈ cycloalkylene, C₃-C₆ cycloalkylene, or C₅-C₆ cycloalkylene). In embodiments, A¹ is an unsubstituted cycloalkylene (e.g., C₃-C₈ cycloalkylene, C₃-C₆ cycloalkylene, or C₅-C₆ cycloalkylene).

In embodiments, A¹ is R²⁵-substituted or unsubstituted heterocycloalkylene (e.g., 3 to 8 membered heterocycloalkylene, 3 to 6 membered heterocycloalkylene, or 5 to 6 membered heterocycloalkylene). In embodiments, A¹ is R²⁵-substituted heterocycloalkylene (e.g., 3 to 8 membered heterocycloalkylene, 3 to 6 membered heterocycloalkylene, or 5 to 6 membered heterocycloalkylene). In embodiments, A¹ is an unsubstituted heterocycloalkylene (e.g., 3 to 8 membered heterocycloalkylene, 3 to 6 membered heterocycloalkylene, or 5 to 6 membered heterocycloalkylene).

In embodiments, A¹ is R²⁵-substituted or unsubstituted arylene (e.g., C₆-C₁₀ arylene, C₁₀ arylene, or phenylene). In embodiments, A¹ is R²⁵-substituted arylene (e.g., C₆-C₁₀ arylene, C₁₀ arylene, or phenylene). In embodiments, A¹ is an unsubstituted arylene (e.g., C₆-C₁₀ arylene, C₁₀ arylene, or phenylene).

In embodiments, A¹ is R²⁵-substituted or unsubstituted heteroarylene (e.g., 5 to 10 membered heteroarylene, 5 to 9 membered heteroarylene, or 5 to 6 membered heteroarylene). In embodiments, A¹ is R²⁵-substituted heteroarylene (e.g., 5 to 10 membered heteroarylene, 5 to 9 membered heteroarylene, or 5 to 6 membered heteroarylene). In embodiments, A¹ is an unsubstituted heteroarylene (e.g., 5 to 10 membered heteroarylene, 5 to 9 membered heteroarylene, or 5 to 6 membered heteroarylene).

R²⁵ is independently oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHl₂, —OCHF₂, R²⁶-substituted or unsubstituted alkyl (e.g., C₁-C₈ alkyl, C₁-C₆ alkyl, or C₁-C₄ alkyl), R²⁶-substituted or unsubstituted heteroalkyl (e.g., 2 to 8 membered heteroalkyl, 2 to 6 membered heteroalkyl, or 2 to 4 membered heteroalkyl), R²⁶-substituted or unsubstituted cycloalkyl (e.g., C₃-C₈ cycloalkyl, C₃-C₆ cycloalkyl, or C₅-C₆ cycloalkyl), R²⁶-substituted or unsubstituted heterocycloalkyl (e.g., 3 to 8 membered heterocycloalkyl, 3 to 6 membered heterocycloalkyl, or 5 to 6 membered heterocycloalkyl), R²⁶-substituted or unsubstituted aryl (e.g., C₆-C₁₀ aryl, C₁₀ aryl, or phenyl), or R²⁶-substituted or unsubstituted heteroaryl (e.g., 5 to 10 membered heteroaryl, 5 to 9 membered heteroaryl, or 5 to 6 membered heteroaryl).

In embodiments, R²⁵ is oxo, —OH, —NH₂, —COOH, or —CONH₂. In embodiments, R²⁵ is oxo.

In embodiments, L^{1A} is a bond, —O—, —S—, —NH—, —C(O)—, —C(O)O—, —C(O)NH—, R^{22A}-substituted or unsubstituted alkylene (e.g., C₁-C₈ alkylene, C₁-C₆ alkylene, or C₁-C₄ alkylene), R^{22A}-substituted or unsubstituted heteroalkylene (e.g., 2 to 8 membered heteroalkylene, 2 to 6 membered heteroalkylene, or 2 to 4 membered heteroalkylene), R^{22A}-substituted or unsubstituted cycloalkylene (e.g., C₃-C₈ cycloalkylene, C₃-C₆ cycloalkylene, or C₅-C₆ cycloalkylene), R^{22A}-substituted or unsubstituted heterocycloalkylene (e.g., 3 to 8 membered heterocycloalkylene, 3 to 6 membered heterocycloalkylene, or 5 to 6 membered heterocycloalkylene), R^{22A}-substituted or unsubstituted arylene (e.g., C₆-C₁₀ arylene, C₁₀ arylene, or phenylene), or R^{22A}-substituted or unsubstituted heteroarylene (e.g., 5 to 10 membered heteroarylene, 5 to 9 membered heteroarylene, or 5 to 6 membered heteroarylene). In embodiments, L^{1A} is a bond, —O—, —S—, —NH—, —C(O)—, —C(O)O—, —C(O)NH—, R^{22A}-substituted or unsubstituted alkenylene (e.g., C₂-C₈ alkenylene, C₂-C₆ alkenylene, or C₂-C₄ alkenylene). In embodiments, L^{1A} is a substituted or unsubstituted ethylenyl.

In embodiments, L^{2A} is a bond, —O—, —S—, —NH—, —C(O)—, —C(O)O—, —C(O)NH—, R^{23A}-substituted or unsubstituted alkylene (e.g., C₁-C₈ alkylene, C₁-C₆ alkylene, or C₁-C₄ alkylene), R^{23A}-substituted or unsubstituted heteroalkylene (e.g., 2 to 8 membered heteroalkylene, 2 to 6 membered heteroalkylene, or 2 to 4 membered heteroalkylene), R^{23A}-substituted or unsubstituted cycloalkylene (e.g., C₃-C₈ cycloalkylene, C₃-C₆ cycloalkylene, or C₅-C₆ cycloalkylene), R^{23A}-substituted or unsubstituted heterocycloalkylene (e.g., 3 to 8 membered heterocycloalkylene, 3 to 6 membered heterocycloalkylene, or 5 to 6 membered heterocycloalkylene), R^{23A}-substituted or unsubstituted arylene (e.g., C₆-C₁₀ arylene, C₁₀ arylene, or phenylene), or R^{23A}-substituted or unsubstituted heteroarylene (e.g., 5 to 10 membered heteroarylene, 5 to 9 membered heteroarylene, or 5 to 6 membered heteroarylene). In embodiments, L^{2A} is a bond, —O—, —S—, —NH—, —C(O)—, —C(O)O—, —C(O)NH—, R^{23A}-substituted or unsubstituted alkenylene (e.g., C₂-C₈ alkenylene, C₂-C₆ alkenylene, or C₂-C₄ alkenylene). In embodiments, L^{2A} is a substituted or unsubstituted ethylenyl.

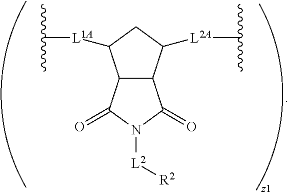

In embodiments, (A(-L²-R²))_{z1} has the formula:

In embodiments, L^{1A} and L^{2A} are independently a bond, —O—, —S—, —NH—, —C(O)—, —C(O)O—, —C(O)NH—, substituted or unsubstituted alkylene, substituted or unsubstituted heteroalkylene.

In embodiments, L²-R² is a hydrophobic moiety. In embodiments, L² is a hydrophobic moiety. In embodiments, R² is a hydrophobic moiety. In embodiments, L²-R² is substituted or unsubstituted alkyl. In embodiments, L²-R² is a substituted alkyl. In embodiments, L²-R² is an unsubstituted alkyl. In embodiments, L²-R² is a substituted C₁-C₁₀ alkyl. In embodiments, L²-R² is a substituted C₁-C₁₀ alkyl. In embodiments, L²-R² is a substituted C₄-C₁₀ alkyl. In embodiments, L²-R² is a substituted C₆-C₁₀ alkyl. In embodiments, L²-R² is a substituted C₆-C₈ alkyl. In embodiments, L²-R² is an unsubstituted C₁-C₁₀ alkyl. In embodiments, L²-R² is an unsubstituted C₄-C₁₀ alkyl. In embodiments, L²-R² is an unsubstituted C₆-C₁₀ alkyl. In embodiments, L²-R² is an unsubstituted C₆ alkyl. In embodiments, L²-R² is an unsubstituted C₇ alkyl. In embodiments, L²-R² is an unsubstituted C₈ alkyl. In embodiments, L²-R² is an unsubstituted C₉ alkyl. In embodiments, L²-R² is an unsubstituted C₁₀ alkyl. In embodiments, L²-R² is an unsubstituted C₆-C₈ alkyl. In embodiments, L²-R² is a hydrophobic moiety. In embodiments, L² is a hydrophobic moiety. In embodiments, R² is a hydrophobic moiety. In embodiments, L²-R² is R¹¹-substituted or unsubstituted alkyl. In embodiments, L²-R² is a R¹¹-substituted alkyl. In embodiments, L²-R² is a R¹¹-substituted C₁-C₁₀ alkyl. In embodiments, L²-R² is a R¹¹-substituted C₁-C₁₀ alkyl. In embodiments, L²-R² is a R¹¹-substituted C₄-C₁₀ alkyl. In embodiments, L²-R² is a R¹¹-substituted C₆-C₁₀ alkyl. In embodiments, L²-R² is a R¹¹-substituted C₆-C₈ alkyl. In embodiments, L²-R² is an R¹¹-substituted C₆ alkyl. In embodiments, L²-R² is an R¹¹-substituted C₇ alkyl. In embodiments, L²-R² is an R¹¹-substituted C₈ alkyl. In embodiments, L²-R² is an R¹¹-substituted C₉ alkyl. In embodiments, L²-R² is an R¹¹-substituted C₁₀ alkyl.

In embodiments, L²-R² is

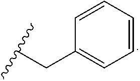 [SMILES: *Cc1ccccc1]

In embodiments, L² is a substituted or unsubstituted C₁-C₈ alkylene and R² is phenyl. In embodiments, L² a substituted or unsubstituted C₁-C₆ alkylene and R² is phenyl. In embodiments, L² a substituted or unsubstituted C₁-C₄ alkylene and R² is phenyl. In embodiments, L² a substituted or unsubstituted C₁-C₂ alkylene and R² is phenyl. In embodiments, L² a substituted or unsubstituted methylene and R² is phenyl. In embodiments, L² an unsubstituted methylene and R² is phenyl. In embodiments, R² is unsubstituted phenyl and L² is unsubstituted C₁-C₁₀ alkylene (e.g. unbranched C₁-C₁₀ alkyl). In embodiments, R² is unsubstituted phenyl and L² is unsubstituted C₁-C₅ alkylene (e.g. unbranched C₁-C₅ alkyl).

In embodiments, L² is a bond, —O—, —S—, —NH—, —C(O)—, —C(O)O—, —C(O)NH—, R¹¹-substituted or unsubstituted alkylene (e.g., C₁-C₈ alkylene, C₁-C₆ alkylene, or C₁-C₄ alkylene), R¹¹-substituted or unsubstituted heteroalkylene (e.g., 2 to 8 membered heteroalkylene, 2 to 6 membered heteroalkylene, or 2 to 4 membered heteroalkylene), R¹¹-substituted or unsubstituted cycloalkylene (e.g., C₃-C₈ cycloalkylene, C₃-C₆ cycloalkylene, or C₅-C₆ cycloalkylene), R¹¹-substituted or unsubstituted heterocycloalkylene (e.g., 3 to 8 membered heterocycloalkylene, 3 to 6 membered heterocycloalkylene, or 5 to 6 membered heterocycloalkylene), R¹¹-substituted or unsubstituted arylene (e.g., C₆-C₁₀ arylene, C₁₀ arylene, or phenylene), or R¹¹-substituted or unsubstituted heteroarylene (e.g., 5 to 10 membered heteroarylene, 5 to 9 membered heteroarylene, or 5 to 6 membered heteroarylene). In embodiments, L² is a bond.

In embodiments, R² is hydrogen, R¹²-substituted or unsubstituted alkyl (e.g., C₁-C₈ alkyl, C₁-C₆ alkyl, or C₁-C₄ alkyl), R¹²-substituted or unsubstituted heteroalkyl (e.g., 2 to 8 membered heteroalkyl, 2 to 6 membered heteroalkyl, or 2 to 4 membered heteroalkyl), R¹²-substituted or unsubstituted cycloalkyl (e.g., C₃-C₈ cycloalkyl, C₃-C₆ cycloalkyl, or C₅-C₆ cycloalkyl), R¹²-substituted or unsubstituted heterocycloalkyl (e.g., 3 to 8 membered heterocycloalkyl, 3 to 6 membered heterocycloalkyl, or 5 to 6 membered heterocycloalkyl), R¹²-substituted or unsubstituted aryl (e.g., C₆-C₁₀ aryl, C₁₀ aryl, or phenyl), or R¹²-substituted or unsubstituted heteroaryl (e.g., 5 to 10 membered heteroaryl, 5 to 9 membered heteroaryl, or 5 to 6 membered heteroaryl). In embodiments, R² is hydrogen.

In embodiments, B is a second block monomer backbone moiety. Thus, in embodiments, (B(-L³-R³))_{z2} has the formula (-L^{1B}-B¹(-L³-R³)-L_{2B}-)_{z2}. L^{1B} and L^{2B} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. B¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, B¹ is R²⁷-substituted or unsubstituted alkylene (e.g., C₁-C₈ alkylene, C₁-C₆ alkylene, or C₁-C₄ alkylene), R²⁷-substituted or unsubstituted heteroalkylene (e.g., 2 to 8 membered heteroalkylene, 2 to 6 membered heteroalkylene, or 2 to 4 membered heteroalkylene), R²⁷-substituted or unsubstituted cycloalkylene (e.g., C₃-C₈ cycloalkylene, C₃-C₆ cycloalkylene, or C₅-C₆ cycloalkylene), R²⁷-substituted or unsubstituted heterocycloalkylene (e.g., 3 to 8 membered heterocycloalkylene, 3 to 6 membered heterocycloalkylene, or 5 to 6 membered heterocycloalkylene), R²⁷-substituted or unsubstituted arylene (e.g., C₆-C₁₀ arylene, C₁₀ arylene, or phenylene), or R²⁷-substituted or unsubstituted heteroarylene (e.g., 5 to 10 membered heteroarylene, 5 to 9 membered heteroarylene, or 5 to 6 membered heteroarylene).

In embodiments, B¹ is R²⁷-substituted or unsubstituted alkylene (e.g., C₁-C₈ alkylene, C₁-C₆ alkylene, or C₁-C₄ alkylene). In embodiments, B¹ is R²⁷-substituted alkylene (e.g., C₁-C₈ alkylene, C₁-C₆ alkylene, or C₁-C₄ alkylene). In embodiments, B¹ is an unsubstituted alkylene (e.g., C₁-C₈ alkylene, C₁-C₆ alkylene, or C₁-C₄ alkylene).

In embodiments, B¹ is R²⁷-substituted or unsubstituted heteroalkylene (e.g., 2 to 8 membered heteroalkylene, 2 to 6 membered heteroalkylene, or 2 to 4 membered heteroalkylene). In embodiments, B¹ is R²⁷-substituted heteroalkylene (e.g., 2 to 8 membered heteroalkylene, 2 to 6 membered heteroalkylene, or 2 to 4 membered heteroalkylene). In embodiments, B¹ is an unsubstituted heteroalkylene (e.g., 2 to 8 membered heteroalkylene, 2 to 6 membered heteroalkylene, or 2 to 4 membered heteroalkylene).

In embodiments, B¹ is R²⁷-substituted or unsubstituted cycloalkylene (e.g., C₃-C₈ cycloalkylene, C₃-C₆ cycloalkylene, or C₅-C₆ cycloalkylene). In embodiments, B¹ is R²⁷-substituted cycloalkylene (e.g., C₃-C₈ cycloalkylene, C₃-C₆ cycloalkylene, or C₅-C₆ cycloalkylene). In embodiments, B¹ is an unsubstituted cycloalkylene (e.g., C₃-C₈ cycloalkylene, C₃-C₆ cycloalkylene, or C₅-C₆ cycloalkylene).

In embodiments, B¹ is R²⁷-substituted or unsubstituted heterocycloalkylene (e.g., 3 to 8 membered heterocycloalkylene, 3 to 6 membered heterocycloalkylene, or 5 to 6 membered heterocycloalkylene). In embodiments, B¹ is R²⁷-substituted heterocycloalkylene (e.g., 3 to 8 membered heterocycloalkylene, 3 to 6 membered heterocycloalkylene, or 5 to 6 membered heterocycloalkylene). In embodiments, B¹ is an unsubstituted heterocycloalkylene (e.g., 3 to 8 membered heterocycloalkylene, 3 to 6 membered heterocycloalkylene, or 5 to 6 membered heterocycloalkylene).

In embodiments, B¹ is R²⁷-substituted or unsubstituted arylene (e.g., C₆-C₁₀ arylene, C₁₀ arylene, or phenylene). In embodiments, B¹ is R²⁷-substituted arylene (e.g., C₆-C₁₀ arylene, C₁₀ arylene, or phenylene). In embodiments, B¹ is an unsubstituted arylene (e.g., C₆-C₁₀ arylene, C₁₀ arylene, or phenylene).

In embodiments, B¹ is R²⁷-substituted or unsubstituted heteroarylene (e.g., 5 to 10 membered heteroarylene, 5 to 9 membered heteroarylene, or 5 to 6 membered heteroarylene). In embodiments, B¹ is R²⁷-substituted heteroarylene (e.g., 5 to 10 membered heteroarylene, 5 to 9 membered heteroarylene, or 5 to 6 membered heteroarylene). In embodiments, B¹ is an unsubstituted heteroarylene (e.g., 5 to 10 membered heteroarylene, 5 to 9 membered heteroarylene, or 5 to 6 membered heteroarylene).

R²⁷ is independently oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHl₂, —OCHF₂, R²⁸-substituted or unsubstituted alkyl (e.g., C₁-C₈ alkyl, C₁-C₆ alkyl, or C₁-C₄ alkyl), R²⁸-substituted or unsubstituted heteroalkyl (e.g., 2 to 8 membered heteroalkyl, 2 to 6 membered heteroalkyl, or 2 to 4 membered heteroalkyl), R²⁸-substituted or unsubstituted cycloalkyl (e.g., C₃-C₈ cycloalkyl, C₃-C₆ cycloalkyl, or C₅-C₆ cycloalkyl), R²⁸-substituted or unsubstituted heterocycloalkyl (e.g., 3 to 8 membered heterocycloalkyl, 3 to 6 membered heterocycloalkyl, or 5 to 6 membered heterocycloalkyl), R²⁸-substituted or unsubstituted aryl (e.g., C₆-C₁₀ aryl, C₁₀ aryl, or phenyl), or R²⁸-substituted or unsubstituted heteroaryl (e.g., 5 to 10 membered heteroaryl, 5 to 9 membered heteroaryl, or 5 to 6 membered heteroaryl).

In embodiments, R²⁷ is oxo, —OH, —NH₂, —COOH, or —CONH₂. In embodiments, R²⁷ is oxo.

In embodiments, L^{1B} is a bond, —O—, —S—, —NH—, —C(O)—, —C(O)O—, —C(O)NH—, R^{22B}-substituted or unsubstituted alkylene (e.g., C₁-C₈ alkylene, C₁-C₆ alkylene, or C₁-C₄ alkylene), R^{22B}-substituted or unsubstituted heteroalkylene (e.g., 2 to 8 membered heteroalkylene, 2 to 6 membered heteroalkylene, or 2 to 4 membered heteroalkylene), R^{22B}-substituted or unsubstituted cycloalkylene (e.g., C₃-C₈ cycloalkylene, C₃-C₆ cycloalkylene, or C₅-C₆ cycloalkylene), R^{22B}-substituted or unsubstituted heterocycloalkylene (e.g., 3 to 8 membered heterocycloalkylene, 3 to 6 membered heterocycloalkylene, or 5 to 6 membered heterocycloalkylene), R^{22B}-substituted or unsubstituted arylene (e.g., C₆-C₁₀ arylene, C₁₀ arylene, or phenylene), or R^{22B}-substituted or unsubstituted heteroarylene (e.g., 5 to 10 membered heteroarylene, 5 to 9 membered heteroarylene, or 5 to 6 membered heteroarylene). In embodiments, L^{1B} is a bond, —O—, —S—, —NH—, —C(O)—, —C(O)O—, —C(O)NH—, R^{22B}-substituted or unsubstituted alkenylene (e.g., C₂-C₈ alkenylene, C₂-C₆ alkenylene, or C₂-C₄ alkenylene). In embodiments, L^{1B} is a substituted or unsubstituted ethylenyl.

In embodiments, L^{2B} is a bond, —O—, —S—, —NH—, —C(O)—, —C(O)O—, —C(O)NH—, R^{23B}-substituted or unsubstituted alkylene (e.g., C₁-C₈ alkylene, C₁-C₆ alkylene, or C₁-C₄ alkylene), R^{23B}-substituted or unsubstituted heteroalkylene (e.g., 2 to 8 membered heteroalkylene, 2 to 6 membered heteroalkylene, or 2 to 4 membered heteroalkylene), R^{23B}-substituted or unsubstituted cycloalkylene (e.g., C₃-C₈ cycloalkylene, C₃-C₆ cycloalkylene, or C₅-C₆ cycloalkylene), R^{23B}-substituted or unsubstituted heterocycloalkylene (e.g., 3 to 8 membered heterocycloalkylene, 3 to 6 membered heterocycloalkylene, or 5 to 6 membered heterocycloalkylene), R^{23B}-substituted or unsubstituted arylene (e.g., C₆-C₁₀ arylene, C₁₀ arylene, or phenylene), or R^{23B}-substituted or unsubstituted heteroarylene (e.g., 5 to 10 membered heteroarylene, 5 to 9 membered heteroarylene, or 5 to 6 membered heteroarylene). In embodiments, L^{2B} is a bond, —O—, —S—, —NH—, —C(O)—, —C(O)O—, —C(O)NH—, R^{23B}-substituted or unsubstituted alkenylene (e.g., C₂-C₈ alkenylene, C₂-C₆ alkenylene, or C₂-C₄ alkenylene). In embodiments, L^{2B} is a substituted or unsubstituted ethylenyl.

In embodiments, (B(-L³-R³))_{z2} has the formula:

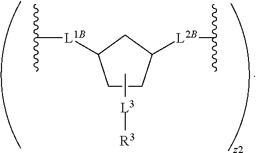

In embodiments, L^{1B} and L^{2B} are independently a bond, —O—, —S—, —NH—, —C(O)—, —C(O)O—, —C(O)NH—, substituted or unsubstituted alkylene, substituted or unsubstituted heteroalkylene.

In embodiments, L³-R³ is a catechol moiety. In embodiments, L³-R³

is:

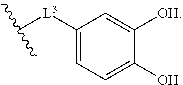

In embodiments, L³ is unsubstituted C₁-C₁₀ alkylene (e.g. unbranched C₁-C₁₀ alkyl). In embodiments, L³ is unsubstituted C₁-C₅ alkylene (e.g. unbranched C₁-C₅ alkyl). In embodiments, L³-R³ is:

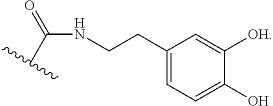 [SMILES: *C(=O)NCCc1ccc(O)c(O)c1]

In embodiments, L³ is a bond, —O—, —S—, —NH—, —C(O)—, —C(O)O—, —C(O)NH—, R¹³-substituted or unsubstituted alkylene (e.g., C₁-C₈ alkylene, C₁-C₆ alkylene, or C₁-C₄ alkylene), R¹³-substituted or unsubstituted heteroalkylene (e.g., 2 to 8 membered heteroalkylene, 2 to 6 membered heteroalkylene, or 2 to 4 membered heteroalkylene), R¹³-substituted or unsubstituted cycloalkylene (e.g., C₃-C₈ cycloalkylene, C₃-C₆ cycloalkylene, or C₅-C₆ cycloalkylene), R¹³-substituted or unsubstituted heterocycloalkylene (e.g., 3 to 8 membered heterocycloalkylene, 3 to 6 membered heterocycloalkylene, or 5 to 6 membered heterocycloalkylene), R¹³-substituted or unsubstituted arylene (e.g., C₆-C₁₀ arylene, C₁₀ arylene, or phenylene), or R¹³-substituted or unsubstituted heteroarylene (e.g., 5 to 10 membered heteroarylene, 5 to 9 membered heteroarylene, or 5 to 6 membered heteroarylene).

In embodiments, L³ is —C(O)—N(H)—(CH₂)_{z4}-. The symbol z4 is an integer from 1 to 20. In embodiments, z4 is an integer from 1 to 15. In embodiments, z4 is an integer from 1 to 10. In embodiments, z4 is an integer from 1 to 4. In embodiments, z4 is 1 or 2. In embodiments, z4 is 1. In embodiments, z4 is 2. In embodiments, z4 is 3. In embodiments, z4 is 4. In embodiments, z4 is 5. In embodiments, z4 is 6. In embodiments, z4 is 7. In embodiments, z4 is 8. In embodiments, z4 is 9. In embodiments, z4 is 10. In embodiments, z4 is 11. In embodiments, z4 is 12. In embodiments, z4 is 13. In embodiments, z4 is 14. In embodiments, z4 is 15. In embodiments, z4 is 16. In embodiments, z4 is 17. In embodiments, z4 is 18. In embodiments, z4 is 19. In embodiments, z4 is 20.

R³ is substituted or unsubstituted 1,2-dihydroxyphenyl. In embodiments, R³ is an unsubstituted 1,2-dihydroxyphenyl. In embodiments, R³ is a substituted 1,2-dihydroxyphenyl. In embodiments, R³ is a R¹⁴-substituted or unsubstituted 1,2-dihydroxyphenyl. In embodiments, R³ is a R¹⁴-substituted 1,2-dihydroxyphenyl.

In embodiments, C is a third block monomer backbone moiety. Thus, in embodiments, (C(-L⁴-R⁴))_{z3} has the formula (-L^{1C}-C¹(-L⁴-R⁴)-L^{2C}-)_{z3}. L^{1C} and L^{2C} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. C¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, C¹ is R²⁹-substituted or unsubstituted alkylene (e.g., C₁-C₈ alkylene, C₁-C₆ alkylene, or C₁-C₄ alkylene), R²⁹-substituted or unsubstituted heteroalkylene (e.g., 2 to 8 membered heteroalkylene, 2 to 6 membered heteroalkylene, or 2 to 4 membered heteroalkylene), R²⁹-substituted or unsubstituted cycloalkylene (e.g., C₃-C₈ cycloalkylene, C₃-C₆ cycloalkylene, or C₅-C₆ cycloalkylene), R²⁹-substituted or unsubstituted heterocycloalkylene (e.g., 3 to 8 membered heterocycloalkylene, 3 to 6 membered heterocycloalkylene, or 5 to 6 membered heterocycloalkylene), R²⁹-substituted or unsubstituted arylene (e.g., C₆-C₁₀ arylene, C₁₀ arylene, or phenylene), or R²⁹-substituted or unsubstituted heteroarylene (e.g., 5 to 10 membered heteroarylene, 5 to 9 membered heteroarylene, or 5 to 6 membered heteroarylene).

In embodiments, C¹ is R²⁹-substituted or unsubstituted alkylene (e.g., C₁-C₈ alkylene, C₁-C₆ alkylene, or C₁-C₄ alkylene). In embodiments, C¹ is R²⁹-substituted alkylene (e.g., C₁-C₈ alkylene, C₁-C₆ alkylene, or C₁-C₄ alkylene). In embodiments, C¹ is an unsubstituted alkylene (e.g., C₁-C₈ alkylene, C₁-C₆ alkylene, or C₁-C₄ alkylene).

In embodiments, C¹ is R²⁹-substituted or unsubstituted heteroalkylene (e.g., 2 to 8 membered heteroalkylene, 2 to 6 membered heteroalkylene, or 2 to 4 membered heteroalkylene). In embodiments, C¹ is R²⁹-substituted heteroalkylene (e.g., 2 to 8 membered heteroalkylene, 2 to 6 membered heteroalkylene, or 2 to 4 membered heteroalkylene). In embodiments, C¹ is an unsubstituted heteroalkylene (e.g., 2 to 8 membered heteroalkylene, 2 to 6 membered heteroalkylene, or 2 to 4 membered heteroalkylene).

In embodiments, C¹ is R²⁹-substituted or unsubstituted cycloalkylene (e.g., C₃-C₈ cycloalkylene, C₃-C₆ cycloalkylene, or C₅-C₆ cycloalkylene). In embodiments, C¹ is R29-substituted cycloalkylene (e.g., C₃-C₈ cycloalkylene, C₃-C₆ cycloalkylene, or C₅-C₆ cycloalkylene). In embodiments, C¹ is an unsubstituted cycloalkylene (e.g., C₃-C₈ cycloalkylene, C₃-C₆ cycloalkylene, or C₅-C₆ cycloalkylene).

In embodiments, C¹ is R²⁹-substituted or unsubstituted heterocycloalkylene (e.g., 3 to 8 membered heterocycloalkylene, 3 to 6 membered heterocycloalkylene, or 5 to 6 membered heterocycloalkylene). In embodiments, C¹ is R²⁹-substituted heterocycloalkylene (e.g., 3 to 8 membered heterocycloalkylene, 3 to 6 membered heterocycloalkylene, or 5 to 6 membered heterocycloalkylene). In embodiments, C¹ is an unsubstituted heterocycloalkylene (e.g., 3 to 8 membered heterocycloalkylene, 3 to 6 membered heterocycloalkylene, or 5 to 6 membered heterocycloalkylene).

In embodiments, C¹ is R²⁹-substituted or unsubstituted arylene (e.g., C₆-C₁₀ arylene, C₁₀ arylene, or phenylene). In embodiments, C¹ is R²⁹-substituted arylene (e.g., C₆-C₁₀ arylene, C₁₀ arylene, or phenylene). In embodiments, C¹ is an unsubstituted arylene (e.g., C₆-C₁₀ arylene, C₁₀ arylene, or phenylene).

In embodiments, C¹ is R²⁹-substituted or unsubstituted heteroarylene (e.g., 5 to 10 membered heteroarylene, 5 to 9 membered heteroarylene, or 5 to 6 membered heteroarylene). In embodiments, C¹ is R²⁹-substituted heteroarylene (e.g., 5 to 10 membered heteroarylene, 5 to 9 membered heteroarylene, or 5 to 6 membered heteroarylene). In embodiments, C¹ is an unsubstituted heteroarylene (e.g., 5 to 10 membered heteroarylene, 5 to 9 membered heteroarylene, or 5 to 6 membered heteroarylene).

R²⁹ is independently oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHl₂, —OCHF₂, R³⁰-substituted or unsubstituted alkyl (e.g., C₁-C₈ alkyl, C₁-C₆ alkyl, or C₁-C₄ alkyl), R⁺-substituted or unsubstituted heteroalkyl (e.g., 2 to 8 membered heteroalkyl, 2 to 6 membered heteroalkyl, or 2 to 4 membered heteroalkyl), R³⁰-substituted or unsubstituted cycloalkyl (e.g., C₃-C₈ cycloalkyl, C₃-C₆ cycloalkyl, or C₅-C₆ cycloalkyl), R³⁰-substituted or unsubstituted heterocycloalkyl (e.g., 3 to 8 membered heterocycloalkyl, 3 to 6 membered heterocycloalkyl, or 5 to 6 membered heterocycloalkyl), R³⁰-substituted or unsubstituted aryl (e.g., C₆-C₁₀ aryl, C₁₀ aryl, or phenyl), or R³⁰-substituted or unsubstituted heteroaryl (e.g., 5 to 10 membered heteroaryl, 5 to 9 membered heteroaryl, or 5 to 6 membered heteroaryl).

In embodiments, R²⁹ is oxo, —OH, —NH₂, —COOH, or —CONH₂. In embodiments, R²⁹ is oxo.

In embodiments, L^{1C} is a bond, —O—, —S—, —NH—, —C(O)—, —C(O)O—, —C(O)NH—, R^{22C}-substituted or unsubstituted alkylene (e.g., C₁-C₈ alkylene, C₁-C₆ alkylene, or C₁-C₄ alkylene), R^{22C}-substituted or unsubstituted heteroalkylene (e.g., 2 to 8 membered heteroalkylene, 2 to 6 membered heteroalkylene, or 2 to 4 membered heteroalkylene), R^{22C}-substituted or unsubstituted cycloalkylene (e.g., C₃-C₈ cycloalkylene, C₃-C₆ cycloalkylene, or C₅-C₆ cycloalkylene), R^{22C}-substituted or unsubstituted heterocycloalkylene (e.g., 3 to 8 membered heterocycloalkylene, 3 to 6 membered heterocycloalkylene, or 5 to 6 membered heterocycloalkylene), R^{22C}-substituted or unsubstituted arylene (e.g., C₆-C₁₀ arylene, C₁₀ arylene, or phenylene), or R^{22C}-substituted or unsubstituted heteroarylene (e.g., 5 to 10 membered heteroarylene, 5 to 9 membered heteroarylene, or 5 to 6 membered heteroarylene). In embodiments, L^{1C} is a bond, —O—, —S—, —NH—, 1—C(O)—, —C(O)O—, —C(O)NH—, R^{22C}-substituted or unsubstituted alkenylene (e.g., C₂-C₈ alkenylene, C₂-C₆ alkenylene, or C₂-C₄ alkenylene). In embodiments, L^{1C} is a substituted or unsubstituted ethylenyl.

In embodiments, L^{2C} is a bond, —O—, —S—, —NH—, —C(O)—, —C(O)O—, —C(O)NH—, R^{23C}-substituted or unsubstituted alkylene (e.g., C₁-C₈ alkylene, C₁-C₆ alkylene, or C₁-C₄ alkylene), R^{23C}-substituted or unsubstituted heteroalkylene (e.g., 2 to 8 membered heteroalkylene, 2 to 6 membered heteroalkylene, or 2 to 4 membered heteroalkylene), R^{23C}-substituted or unsubstituted cycloalkylene (e.g., C₃-C₈ cycloalkylene, C₃-C₆ cycloalkylene, or C₅-C₆ cycloalkylene), R^{23C}-substituted or unsubstituted heterocycloalkylene (e.g., 3 to 8 membered heterocycloalkylene, 3 to 6 membered heterocycloalkylene, or 5 to 6 membered heterocycloalkylene), R^{23C}-substituted or unsubstituted arylene (e.g., C₆-C₁₀ arylene, C₁₀ arylene, or phenylene), or R^{23C}-substituted or unsubstituted heteroarylene (e.g., 5 to 10 membered heteroarylene, 5 to 9 membered heteroarylene, or 5 to 6 membered heteroarylene). In embodiments, L^{2C} is a bond, —O—, —S—, —NH—, —C(O)—, —C(O)O—, —C(O)NH—, R^{23C}-substituted or unsubstituted alkenylene (e.g., C₂-C₈ alkenylene, C₂-C₆ alkenylene, or C₂-C₄ alkenylene). In embodiments, L^{2C} is a substituted or unsubstituted ethylenyl.

In embodiments, (C(-L⁴-R⁴))_{z3} has the formula:

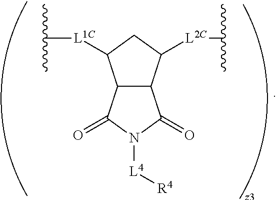

In embodiments, L^{1C} and L^{2C} are independently a bond, —O—, —S—, —NH—, —C(O)—, —C(O)O—, —C(O)NH—, substituted or unsubstituted alkylene, substituted or unsubstituted heteroalkylene

In embodiments, L⁴-R⁴ is a hydrophilic moiety. In embodiments, L⁴ is a hydrophilic moiety. In embodiments, R⁴ is a hydrophilic moiety.

In embodiments, L⁴ is a bond, —O—, —S—, —NH—, —C(O)—, —C(O)O—, —C(O)NH—, R¹⁵-substituted or unsubstituted alkylene (e.g., C₁-C₈ alkylene, C₁-C₆ alkylene, or C₁-C₄ alkylene), R¹⁵-substituted or unsubstituted heteroalkylene (e.g., 2 to 8 membered heteroalkylene, 2 to 6 membered heteroalkylene, or 2 to 4 membered heteroalkylene), R¹⁵-substituted or unsubstituted cycloalkylene (e.g., C₃-C₈ cycloalkylene, C₃-C₆ cycloalkylene, or C₅-C₆ cycloalkylene), R¹⁵-substituted or unsubstituted heterocycloalkylene (e.g., 3 to 8 membered heterocycloalkylene, 3 to 6 membered heterocycloalkylene, or 5 to 6 membered heterocycloalkylene), R¹⁵-substituted or unsubstituted arylene (e.g., C₆-C₁₀ arylene, C₁₀ arylene, or phenylene), or R¹⁵-substituted or unsubstituted heteroarylene (e.g., 5 to 10 membered heteroarylene, 5 to 9 membered heteroarylene, or 5 to 6 membered heteroarylene).

In embodiments, R⁴ is hydrogen, R¹⁶-substituted or unsubstituted alkyl (e.g., C₁-C₈ alkyl, C₁-C₆ alkyl, or C₁-C₄ alkyl), R¹⁶-substituted or unsubstituted heteroalkyl (e.g., 2 to 8 membered heteroalkyl, 2 to 6 membered heteroalkyl, or 2 to 4 membered heteroalkyl), R¹⁶-substituted or unsubstituted cycloalkyl (e.g., C₃-C₈ cycloalkyl, C₃-C₆ cycloalkyl, or C₅-C₆ cycloalkyl), R¹⁶-substituted or unsubstituted heterocycloalkyl (e.g., 3 to 8 membered heterocycloalkyl, 3 to 6 membered heterocycloalkyl, or 5 to 6 membered heterocycloalkyl), R¹⁶-substituted or unsubstituted aryl (e.g., C₆-C₁₀ aryl, C₁₀ aryl, or phenyl), or R¹⁶-substituted or unsubstituted heteroaryl (e.g., 5 to 10 membered heteroaryl, 5 to 9 membered heteroaryl, or 5 to 6 membered heteroaryl). In embodiments, R⁴ is a R¹⁶-substituted or unsubstituted heteroalkyl (e.g., 2 to 200 membered).

In embodiments, L⁴-R⁴ is a hydrophilic polymer. In embodiments, L⁴-R⁴ is oligoethylene glycol. In embodiments, L⁴-R⁴ is hexaethylene glycol. In embodiments, L⁴-R⁴ is pentaethylene glycol. In embodiments, L⁴-R⁴ is tetraethylene glycol. In embodiments, L⁴-R⁴ is polyethylene glycol. In embodiments, L⁴-R⁴ is polyethylene glycol having 2 to 100 ethylene oxide units. In embodiments, L⁴-R⁴ is polyethylene glycol having 2 to 50 ethylene oxide units. In embodiments, L⁴-R⁴ is polyethylene glycol having 2 to 30 ethylene oxide units. In embodiments, L⁴-R⁴ is polyethylene glycol having 2 to 20 ethylene oxide units. In embodiments, L⁴-R⁴ is polyethylene glycol having 2 to 10 ethylene oxide units. In embodiments, L⁴-R⁴ is polyethylene glycol having 5 to 100 ethylene oxide units. In embodiments, L⁴-R⁴ is polyethylene glycol having 5 to 50 ethylene oxide units. In embodiments, L⁴-R⁴ is polyethylene glycol having 5 to 30 ethylene oxide units. In embodiments, L⁴-R⁴ is polyethylene glycol having 5 to 25 ethylene oxide units. In embodiments, L⁴-R⁴ is polyethylene glycol having 5 to 20 ethylene oxide units. In embodiments, L⁴-R⁴ is polyethylene glycol having 5 to 15 ethylene oxide units. In embodiments, L⁴-R⁴ is polyethylene glycol having 5 to 10 ethylene oxide units.

In embodiments, L⁵ is a bond, —O—, —S—, —NH—, —C(O)—, —C(O)O—, —C(O)NH—, R¹⁸-substituted or unsubstituted alkylene (e.g., C₁-C₈ alkylene, C₁-C₆ alkylene, or C₁-C₄ alkylene), R¹⁸-substituted or unsubstituted heteroalkylene (e.g., 2 to 8 membered heteroalkylene, 2 to 6 membered heteroalkylene, or 2 to 4 membered heteroalkylene), R¹⁸-substituted or unsubstituted cycloalkylene (e.g., C₃-C₈ cycloalkylene, C₃-C₆ cycloalkylene, or C₅-C₆ cycloalkylene), R¹⁸-substituted or unsubstituted heterocycloalkylene (e.g., 3 to 8 membered heterocycloalkylene, 3 to 6 membered heterocycloalkylene, or 5 to 6 membered heterocycloalkylene), R¹⁸-substituted or unsubstituted arylene (e.g., C₆-C₁₀ arylene, C₁₀ arylene, or phenylene), or R¹⁸-substituted or unsubstituted heteroarylene (e.g., 5 to 10 membered heteroarylene, 5 to 9 membered heteroarylene, or 5 to 6 membered heteroarylene).

In embodiments, L⁵ is R¹⁸-substituted or unsubstituted alkylene (e.g., C₁-C₈ alkylene, C₁-C₆ alkylene, or C₁-C₄ alkylene). In embodiments, L⁵ is R¹⁸-substituted alkylene (e.g., C₁-C₈ alkylene, C₁-C₆ alkylene, or C₁-C₄ alkylene). In embodiments, L⁵ is an unsubstituted alkylene (e.g., C₁-C₈ alkylene, C₁-C₆ alkylene, or C₁-C₄ alkylene).

In embodiments, L⁵ is R¹⁸-substituted or unsubstituted heteroalkylene (e.g., 2 to 8 membered heteroalkylene, 2 to 6 membered heteroalkylene, or 2 to 4 membered heteroalkylene). In embodiments, L⁵ is R¹⁸-substituted heteroalkylene (e.g., 2 to 8 membered heteroalkylene, 2 to 6 membered heteroalkylene, or 2 to 4 membered heteroalkylene). In embodiments, L⁵ is an unsubstituted heteroalkylene (e.g., 2 to 8 membered heteroalkylene, 2 to 6 membered heteroalkylene, or 2 to 4 membered heteroalkylene).

In embodiments, L⁵ is R¹⁸-substituted or unsubstituted cycloalkylene (e.g., C₃-C₈ cycloalkylene, C₃-C₆ cycloalkylene, or C₅-C₆ cycloalkylene). In embodiments, L⁵ is R¹⁸-substituted cycloalkylene (e.g., C₃-C₈ cycloalkylene, C₃-C₆ cycloalkylene, or C₅-C₆ cycloalkylene). In embodiments, L⁵ is an unsubstituted cycloalkylene (e.g., C₃-C₈ cycloalkylene, C₃-C₆ cycloalkylene, or C₅-C₆ cycloalkylene).

In embodiments, L⁵ is R¹⁸-substituted or unsubstituted heterocycloalkylene (e.g., 3 to 8 membered heterocycloalkylene, 3 to 6 membered heterocycloalkylene, or 5 to 6 membered heterocycloalkylene). In embodiments, L⁵ is R¹⁸-substituted heterocycloalkylene (e.g., 3 to 8 membered heterocycloalkylene, 3 to 6 membered heterocycloalkylene, or 5 to 6 membered heterocycloalkylene). In embodiments, L⁵ is an unsubstituted heterocycloalkylene (e.g., 3 to 8 membered heterocycloalkylene, 3 to 6 membered heterocycloalkylene, or 5 to 6 membered heterocycloalkylene).

In embodiments, L⁵ is R¹⁸-substituted or unsubstituted arylene (e.g., C₆-C₁₀ arylene, C₁₀ arylene, or phenylene). In embodiments, L⁵ is R¹⁸-substituted arylene (e.g., C₆-C₁₀ arylene, C₁₀ arylene, or phenylene). In embodiments, L⁵ is an unsubstituted arylene (e.g., C₆-C₁₀ arylene, C₁₀ arylene, or phenylene).

In embodiments, L⁵ is R¹⁸-substituted or unsubstituted heteroarylene (e.g., 5 to 10 membered heteroarylene, 5 to 9 membered heteroarylene, or 5 to 6 membered heteroarylene). In embodiments, L⁵ is R¹⁸-substituted heteroarylene (e.g., 5 to 10 membered heteroarylene, 5 to 9 membered heteroarylene, or 5 to 6 membered heteroarylene). In embodiments, L⁵ is an unsubstituted heteroarylene (e.g., 5 to 10 membered heteroarylene, 5 to 9 membered heteroarylene, or 5 to 6 membered heteroarylene).

R¹⁸ is independently oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHl₂, —OCHF₂, R¹⁹-substituted or unsubstituted alkyl (e.g., C₁-C₈ alkyl, C₁-C₆ alkyl, or C₁-C₄ alkyl), R¹⁹-substituted or unsubstituted heteroalkyl (e.g., 2 to 8 membered heteroalkyl, 2 to 6 membered heteroalkyl, or 2 to 4 membered heteroalkyl), R¹⁹-substituted or unsubstituted cycloalkyl (e.g., C₃-C₈ cycloalkyl, C₃-C₆ cycloalkyl, or C₅-C₆ cycloalkyl), R¹⁹-substituted or unsubstituted heterocycloalkyl (e.g., 3 to 8 membered heterocycloalkyl, 3 to 6 membered heterocycloalkyl, or 5 to 6 membered heterocycloalkyl), R¹⁹-substituted or unsubstituted aryl (e.g., C₆-C₁₀ aryl, C₁₀ aryl, or phenyl), or R¹⁹-substituted or unsubstituted heteroaryl (e.g., 5 to 10 membered heteroaryl, 5 to 9 membered heteroaryl, or 5 to 6 membered heteroaryl).

In embodiments, R⁵ is hydrogen, —OH, —SH, —NH₂, —C(O)OH, —C(O)NH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or 5 to 6 membered heterocycloalkyl), R²⁰-substituted or unsubstituted aryl (e.g., C₆-C₁₀ aryl, C₁₀ aryl, or phenyl), R²⁰-substituted or unsubstituted heteroaryl (e.g., 5 to 10 membered heteroaryl, 5 to 9 membered heteroaryl, or 5 to 6 membered heteroaryl). In embodiments, R⁵ is a hydrophilic drug moiety, a targeting moiety or a detectable moiety. In embodiments, R⁵ is a hydrophilic drug moiety. In embodiments, R⁵ is a detectable moiety. In embodiments, R⁵ is a targeting moiety. In embodiments, R⁴ is not a drug moiety, targeting moiety or detectable moiety.

In embodiments, R⁵ is hydrogen, —OH, —SH, —NH₂, —C(O)OH, —C(O)NH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or 5 to 6 membered heterocycloalkyl), R²⁰-substituted or unsubstituted aryl (e.g., C₆-C₁₀ aryl, C₁₀ aryl, or phenyl), or R²⁰-substituted or unsubstituted heteroaryl (e.g., 5 to 10 membered heteroaryl, 5 to 9 membered heteroaryl, or 5 to 6 membered heteroaryl).

In embodiments, R⁵ is R²⁰-substituted or unsubstituted alkyl (e.g., C₁-C₈ alkyl, C₁-C₆ alkyl, or C₁-C₄ alkyl). In embodiments, R⁵ is R²⁰-substituted alkyl (e.g., C₁-C₈ alkyl, C₁-C₆ alkyl, or C₁-C₄ alkyl). In embodiments, R⁵ is an unsubstituted alkyl (e.g., C₁-C₈ alkyl, C₁-C₆ alkyl, or C₁-C₄ alkyl).

In embodiments, R⁵ is R²⁰-substituted or unsubstituted heteroalkyl (e.g., 2 to 8 membered heteroalkyl, 2 to 6 membered heteroalkyl, or 2 to 4 membered heteroalkyl). In embodiments, R⁵ is R²⁰-substituted heteroalkyl (e.g., 2 to 8 membered heteroalkyl, 2 to 6 membered heteroalkyl, or 2 to 4 membered heteroalkyl). In embodiments, R⁵ is an unsubstituted heteroalkyl (e.g., 2 to 8 membered heteroalkyl, 2 to 6 membered heteroalkyl, or 2 to 4 membered heteroalkyl).

In embodiments, R⁵ is R²⁰-substituted or unsubstituted cycloalkyl (e.g., C₃-C₈ cycloalkyl, C₃-C₆ cycloalkyl, or C₅-C₆ cycloalkyl). In embodiments, R⁵ is R²⁰-substituted cycloalkyl (e.g., C₃-C₈ cycloalkyl, C₃-C₆ cycloalkyl, or C₅-C₆ cycloalkyl). In embodiments, R⁵ is an unsubstituted cycloalkyl (e.g., C₃-C₈ cycloalkyl, C₃-C₆ cycloalkyl, or C₅-C₆ cycloalkyl).

In embodiments, R⁵ is R²⁰-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵ is R²⁰-substituted heterocycloalkyl (e.g., 3 to 8 membered heterocycloalkyl, 3 to 6 membered heterocycloalkyl, or 5 to 6 membered heterocycloalkyl). In embodiments, R⁵ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁵ is R²⁰-substituted or unsubstituted aryl (e.g., C₆-C₁₀ aryl, C₁₀ aryl, or phenyl). In embodiments, R⁵ is R²⁰-substituted aryl (e.g., C₆-C₁₀ aryl, C₁₀ aryl, or phenyl). In embodiments, R⁵ is an unsubstituted aryl (e.g., C₆-C₁₀ aryl, C₁₀ aryl, or phenyl). In embodiments, R⁵ is an unsubstituted phenyl.

In embodiments, R⁵ is R²⁰-substituted or unsubstituted heteroaryl (e.g., 5 to 10 membered heteroaryl, 5 to 9 membered heteroaryl, or 5 to 6 membered heteroaryl). In embodiments, R⁵ is R²⁰-substituted heteroaryl (e.g., 5 to 10 membered heteroaryl, 5 to 9 membered heteroaryl, or 5 to 6 membered heteroaryl). In embodiments, R⁵ is an unsubstituted heteroaryl (e.g., 5 to 10 membered heteroaryl, 5 to 9 membered heteroaryl, or 5 to 6 membered heteroaryl).

R²⁰ is independently oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHl₂, —OCHF₂, R²¹-substituted or unsubstituted alkyl (e.g., C₁-C₈ alkyl, C₁-C₆ alkyl, or C₁-C₄ alkyl), R²¹-substituted or unsubstituted heteroalkyl (e.g., 2 to 8 membered heteroalkyl, 2 to 6 membered heteroalkyl, or 2 to 4 membered heteroalkyl), R²¹-substituted or unsubstituted cycloalkyl (e.g., C₃-C₈ cycloalkyl, C₃-C₆ cycloalkyl, or C₅-C₆ cycloalkyl), R²¹-substituted or unsubstituted heterocycloalkyl (e.g., 3 to 8 membered heterocycloalkyl, 3 to 6 membered heterocycloalkyl, or 5 to 6 membered heterocycloalkyl), R²¹-substituted or unsubstituted aryl (e.g., C₆-C₁₀ aryl, C₁₀ aryl, or phenyl), or R²¹-substituted or unsubstituted heteroaryl (e.g., 5 to 10 membered heteroaryl, 5 to 9 membered heteroaryl, or 5 to 6 membered heteroaryl).

R⁸, R¹⁰, R¹¹, R¹², R¹³, R¹⁴, R¹⁵, R¹⁶, R¹⁹, R²¹, R^{22A}, R^{23A}, R^{22B}, R^{23B}, R^{22C}, R^{23C}, R²⁶, R²⁸, and R³ are independently oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHl₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, L^{1A}, L^{2A}, L^{1B}, L^{2B}, L^{1C}, and L^{2C} are independently a bond or a substituted or unsubstituted alkylene. In embodiments, L^{1A}, L^{2A}, L^{1B}, L^{2B}, L^{1C}, and L^{2C} are independently a bond or a substituted or unsubstituted alkenylene. In embodiments, L^{1A}, L^{2A}, L^{1B}, L^{2B}, L^{1C}, and L^{2C} are independently a substituted or unsubstituted alkenylene. In embodiments, L^{1A}, L^{2A}, L^{1B}, L^{2B}, L^{1C}, and L^{2C} are independently unsubstituted alkenylene.

In embodiments, L^{1A}, L^{2A}, L^{1B}, L^{2B}, L^{1C}, and L^{2C} are independently a bond or a substituted or unsubstituted alkylene (e.g., C₁-C₂₀ alkylene, C₁-C₁₈ alkylene, C₁-C₁₂ alkylene, C₁-C₁₀ alkylene, C₁-C₈ alkylene, C₁-C₆ alkylene, C₁-C₄ alkylene, or C₁-C₂ alkylene). In embodiments, L^{1A}, L^{2A}, L^{1B}, L^{2B}, L^{1C}, and L^{2C} are independently a bond or a substituted or unsubstituted alkenylene (e.g., C₂-C₂₀ alkenylene, C₂-C₁₈ alkenylene, C₂-C₁₂ alkenylene, C₂-C₁₀ alkenylene, C₂-C₈ alkenylene, C₂-C₆ alkenylene, C₂-C₄ alkenylene, or C₂ alkenylene). In embodiments, L^{1A}, L^{2A}, L^{1B}, L^{2B}, L^{1C}, and L^{2C} are independently a substituted or unsubstituted alkenylene (e.g., C₂-C₂₀ alkenylene, C₂-C₁₈ alkenylene, C₂-C₁₂ alkenylene, C₂-C₁₀ alkenylene, C₂-C₈ alkenylene, C₂-C₆ alkenylene, C₂-C₄ alkenylene, or C₂ alkenylene). In embodiments, L^{1A}, L^{2A}, L^{1B}, L^{2B}, L^{1C}, and L^{2C} are independently an unsubstituted alkenylene (e.g., C₂-C₂₀ alkenylene, C₂-C₁₈ alkenylene, C₂-C₁₂ alkenylene, C₂-C₁₀ alkenylene, C₂-C₈ alkenylene, C₂-C₆ alkenylene, C₂-C₄ alkenylene, or C₂ alkenylene).

In embodiments, the polymeric micellar nanoparticle is substantially free of gadolinium (Gd). In embodiments, the polymeric micellar nanoparticle is substantially free of Gd³⁺. In embodiments, the polymeric micellar nanoparticle is substantially free of Gd₂O₃. In embodiments, the term “substantially free of gadolinium” or similar phrase means that gadolinium is not explicitly introduced into the polymeric micellar nanoparticle during synthesis. Where a nanoparticle is substantially free of Gd, a person having ordinary skill will understand that the nanoparticle may have no Gd, no measurable amount of Gd or only trace amounts of Gd. In embodiments, the polymeric micellar nanoparticle is free of gadolinium (Gd). In embodiments, the polymeric micellar nanoparticle is free of Gd³⁺. In embodiments, the polymeric micellar nanoparticle is free of Gd₂O₃. In embodiments, the term “free of gadolinium” or similar phrase means that no detectable amounts of gadolinium are present.

In embodiments, the polymeric micellar nanoparticle is prepared from amphiphilic block copolymers, as disclosed herein. In embodiments, the amphiphilic block copolymer is an amphiphilic tri-block copolymer (e.g., includes three blocks).

In embodiments, the polymeric micellar nanoparticle is a spherical polymeric micellar nanoparticle. In embodiments, the polymeric micellar nanoparticle is a cylindrical polymeric micellar nanoparticle.

In embodiments, the polymeric micellar nanoparticle has a diameter of about 10 nm to about 1000 nm (e.g., about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nm).

In embodiments, the spherical polymeric micellar nanoparticle has a diameter of about 10 nm to about 300 nm (e.g., about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 nm). In embodiments, the spherical polymeric micellar nanoparticle has a diameter of about 30 nm.

In embodiments, the cylindrical polymeric micellar nanoparticle has a diameter of about 10 nm to about 1000 nm (e.g., about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nm). In embodiments, the cylindrical polymeric micellar nanoparticle has a diameter of about 25 nm.

In embodiments, the cylindrical polymeric micellar nanoparticle has a cylindrical length of about 100 nm or longer (e.g., about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 nm or longer).

In embodiments, the cylindrical polymeric micellar nanoparticle has a cylindrical length of about 1 μm or longer (e.g., about 1000, 1100, 1200, 1300, 1400, 1500, 1600, 1700, 1800, 1900, 2000 nm or longer).

In embodiments, the polymeric micellar nanoparticle includes a block copolymer including the following structure:

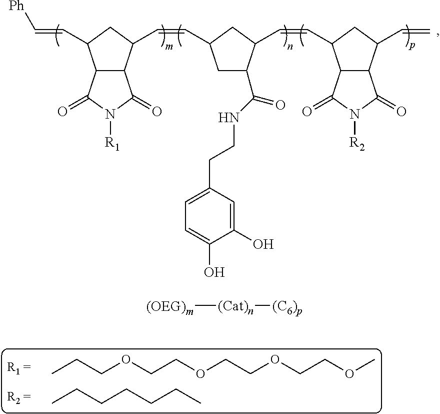

where m=38, n=34, and p=50 or where m=20, n=23, and p=43.

In embodiments, z1, z2 and z3 are independently integers from 10-35. In embodiments, z1, z2 and z3 are independently integers from 20-50. In embodiments, z1 is an integer from 1 to 100. In embodiments, z1 is an integer from 2 to 50. In embodiments, z1 is an integer from 20 to 100. In embodiments, z1 is an integer from 20 to 50. In embodiments, z2 is an integer from 1 to 100. In embodiments, z2 is an integer from 2 to 50. In embodiments, z2 is an integer from 20 to 100. In embodiments, z2 is an integer from 20 to 50. In embodiments, z3 is an integer from 1 to 100. In embodiments, z3 is an integer from 2 to 50. In embodiments, z3 is an integer from 20 to 100. In embodiments, z3 is an integer from 20 to 50.

In embodiments, z1 is an integer from 40 to 50, z2 is from 20 to 40, and z3 is an integer from 15 to 40. In embodiments, z1 is an integer from 40 to 50, z2 is from 20 to 40, and z3 is an integer from 20 to 40. In embodiments, z1 is an integer from 43 to 50, z2 is from 20 to 40, and z3 is an integer from 15 to 40. In embodiments, z1 is an integer from 43 to 50, z2 is from 23 to 40, and z3 is an integer from 15 to 40. In embodiments, z1 is an integer from 43 to 50, z2 is from 23 to 40, and z3 is an integer from 20 to 40. In embodiments, z1 is an integer from 43 to 50, z2 is from 23 to 34, and z3 is an integer from 20 to 38.

In embodiments, z1 is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In embodiments, z2 is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In embodiments, z3 is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In embodiments, z5 is 1. In embodiments, z5 is 0.

In embodiments, the targeting moiety is an antibody, peptide sequence (e.g. peptide sequence capable of recognizing receptors such as an integrin), nucleic acid, or a small molecule (e.g., small molecule capable of binding a folic acid receptor). In embodiments, the targeting moiety is targets biochemical markers (e.g., proteins).

In embodiments, the polymeric micellar nanoparticle has a relaxivity value (r₁) of about 7×10⁻³ M⁻¹ S⁻¹ to about 10×10⁻³ M⁻¹ S⁻¹ (e.g., about 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10×10⁻³ M⁻¹ S⁻¹). Relaxivity, r₁, arises from metal-bound and/or proximate hydrogen-bonded water molecules, dipolarly interacting with the unpaired electrons of the metal ion:

r₁=f_{M}×(T_{1M}+τ_{M})⁻¹+ᵣ₁^{os} (1)

where f_{M} is the mole fraction of interacting water molecules, T_{1M} their proton relaxation time due to the paramagnetic Fe(III) ion, τ_{M} the exchange lifetime and r₁^{os} the contribution of outer-sphere fast diffusing water protons. T_{1M} depends on r⁶, the distance of the interacting dipoles, on the correlation time (τ_{C}) for the proton-electron dipolar interaction and on ω_{I} and ω_{S}, the proton and electron Larmor frequencies, respectively. In other words, r₁(r₂) value was calculated after the curve fitting of 1/T₁ (s⁻¹) (or 1/T₂(s⁻¹)) versus Fe(III) ions concentrations (μM), where both of the longitudinal and transverse relaxation time (T₁ and T₂) measurements were acquired with a contrast agent analyzer (a Time Domain-NMR instrument, Bruker, Minispec mq60, 1.41T/60 MHz, 37° C.).

In embodiments, the polymeric micellar nanoparticle described herein may include one or more additional detectable moieties. In embodiments, the polymeric micellar nanoparticle described herein does not include one or more additional detectable moieties. A “detectable moiety” as used herein refers to a moiety that can be covalently or noncovalently attached to a compound or biomolecule that can be detected for instance, using techniques known in the art. In embodiments, the detectable moiety is covalently attached. The detectable moiety may provide for imaging of the attached compound or biomolecule. The detectable moiety may indicate the contacting between two compounds. Exemplary detectable moieties are fluorophores, antibodies, reactive dyes, radio-labeled moieties, magnetic contrast agents, and quantum dots. Exemplary fluorophores include fluorescein, rhodamine, GFP, coumarin, FITC, Alexa fluor, Cy3, Cy5, BODIPY, and cyanine dyes. Exemplary radionuclides include Fluorine-18, Gallium-68, and Copper-64.

Unless indicated to the contrary, the terms “nuclear magnetic resonance imaging,” “magnetic resonance imaging” and the like are used synonymously herein in the usual and customary sense. The term “contrast agent,” “contrast agent for nuclear magnetic resonance imaging,” “MRI contrast agent” and the like refer, in the usual and customary sense, to a contrast medium useful to improve the visibility of internal structures (e.g., organs, vessels, metabolite, and the like) in MRI. Without wishing to be bound by any theory, it is believed that MRI contrast agents modify the relaxation times of atoms within tissues subject to MRI. In many embodiments, the relaxation time is shortened. In some embodiments, the relaxation is increased. Modulation of the relaxation time is experimentally observed to alter the contrast in the MRI image.

In embodiments, the polymeric micellar nanoparticle provided herein has a higher stability relative the contrast agents known in the art (e.g., Gd-based contrast agents). Without wishing to be bound by any theory, the stability of the contrast agent described herein can be examined in one or two of the following respects: (1) no collapse or change of the micellar nanoparticle morphologies is observed over time; and (2) the total metal ion content and/or relaxivity value of the Fe(III)-chelated nanoparticles does not change significantly over time. In embodiments, no collapse or change of the micellar nanoparticle morphologies is observed in the contrast agent provided herein over a period of about 1, 2, 3, 4, 5, 6, 7, 8, 9, 10, 11, 12, 13, 14, 15, 16, 17, 18, 19, 20, 21, 22, 23 hours or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days or longer. In embodiments, the total metal ion content of the Fe(III)-chelated nanoparticles changes less than about 10%, 9%, 8%, 7%, 6%, 5%, 4%, 3%, 2%, 1%, 0.5%, 0.1% or less over time (e.g., over a period of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days or longer). In embodiments, the relaxivity value of the Fe(III)-chelated nanoparticles changes less than about 10%, 9%, 8%, 7%, 6%, 5%, 4%, 3%, 2%, 1%, 0.5%, 0.1% or less over time (e.g., over a period of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days or longer).

In embodiments, the polymeric micellar nanoparticle provided herein is less toxic than the contrast agents known in the art (e.g., Gd-based contrast agents). In embodiments, the polymeric micellar nanoparticle provided herein does not induce necrosis and/or apoptosis of cells that are exposed to the contrast agent. In embodiments, the polymeric micellar nanoparticle provided herein induces necrosis and/or apoptosis of cells that are exposed to the contrast agent to the extent of less than about 1%, 0.9%, 0.8%, 0.7%, 0.6%, 0.5%, 0.4%, 0.3%, 0.2%, 0.1%, 0.05%, 0.01% or less of the cells that are exposed to the contrast agent. In embodiments, the polymeric micellar nanoparticle provided herein does not cause toxic symptoms that can be caused by Gd-based contrast agents. The toxic symptoms may include, but are not limited to,
Pain—aching; burning, tingling, and/or prickling pain (paresthesia); deep bone pain. Typically in extremities or joints, and sometimes in the location where the MRI occurred, like the head.
Dermal changes—like tight skin, lesions, hyperpigmentation. Most often in extremities.
Muscle issues—twitching—small, local, rapid contractions and weakness
Ocular problems—worsening vision, dry eyes, bloodshot eyes
Cognitive symptoms
Ear, nose and throat—tinnitus, swallowing, and voice problems
Low body temperature
Hair loss
Itchy skin
Balance problems or
Swelling of extremities (edema)

In embodiments, the polymeric micellar nanoparticle provided herein is further combined with a pharmaceutically acceptable excipient as a diagnostic pharmaceutical composition.

A “pharmaceutical composition” is a formulation containing the polymeric micellar nanoparticle described herein in a form suitable for administration to a subject. In embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a polymeric micellar nanoparticle) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In embodiments, the active contrast agent is mixed under sterile conditions with a pharmaceutically acceptable carrier, and with any preservatives, buffers, or propellants that are required

In embodiments, exemplary pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

In another aspect is an aqueous pharmaceutical composition including the polymeric micelle as described herein and a pharmaceutically acceptable excipient. In embodiments, the pharmaceutical composition is a parental dosage form. In embodiments, the pharmaceutical composition is an intravenous dosage form. In embodiments, the pharmaceutical composition is an intramyocardial dosage form.

In embodiments, the pharmaceutical composition is isotonic. In embodiments, the pharmaceutical composition is isotonic to human blood. The pharmaceutical composition may have a pH from about 3.5 to about 6.2. In embodiments, the pharmaceutical composition is isotonic. In embodiments, the pharmaceutical composition is isotonic and has a pH from about 3.5 to about 6.2. In embodiments, the pharmaceutical composition is isotonic and has a pH from about 4.0 to about 6.2. In embodiments, the pharmaceutical composition is isotonic and has a pH from about 4.5 to about 6.2. In embodiments, the pharmaceutical composition is isotonic and has a pH from about 5.0 to about 6.2. In embodiments, the pharmaceutical composition is isotonic and has a pH from about 5.5 to about 6.2.

In embodiments, the pharmaceutical composition has a pH from about 3.5 to about 6.2. In embodiments, the pharmaceutical composition has a pH from about 4.0 to about 6.2. In embodiments, the pharmaceutical composition has a pH from about 4.5 to about 6.2. In embodiments, the pharmaceutical composition has a pH from about 5.0 to about 6.2. In embodiments, the pharmaceutical composition has a pH from about 5.5 to about 6.2.

In embodiments, the pharmaceutical composition has a pH from about 3.5 to about 8.2. In embodiments, the pharmaceutical composition has a pH from about 4.0 to about 8.2. In embodiments, the pharmaceutical composition has a pH from about 4.5 to about 8.2. In embodiments, the pharmaceutical composition has a pH from about 5.0 to about 8.2. In embodiments, the pharmaceutical composition has a pH from about 5.5 to about 8.2. In embodiments, the pharmaceutical composition has a pH from about 6.0 to about 8.2. In embodiments, the pharmaceutical composition has a pH from about 6.5 to about 8.2. In embodiments, the pharmaceutical composition has a pH from about 7.0 to about 8.2. In embodiments, the pharmaceutical composition has a pH from about 7.5 to about 8.2. In embodiments, the pharmaceutical composition has a pH from about 7.0 to about 8.0. In embodiments, the pharmaceutical composition has a pH about 7.8.

### III. METHODS

In another aspect, there is provided a method of providing visibility of an internal body structure of a subject undergoing MRI. The method includes administering to the subject an effective amount of a polymeric micellar nanoparticle described herein. The internal body structure includes, but is not limited to, tissues, nerves, muscles, organs, vessels, blood, extracellular fluid, and metabolites.

In an aspect, is provided a method of detecting an internal body structure of a subject, the method comprising administering to said subject an effective amount of the polymeric micellar nanoparticle as described herein and detecting said polymeric micellar nanoparticle using magnetic resonance imaging thereby detecting said internal body structure.

In embodiments, the effective amount refers to an amount that is sufficient to enhance the contrast of MRI by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or more compared to the contrast level provided by a similar amount of other contrast agents.

In embodiments, the effective amount refers to an amount that is sufficient to enhance the contrast of MRI by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or more compared to the contrast level in the absence of the polymeric micellar nanoparticle as described herein.

In embodiments, the method may further include detecting the polymeric micellar nanoparticle.

In embodiments, the method may further include administering an additional contrast agent to the subject. In embodiments, the additional contrast agent may be an intravenous contrast agent, an intravascular contrast agent, an organ specific agent, a pH-sensitive agent, a responsive (or bioactivated) agent or a Gd-based contrast agent, such as, for example, gadoterate (Dotarem), gadodiamide (Omniscan), gadobenate (MultiHance), gadopentetate (Magnevist), gadoteridol (ProHance), gadofosveset (Ablavar, formerly Vasovist), gadoversetamide (OptiMARK), gadoxetate (Eovist), or gadobutrol (Gadavist).

“Co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation).

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

Examples of routes of administration include parenteral, e.g., intravenous, intradermal, subcutaneous, oral (e.g., inhalation), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

In another aspect, there is provided a method for synthesizing a polymeric micellar nanoparticle. The method includes functionalizing a tri-block copolymer amphiphile to incorporate a catechol group in the middle block of the block copolymer amphiphile, thereby providing a functionalized tri-block copolymer amphiphile. The method further includes contacting the functionalized tri-block copolymer amphiphile under conditions suitable to afford a catechol-containing tri-block copolymer. The method further includes contacting the catechol-containing tri-block copolymer with a metal salt, thereby providing the polymeric micellar nanoparticle.

In another aspect, there is provided a method for synthesizing a polymeric micellar nanoparticle. The method includes functionalizing (e.g., reacting) a block copolymer amphiphile to incorporate a catechol group (e.g., 1,2-benzenediol) in one block of the block copolymer amphiphile, thereby providing a functionalized block copolymer amphiphile. The method further includes contacting the functionalized block copolymer amphiphile under conditions suitable to afford a catechol-containing block copolymer. The method further includes contacting the catechol-containing block copolymer with a metal salt, thereby providing the polymeric micellar nanoparticle.

In embodiments, the metal is iron (e.g., Fe(III)). In embodiments, the metal is Mn²⁺, Mn³⁺, Fe³⁺, or Cu²⁺. In embodiments, the metal is Fe³⁺, alternatively written as Fe(III).

In embodiments, the block copolymer amphiphile is a tri-block copolymer amphiphile and the catechol group (e.g., 1,2-benzenediol) is incorporated to the middle block (e.g., second block) of the tri-block copolymer amphiphile.

In embodiments, without wishing to be bound by any theory, a polymeric micellar nanoparticle (e.g., (OEG)₃₈-(Cat)₃₄-(C₆)₅₀ (Polymer 1)) is synthesized as follows: (OEG)₃₈-(NHS)₃₄-(C₆)₅₀, 3-hydroxytyramine hydrochloride, and N,N-Diisopropylethylamine are fully dissolved in DMF. The mixture is stirred at room temperature for a period of time. The solution is then precipitated into HCl solution multiple times. The brown precipitate is filtered off and redissolved in THF. The solution is precipitated into cold ether multiple times. The final product is collected and dried under vacuum overnight to afford a brown powder as Polymer 1.

In embodiments, without wishing to be bound by any theory, a polymeric micellar nanoparticle (e.g., (OEG)₃₈-(Cat)₃₄-(C₆)₅₀ (Polymer 1)) is synthesized as follows: (OEG)₃₈-(NHS)₃₄-(C₆)₅₀ (M_{n, SEC}=33.7 kg/mol, 300 mg, 0.0089 mmol), 3-hydroxytyramine hydrochloride (287 mg, 1.51 mmol), and N,N-Diisopropylethylamine (390 mg, 3.02 mmol) are fully dissolved in 10 mL of DMF. The mixture is stirred at room temperature for about 2 days. The solution is then precipitated into 1M HCl solution three times. The brown precipitate is filtered off and redissolved in 10 mL THF. The solution is precipitated into cold ether three times. The final product is collected and dried under vacuum overnight to afford a brown powder (268 mg; Yield: 86%) as Polymer 1.

In embodiments, without wishing to be bound by any theory, a polymeric micellar nanoparticle (e.g., (OEG)₂₀-(Cat)₂₃-(C₆)₄₃ (Polymer 2)) is synthesized as follows: (OEG)₂₀-(NHS)₂₃-(C₆)₄₃, 3-hydroxytyramine hydrochloride, and N,N-Diisopropylethylamine are fully dissolved in DMF. The mixture is stirred at room temperature for a period of time (e.g., 2 days). The solution is then precipitated into HCl solution several times. The brown precipitation is filtered off, redissolved into THF then precipitated into cold ether several times. The final product is collected and dried under vacuum overnight to afford a brown powder as Polymer 2.

In embodiments, without wishing to be bound by any theory, a polymeric micellar nanoparticle (e.g., (OEG)₂₀-(Cat)₂₃-(C₆)₄₃ (Polymer 2)) is synthesized as follows: (OEG)₂₀-(NHS)₂₃-(C₆)₄₃ (M_{n, SEC}=23.1 kg/mol, 180 mg, 0.0078 mmol), 3-hydroxytyramine hydrochloride (155 mg, 0.82 mmol), and N,N-Diisopropylethylamine (211 mg, 1.64 mmol) are fully dissolved in 10 mL of DMF. The mixture is stirred at room temperature for about 2 days. The solution is then precipitated into 1M HCl solution three times. The brown precipitation is filtered off, redissolved into 8 mL THF then precipitated into cold ether three times. The final product is collected and dried under vacuum overnight to afford a brown powder (153 mg; Yield: 82%) as Polymer 2.

### IV. KITS

In another aspect, there is provided a kit including a contrast agent (e.g., a polymeric micellar nanoparticle) described herein. In embodiments, the contrast agent described herein may, if desired, be presented in a kit (e.g., a pack, a storage vessel or a container) which may contain one or more unit dosage forms containing the contrast agent.

The pack may for example include metal or plastic foil, such as a blister pack. The pack or the container may be accompanied by instructions for administration. Compositions comprising a contrast agent described herein that is formulated in a compatible pharmaceutical carrier may also be prepared, placed in an appropriate container, and labeled for use of an indicated purpose (e.g., for MRI imaging). Instructions for use may also be provided.

### V. EMBODIMENTS

Additional embodiments of the compositions and methods disclosed herein include Embodiments P1 to P7 following.

Embodiment P1. A contrast agent for nuclear magnetic resonance imaging, comprising a polymeric micellar nanoparticle, said polymeric micellar nanoparticle comprising a Fe(III)-catecholate complex.

Embodiment P2. The contrast agent according to embodiment P1, wherein said contrast agent is free of Gd.

Embodiment P3. The contrast agent according to embodiment P1, wherein said polymeric micellar nanoparticle is prepared from amphiphilic tri-block copolymers.

Embodiment P4. The contrast agent according to embodiment P1, wherein said polymeric micellar nanoparticle is a spherical polymeric micellar nanoparticle.

Embodiment P5. The contrast agent according to embodiment P1, wherein said polymeric micellar nanoparticle is a cylindrical polymeric micellar nanoparticle.

Embodiment P6. A method for synthesizing a polymeric micellar nanoparticle, the method including: functionalizing a tri-block copolymer amphiphile to incorporate a catechol group in the middle block of said tri-block copolymer amphiphile, thereby providing a functionalized tri-block copolymer amphiphile; contacting said functionalized tri-block copolymer amphiphile under conditions suitable to afford a catechol-containing block copolymer; and contacting said catechol-containing block copolymer with a metal salt, thereby providing said polymeric micellar nanoparticle.

Embodiment P7. A diagnostic pharmaceutical composition including a polymeric micellar nanoparticle as set forth in embodiment P1, in combination with a pharmaceutically acceptable excipient.

Further embodiments include Embodiments 1 to 18 following.

Embodiment 1. A composition including a polymeric micellar nanoparticle, wherein the polymeric micellar nanoparticle includes a Fe(III)-catecholate complex.

Embodiment 2. The composition of embodiment 1, wherein the Fe(III)-catecholate complex is covalently linked to the polymeric micellar nanoparticle.

Embodiment 3. The composition of embodiment 1 or embodiment 2, wherein the composition is free of gadolinium (Gd).

Embodiment 4. The composition of any of embodiments 1 to 3, wherein the polymeric micellar nanoparticle is prepared from amphiphilic block copolymers.

Embodiment 5. The composition of embodiment 4, wherein the amphiphilic block copolymer is an amphiphilic tri-block copolymer.

Embodiment 6. The composition of any of embodiments 1 to 5, wherein the polymeric micellar nanoparticle is a spherical polymeric micellar nanoparticle.

Embodiment 7. The composition of any of embodiments 1 to 5, wherein the polymeric micellar nanoparticle is a cylindrical polymeric micellar nanoparticle.

Embodiment 8. The composition of any of embodiments 1 to 7, wherein the polymeric micellar nanoparticle has a diameter of about 10 nm to about 1000 nm.

Embodiment 9. The composition of any of embodiments 1 to 8, wherein the polymeric micellar nanoparticle has a relaxivity value (r1) of about 7×10⁻³ M⁻¹ s⁻¹ to about 10×10⁻³ M⁻¹ s⁻¹.

Embodiment 10. The composition of any of embodiments 1 to 9, further including a detectable moiety.

Embodiment 11. A contrast agent for magnetic resonance imaging (MRI) including the composition of any of embodiments 1 to 10.

Embodiment 12. A method of providing visibility of an internal body structure of a subject undergoing MRI, the method including administering to the subject an effective amount of the contrast agent of embodiment 11 or the composition of any one of embodiments 1 to 8.

Embodiment 13. The method of embodiment 12, further including administering an additional contrast agent to the subject.

Embodiment 14. A method for synthesizing a polymeric micellar nanoparticle, the method comprising: functionalizing a block copolymer amphiphile to incorporate a catechol group in one block of the block copolymer amphiphile, thereby providing a functionalized block copolymer amphiphile; contacting the functionalized block copolymer amphiphile under conditions suitable to afford a catechol-containing block copolymer; and contacting the catechol-containing block copolymer with a metal salt, thereby providing the polymeric micellar nanoparticle.

Embodiment 15. The method of embodiment 14, wherein the metal is an iron.

Embodiment 16. The method of embodiment 14 or 15, wherein the block copolymer amphiphile is a tri-block copolymer amphiphile.

Embodiment 17. A diagnostic pharmaceutical composition including the composition of embodiment 1 and a pharmaceutically acceptable excipient.

Embodiment 18. A kit comprising an instruction and the composition of any one of embodiments 1 to embodiment 10 or the contrast agent of embodiment 11.

Additional Embodiments.

Embodiment 19. A polymeric micellar nanoparticle, wherein the polymeric micellar nanoparticle includes a Fe(III)-catecholate complex.

Embodiment 20. The polymeric micellar nanoparticle of embodiment 19, wherein the polymeric micellar nanoparticle includes a plurality of block copolymers, wherein each block copolymer includes a first block of hydrophobic polymerized monomers, a second block of catechol polymerized monomers and a third block of hydrophilic polymerized monomers, wherein:
(i) the first block of hydrophobic polymerized monomers include a hydrophobic moiety covalently attached to each first block monomer backbone moiety within the first block of hydrophobic polymerized monomers, wherein each hydrophobic moiety is optionally different;
(ii) the second block of catechol polymerized monomers include a catechol moiety covalently attached to each second block monomer backbone moiety within the second block of catechol polymerized monomers, wherein at least one catechol moiety is complexed to iron thereby forming the Fe(III)-catecholate complex; and
(iii) the third block of hydrophilic polymerized monomers include a hydrophilic moiety covalently attached to each third block monomer backbone moiety within the third block of hydrophilic polymerized monomers, wherein each hydrophilic moiety is optionally different.

Embodiment 21. The polymeric micellar nanoparticle of embodiment 20, wherein each of the hydrophobic moieties are the same.

Embodiment 22. The polymeric micellar nanoparticle of embodiment 20 of the formula:

R¹L¹-(A(-L²-R²))_{z1}-(B(-L³-R³))_{z2}-[(C(-L⁴-R⁴))_{z3}]_{z5}-L⁵-R⁵

wherein
(A(-L²-R²))_{z1} is the first block of hydrophobic polymerized monomers;
(B(-L³-R³))_{z2} is the second block of catechol polymerized monomers;
(C(-L⁴-R⁴))_{z3} is the third block of hydrophilic polymerized monomers;
A is a first block monomer backbone moiety;
B is a second block monomer backbone moiety;
C is a third block monomer backbone moiety;
z1, z2 and z3 are independently integers from 1 to 100;
z5 is 0 or 1;
L¹ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
L²-R² is a hydrophobic moiety, wherein
   L² is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
   R² is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
L³-R³ is a catechol moiety, wherein
   L³ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
   R³ is substituted or unsubstituted 1,2-dihydroxyphenyl;
L⁴-R⁴ is a hydrophilic moiety, wherein
   L⁴ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
   R⁴ is —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
L⁵ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R⁵ is —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

Embodiment 23. The polymeric micellar nanoparticle of embodiment 22, wherein R³ is unsubstituted 1,2-dihydroxyphenyl.

Embodiment 24. The polymeric micellar nanoparticle of embodiments 22 or 23, wherein L³-R³ is:

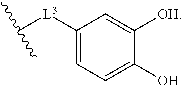

Embodiment 25. The polymeric micellar nanoparticle of any one of embodiments 22 to 24, wherein L³ is —C(O)—N(H)—(CH₂)_{z4}- wherein z4 is 1 to 10.

Embodiment 26. The polymeric micellar nanoparticle of embodiment 25 wherein z4 is 1 to 4.

Embodiment 26. The polymeric micellar nanoparticle of embodiment 25 wherein z4 is 1 or 2.

Embodiment 27. The polymeric micellar nanoparticle of embodiment 25 wherein z4 is 2.

Embodiment 28. The polymeric micellar nanoparticle of any one of embodiments 22 to 27, wherein z1, z2 and z3 are independently integers from 10-35.

Embodiment 29. The polymeric micellar nanoparticle of any one of embodiments 22 to 27, wherein z1, z2 and z3 are independently integers from 20-50.

Embodiment 30. The polymeric micellar nanoparticle of any one of embodiments 22 to 29, wherein (A(-L²-F²))_{z1} has the formula (-L^{1A}-A¹(-L²-R²)-L^{2A}-)_{z1}, wherein L^{1A} and L^{2A} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; A¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 31. The polymeric micellar nanoparticle of any one of embodiments 22 to 30, wherein (B(-L³-R³))_{z2} has the formula (-L^{1B}-B¹(-L³-R³)-L^{2B}-)_{z2}, wherein L^{1B} and L^{2B} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; B¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 32. The polymeric micellar nanoparticle of any one of embodiments 22 to 31, wherein (C(-L⁴-R⁴)) has the formula (-L^{1C}-C¹(-L⁴-F⁴)-L^{2C}-)_{z3}, wherein L^{1C} and L^{2C} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; C¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 33. The polymeric micellar nanoparticle of any one of embodiments 22 to 32, wherein (B(-L³-R³))_{z2} has the formula:

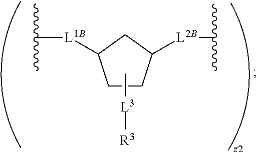

wherein L^{1B} and L^{2B} are independently a bond, —O—, —S—, —NH—, —C(O)—, —C(O)O—, —C(O)NH—, substituted or unsubstituted alkylene, substituted or unsubstituted heteroalkylene.

Embodiment 34. The polymeric micellar nanoparticle of any one of embodiments 22 to 33, wherein (A(-L²-R²))_{z1} has the formula:

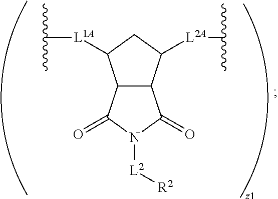

wherein L^{1A} and L^{2A} are independently a bond, —O—, —S—, —NH—, —C(O)—, —C(O)O—, —C(O)NH—, substituted or unsubstituted alkylene, substituted or unsubstituted heteroalkylene.

Embodiment 35. The polymeric micellar nanoparticle of any one of embodiments 22 to 34, wherein (C(-L⁴-R⁴))_{z3} has the formula:

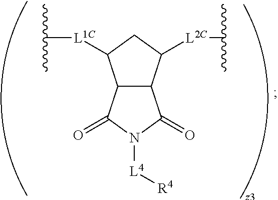

wherein L^{1C} and L^{2C} are independently a bond, —O—, —S—, —NH—, —C(O)—, —C(O)O—, —C(O)NH—, substituted or unsubstituted alkylene, substituted or unsubstituted heteroalkylene.

Embodiment 36. The polymeric micellar nanoparticle of any one of embodiments 22 to 35, wherein L²-R² is substituted or unsubstituted alkyl.

Embodiment 37. The polymeric micellar nanoparticle of any one of embodiments 22 to 35, wherein L²-R² is unsubstituted alkyl.

Embodiment 38. The polymeric micellar nanoparticle of any one of embodiments 22 to 35, wherein L²-R² is unsubstituted C₁-C₁₀ alkyl.

Embodiment 39. The polymeric micellar nanoparticle of any one of embodiments 22 to 35, wherein L²-R² is

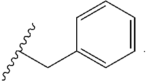 [SMILES: *Cc1ccccc1]

Embodiment 40. The polymeric micellar nanoparticle of any one of embodiments 22 to 39, wherein L⁴-R⁴ is a hydrophilic polymer.

Embodiment 41. The polymeric micellar nanoparticle of any one of embodiments 22 to 39, wherein L⁴-R⁴ is a polyethylene glycol.

Embodiment 42. The polymeric micellar nanoparticle of any one of embodiments 22 to 39, wherein L⁴-R⁴ is polyethylene glycol having 2 to 100 ethylene oxide units.

Embodiment 43. The polymeric micellar nanoparticle of any one of embodiments 22 to 39, wherein L⁴-R⁴ is polyethylene glycol having 2 to 50 ethylene oxide units.

Embodiment 44. The polymeric micellar nanoparticle of any one of embodiments 22 to 39, wherein L⁴-R⁴ is polyethylene glycol having 5 to 25 ethylene oxide units.

Embodiment 45. The polymeric micellar nanoparticle of any one of embodiments 22 to 44, wherein z5 is 1.

Embodiment 46. The polymeric micellar nanoparticle of any one of embodiments 19 to 45, wherein the polymeric micellar nanoparticle is free of gadolinium (Gd).

Embodiment 47. The polymeric micellar nanoparticle of any one of embodiments 19 to 46, wherein the polymeric micellar nanoparticle is prepared from amphiphilic block copolymers.

Embodiment 48. The polymeric micellar nanoparticle of embodiment 47, wherein the amphiphilic block copolymer is an amphiphilic tri-block copolymer.

Embodiment 49. The polymeric micellar nanoparticle of any one of embodiments 19 to 48, wherein the polymeric micellar nanoparticle is a spherical polymeric micellar nanoparticle.

Embodiment 50. The polymeric micellar nanoparticle of any one of embodiments 19 to 48, wherein the polymeric micellar nanoparticle is a cylindrical polymeric micellar nanoparticle.

Embodiment 51. The polymeric micellar nanoparticle of any one of embodiments 19 to 50, wherein the polymeric micellar nanoparticle has a diameter of about 10 nm to about 1000 nm.

Embodiment 52. The polymeric micellar nanoparticle of any one of embodiments 19 to 51, wherein the polymeric micellar nanoparticle has a relaxivity value (r₁) of about 7×10⁻³ M⁻¹ S⁻¹ to about 10×10⁻³ M⁻¹ S⁻¹.

Embodiment 53. A method of providing visibility of an internal body structure of a subject undergoing MRI, the method including administering to the subject an effective amount of the polymeric micellar nanoparticle of any one of embodiments 19 to 52.

Embodiment 54. The method of embodiment 53, further including administering an additional contrast agent to the subject.

Embodiment 55. A method of detecting an internal body structure of a subject, the method including administering to the subject an effective amount of the polymeric micellar nanoparticle of any one of embodiments 19 to 52 and detecting the polymeric micellar nanoparticle using magnetic resonance imaging thereby detecting the internal body structure.

Embodiment 56. The method of embodiment 55, further including administering an additional contrast agent to the subject.

Embodiment 57. A method for synthesizing a polymeric micellar nanoparticle, the method including functionalizing a block copolymer amphiphile to incorporate a catechol group in one block of the block copolymer amphiphile, thereby providing a functionalized block copolymer amphiphile; contacting the functionalized block copolymer amphiphile under conditions suitable to afford a catechol-containing block copolymer; and contacting the catechol-containing block copolymer with a metal salt, thereby providing the polymeric micellar nanoparticle.

Embodiment 58. The method of embodiment 57, wherein the metal is iron.

Embodiment 59. The method of embodiment 57, wherein the block copolymer amphiphile is a tri-block copolymer amphiphile.

Embodiment 60. A diagnostic pharmaceutical composition including the polymeric micellar nanoparticle of any one of embodiments 19 to 52 and a pharmaceutically acceptable excipient.

Embodiment 61. A kit including an instruction and the polymeric micellar nanoparticle of any one of embodiments 19 to 52.

### VI. EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1

Magnetic resonance imaging (MRI) is a frequently used radiological imaging modality that has become increasingly important in the diagnosis of human disease. It is noninvasive and versatile, does not use ionizing radiation, and can be acquired at high resolution for obtaining anatomical and functional information on soft tissues. However, the low sensitivity inherent to MRI has led to the development of MRI contrast agents that increase sensitivity by catalytically shortening the transverse (T₁) and longitudinal (T₂) relaxation times of water protons. It is clear that, although effective, Gd-agents suffer from toxicity spurring a resurgence of interest in Gd-free MRI agents. For example, it has been reported that the administration of Gd-based contrast agents to patients with renal dysfunction may induce the severe disease, nephrogenic systemic fibrosis. Towards this goal, we have demonstrated here nanoscopic materials incorporating multiple Fe^{III}-based chelates as T₁-weighted MRI contrast agents with high efficiency and low toxicity.

While synthetic catechol-based polymers with Fe(III) have been widely described for a range of applications [13,17-23], limited examples have been explored as T₁-weighted MRI contrast agents. Indeed, the few systems that have been studied are synthetic melanin-based materials in the form of polydopamines generated by oxidative polymerization [12].

Current strategies for the construction of poly(Fe(III)-catecholate) as efficient T₁-weighted imaging agents for MRI, generally focus on the complexation of natural sepia melanin colloidal particles with Fe(III) salts [12,24,25]. However, the development of functional and robust contrast agents from melanin-type materials has met with profound challenges, which are yet to be resolved. Issues include limited control over the synthetic colloidal chemistry hindering size and shape control over the resulting melanin particles [17]. Without wishing to be bound by any theory, we believe that more sophisticated strategies making use of self-assembled soft materials from amphiphilic block copolymers [26-30], with the integration of Fe(III)-catecholate blocks, may provide an avenue for preparing particles of well-defined and predictable morphologies. Certainly, these are undoubtedly critical, and highly desirable properties if one aims to prepare materials for in vivo use. This would be enabled by the use of a controlled living polymerization method, giving rise to well-defined and reproducibly accessible block copolymer architectures. The resulting supramolecular nanostructures with rigorously controlled physical parameters (i.e. size, shape and composition) represent a new class of macromolecular Gd-free MRI contrast agent. Control over these parameters is a must as it is widely known that the fundamental physical properties of nanoparticles may affect their behavior within biological systems [31,32]. We report herein effort towards this goal through the design and synthesis of catechol-based amphiphilic block copolymers and the elucidation of the relaxation properties of the resulting self-assembled micellar nanoparticles.

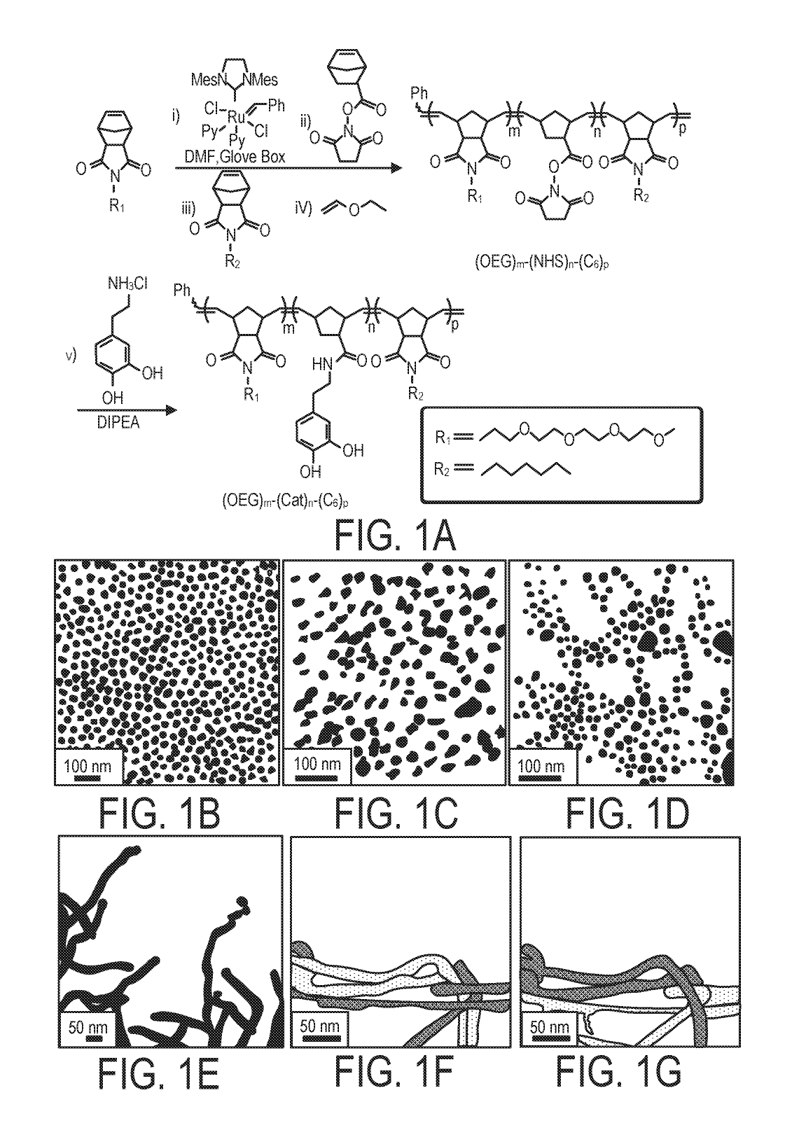

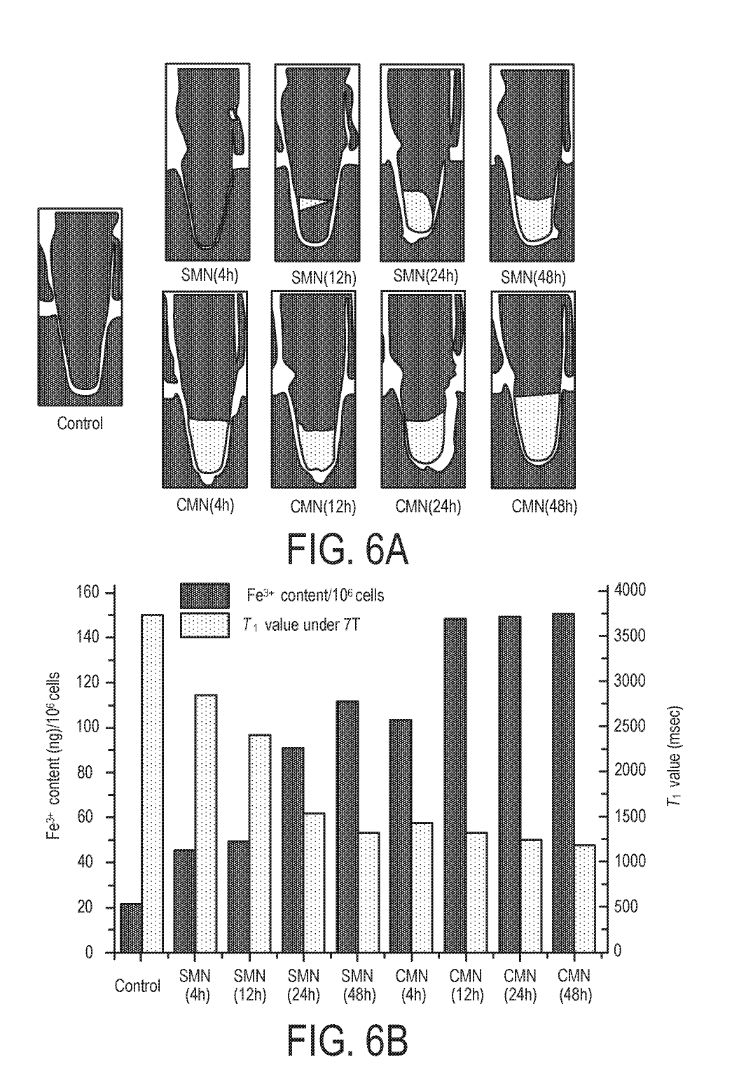

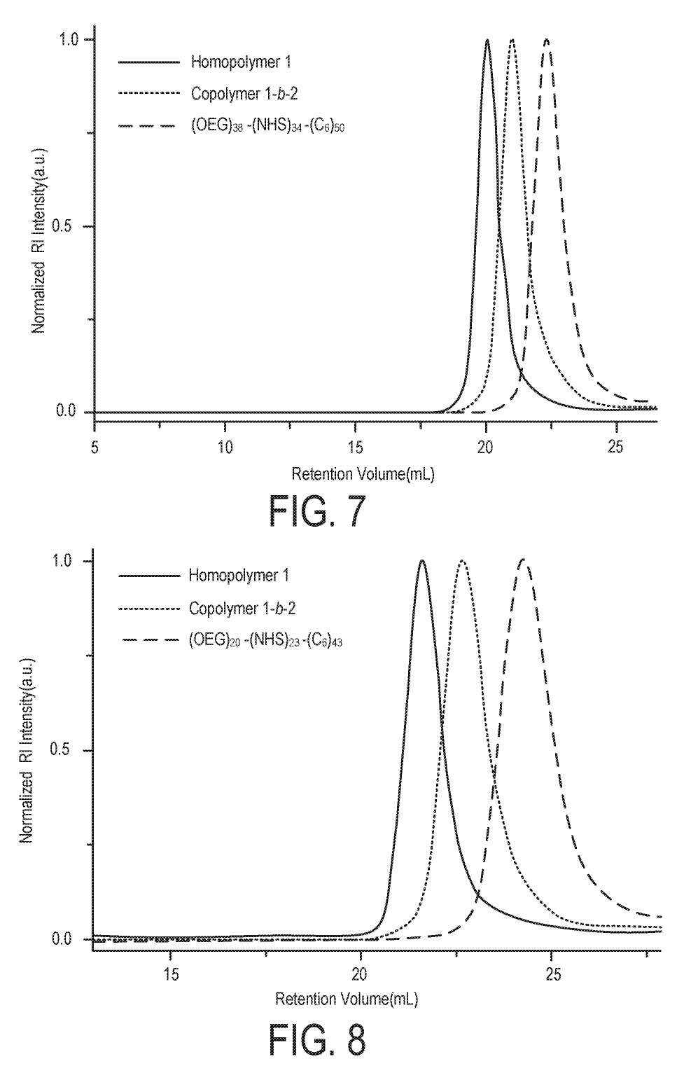

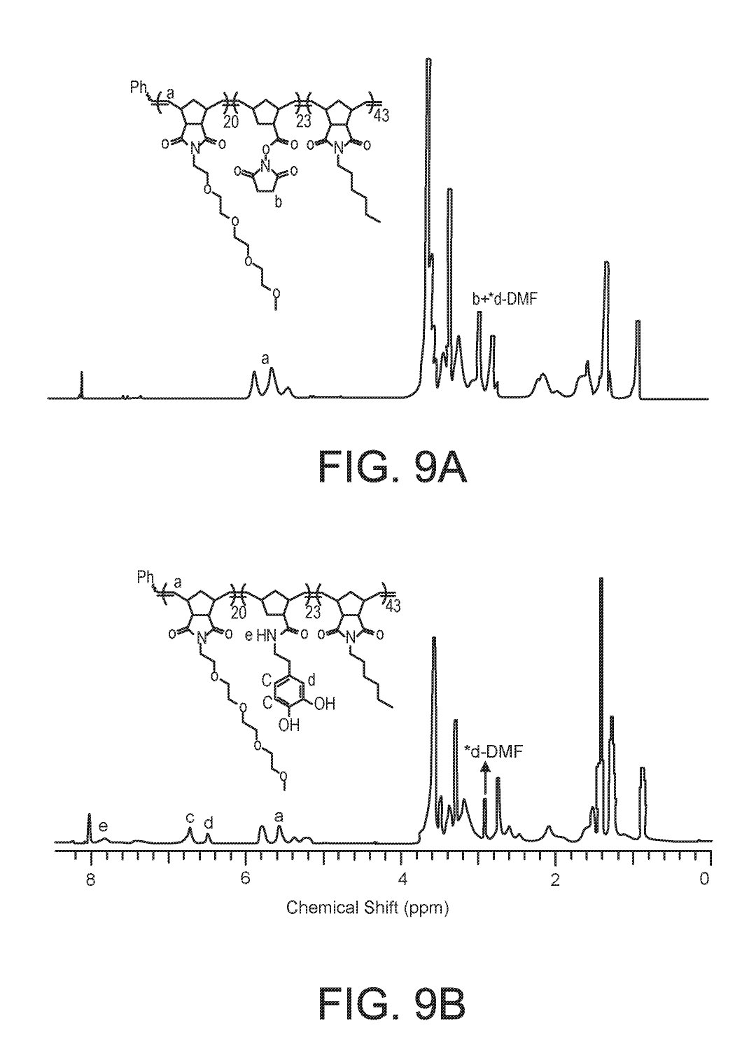

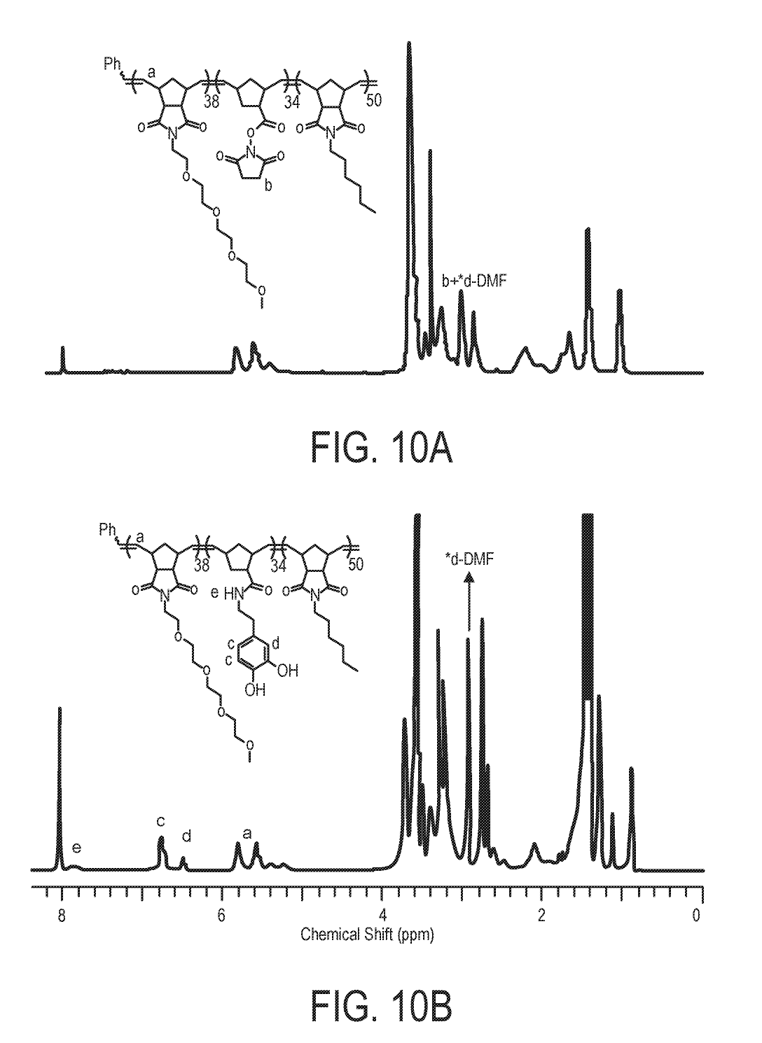

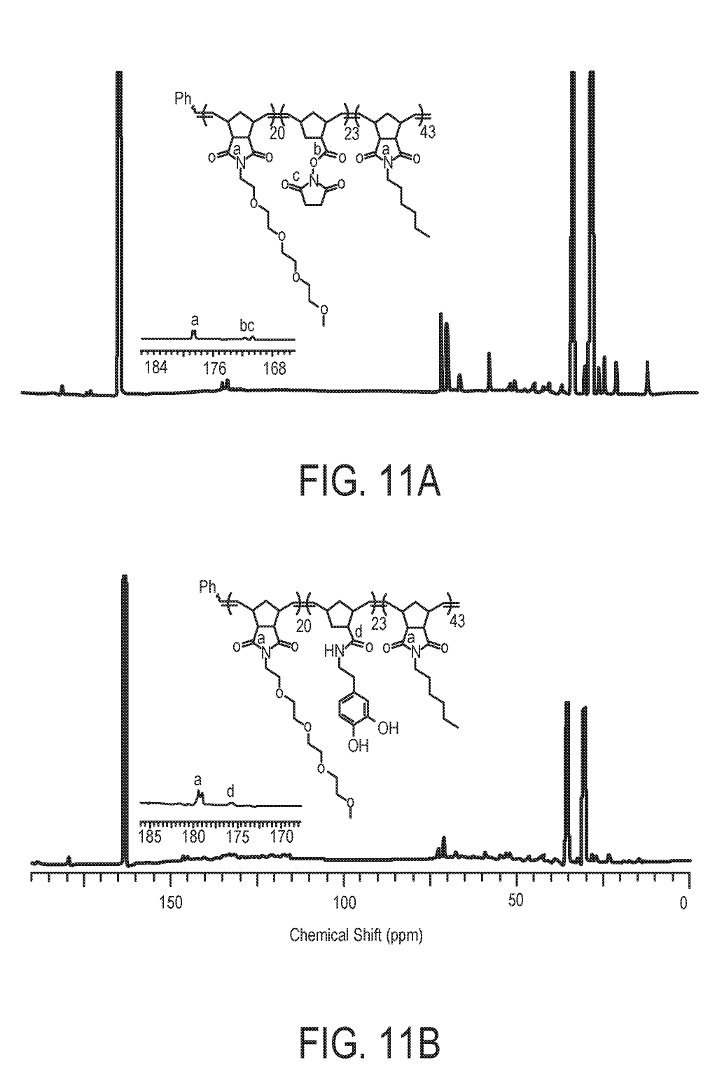

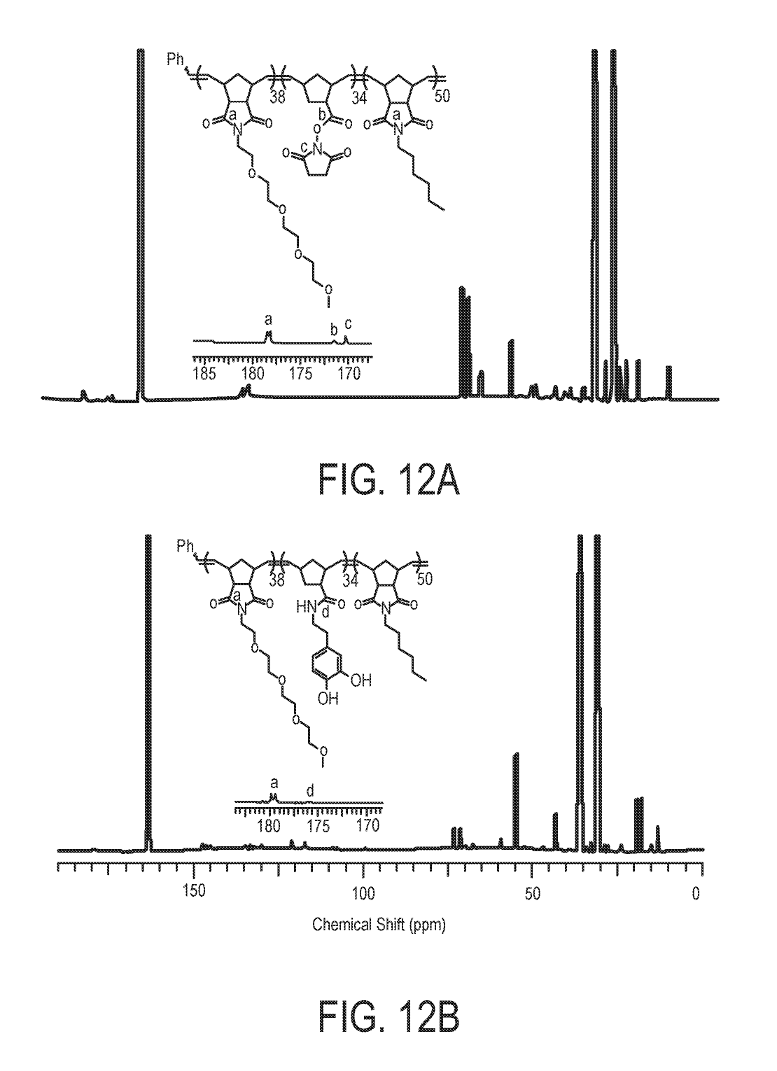

Molecular Design and Micellar Nanoparticle Formation. Our synthetic approach employed post-polymerization functionalization [28,29] for the incorporation of multiple catechol groups localized in the middle block of a tri-block copolymer amphiphile (FIG. 1A). The macromolecular precursors, (OEG)ₘ-(NHS)ₙ-(C₆)ₚ, were directly synthesized via ring-opening metathesis polymerization (ROMP) [33] (FIGS. 7-8) using a modified 2nd generation Grubbs' catalyst (FIG. 1A). Excess dopamine hydrochloride was then added to the macromolecular precursors in the presence of N,N-Diisopropylethylamine (DIPEA) to afford the final products. Notably, a post-polymerization functionalization route was taken when it was discovered that catechol-modified norbornene monomers would not polymerize utilizing this class of initiators for ROMP [21]. Regardless, this synthetic approach achieved a near quantitative incorporation of catechol groups into the middle region of the block copolymers as determined by the ¹H NMR and ¹³C NMR (FIGS. 9A-9B, 10A-10B, 11A-11B, 12A-12B). Both of the resulting amphiphilic tri-block copolymers (Polymers 1 and 2) are stiff solids with limited solubility in nonpolar organic solvents. By varying the segment size of each block in the amphiphilic copolymers to tune the volume fraction of hydrophobic domain, two kinds of stable micellar morphologies (i.e. sphere and cylinder) could be obtained (FIGS. 1B-1G).

The assembly of the resulting catechol-based amphiphilic block copolymers was performed in a selective solvent to generate the two different micelles [34]. Specifically, to prepare a spherical micellar nanoparticle (SMN), an aqueous solution of FeCl₃ (1 mg/mL) was added at a rate of 10 μL/hour to a vial containing 2 g of a stock solution of Polymer 1 in THF as a common solvent, with an initial concentration of 2.0 wt % until the final water content reached 70 wt %. The stable SMN were then obtained by dialyzing the micelle solution against deionized water for 3 days to remove the organic solvent and any unchelated Fe(III). Cylindrical micellar nanoparticles (CMN) were generated using Polymer 2 which consists of a higher volume fraction of the hydrophobic domain, under precisely the same condition [35].

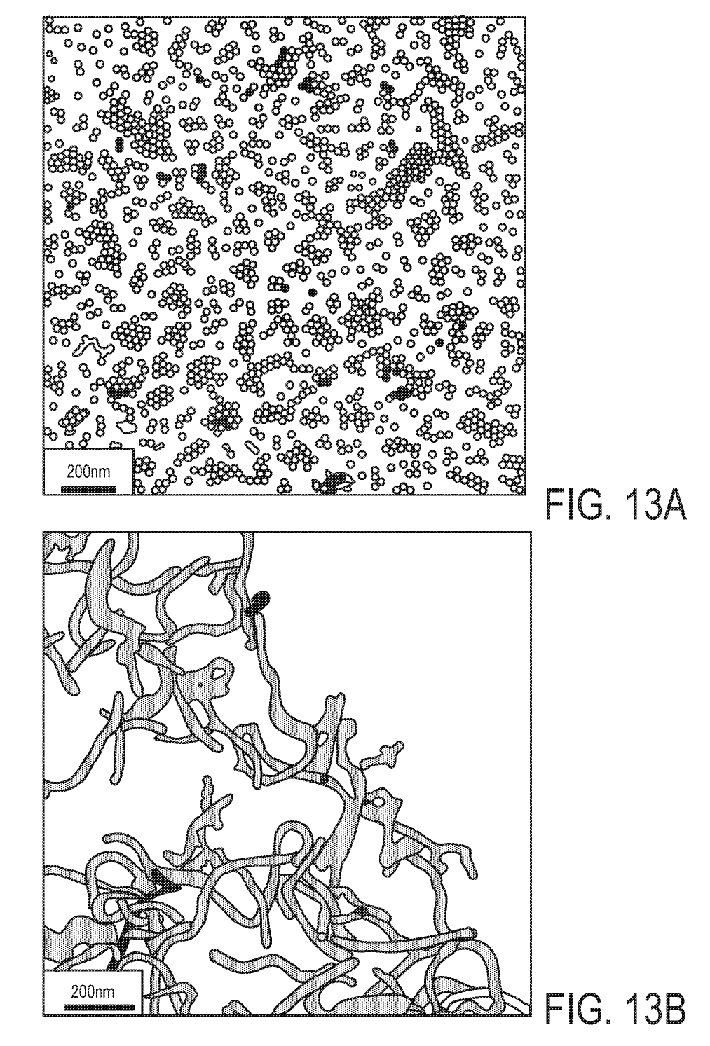

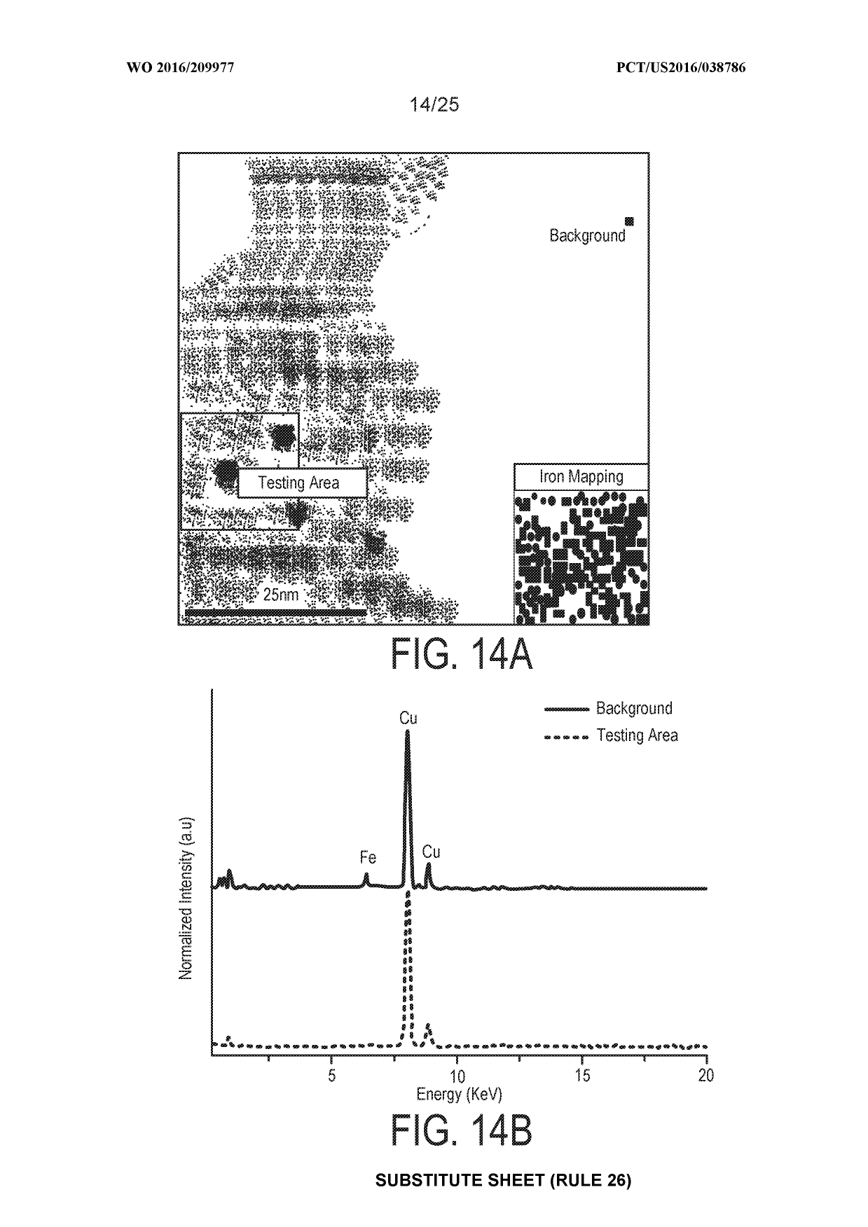

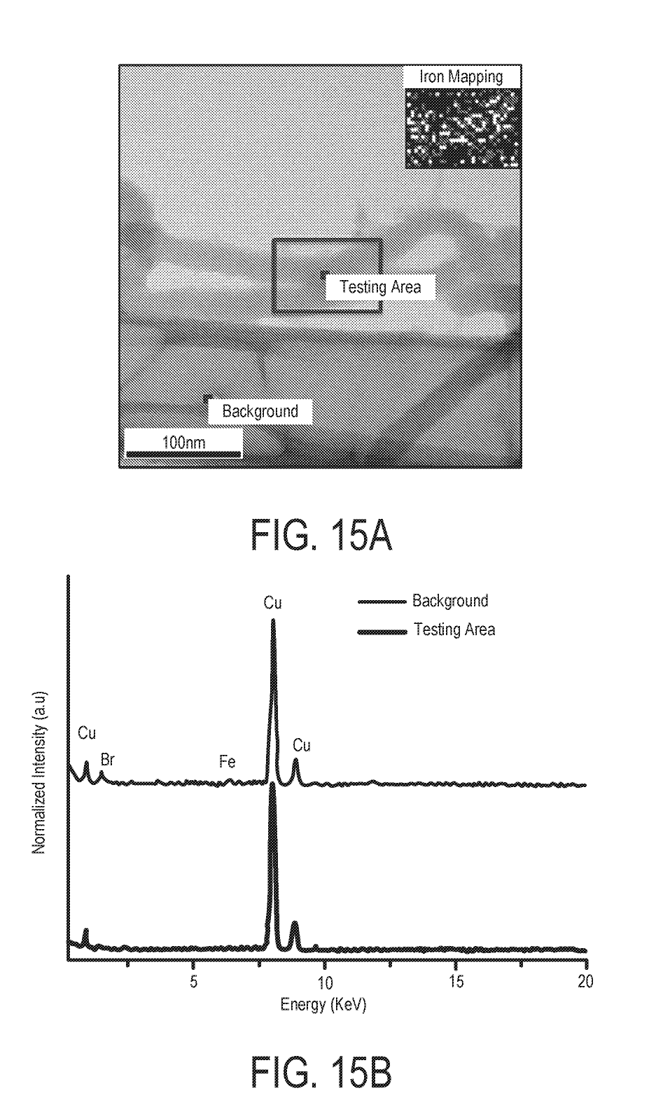

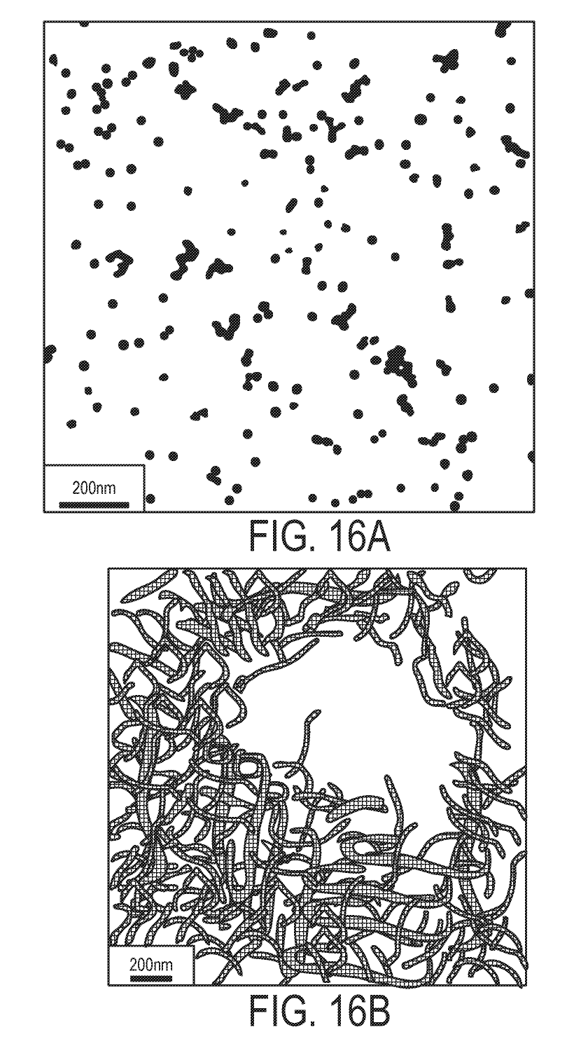

Each of the two well-defined micellar morphologies were characterized by cryo-transmission electron microscopy (TEM) (FIGS. 1B and 1E) and dry state TEM (FIGS. 13A-13B), demonstrating diameters for SMN and CMN of approximately 30 nm and 25 nm, respectively. The presence of high contrast metal elements (heavy nuclei) in both SMN and CMN were evident in bright field scanning transmission electron microscopy (BF-STEM) (FIGS. 1E and 1F) and high angle annular dark field (HAADF)-STEM (FIGS. 1D and 1G). Moreover, selected area BF-STEM coupled with energy dispersive X-ray spectroscopy (EDS) confirmed the presence of Fe(III) ions localized in the micellar nanoparticles (FIGS. 14A-14B, 15A-15B). Specifically, the EDS profiles suggested that the content of iron in the testing areas of SMN and CMN were significantly higher than those on the grid surface background, which are in good agreement with the elemental mapping analysis results (FIGS. 14A and 15A). Furthermore, TEM was used to confirm that both SMN and CMN are stable in aqueous solution for at least 6 months (FIG. 16A-16B).

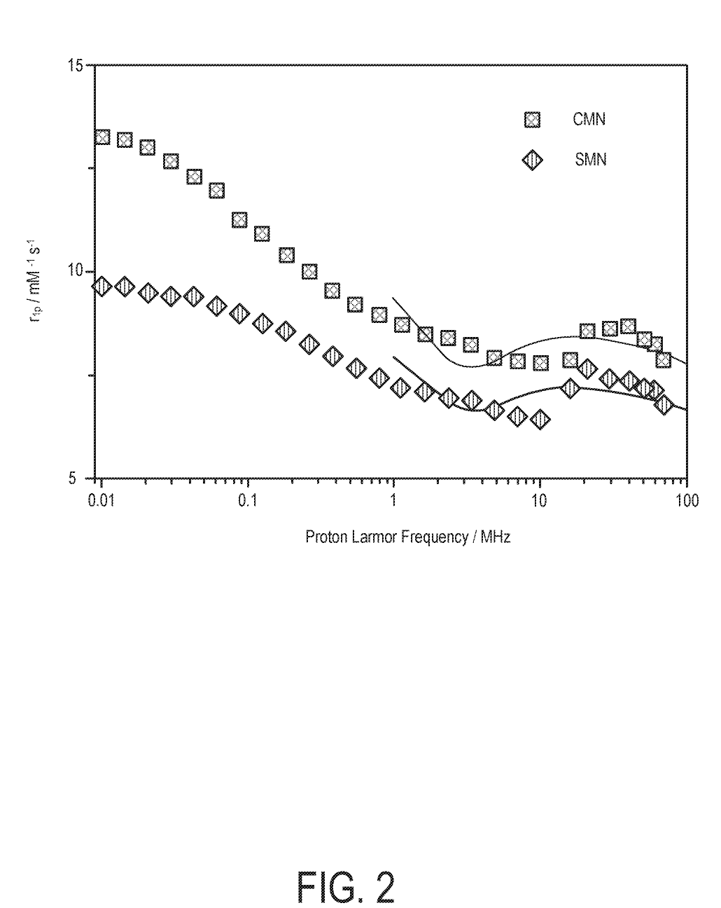

Relaxometric Characterization of Micellar Nanoparticles. The basic relaxation properties of SMN and CMN were first investigated using their ¹H 1/T₁ nuclear magnetic relaxation dispersion (NMRD) profiles acquired under magnetic field strengths from 0.01 MHz to 70 MHz (FIG. 2). Inductively coupled plasma-optical emission spectrometry (ICP-OES) was employed to calibrate the Fe(III) concentration of SMN and CMN solutions. The NMRD profiles show a similar shape and different amplitude. In both cases there is a poorly defined plateau at low fields (ca. 0.01-0.05 MHz), followed by a wide dispersion (approx 0.05-7 MHz) and by a broad hump at higher frequencies. The ratio of the relaxivity values at low (0.01 MHz) and high fields (60 MHz) amounts to 1.6 and 1.4 for CMN and SMN, respectively. Relaxivity, r₁ₚ, arises from metal-bound and/or proximate hydrogen-bonded water molecules, dipolarly interacting with the unpaired electrons of the metal ion:

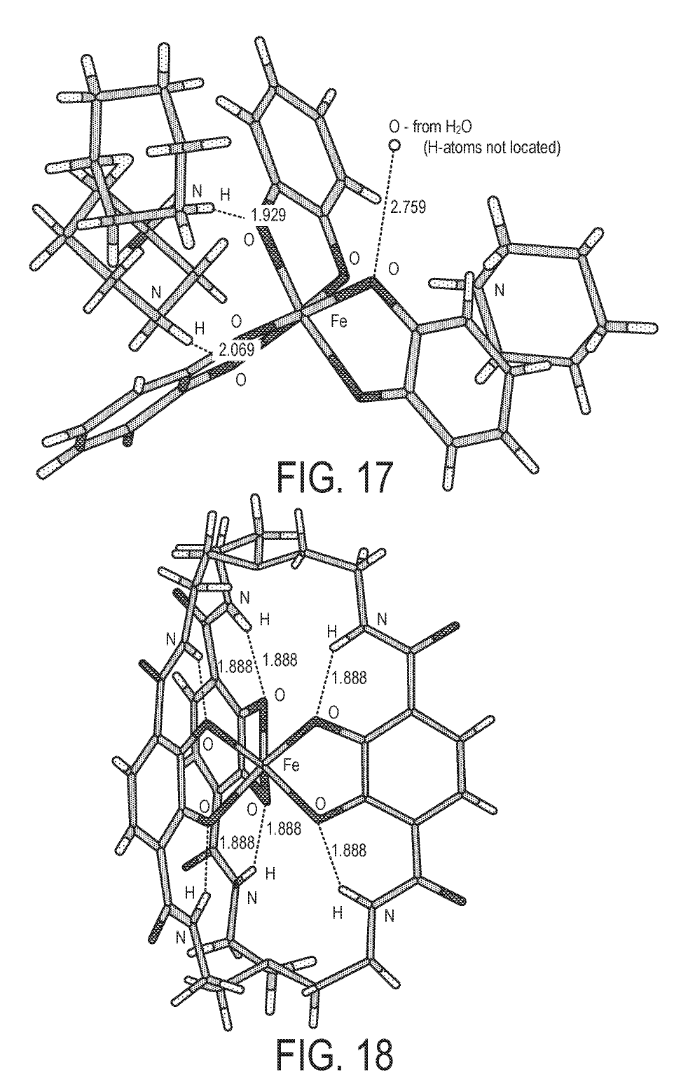

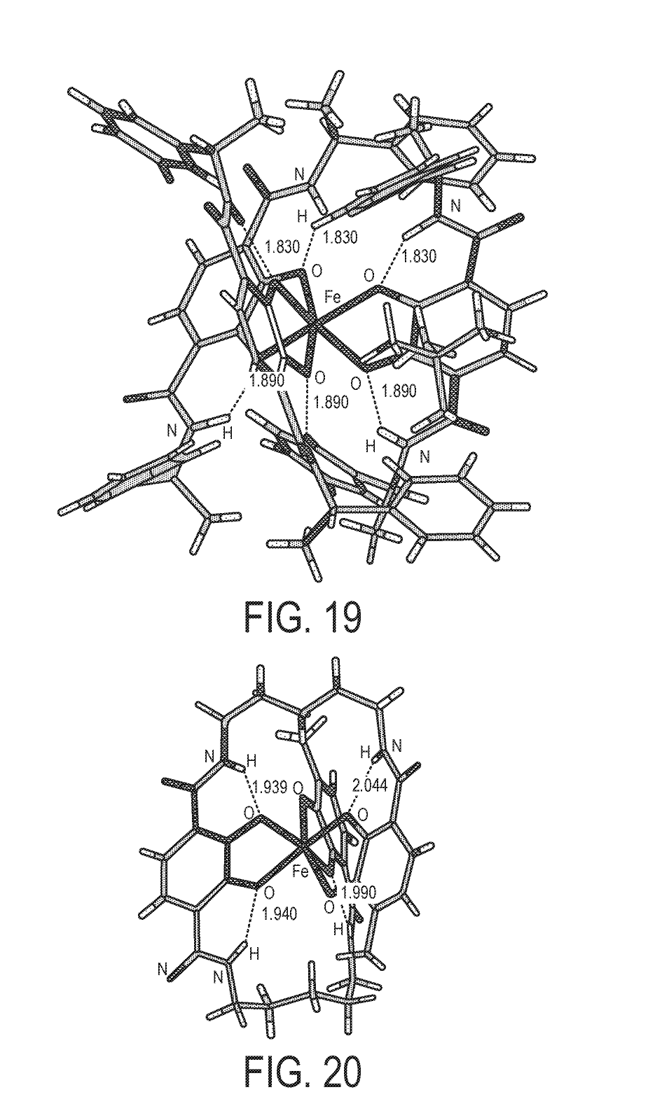

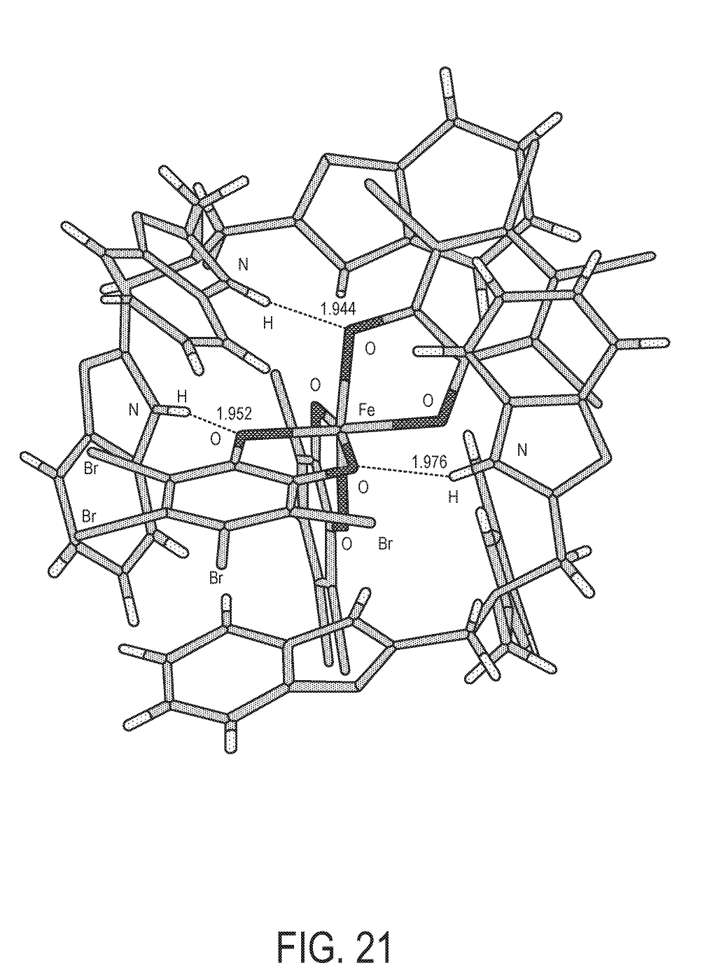

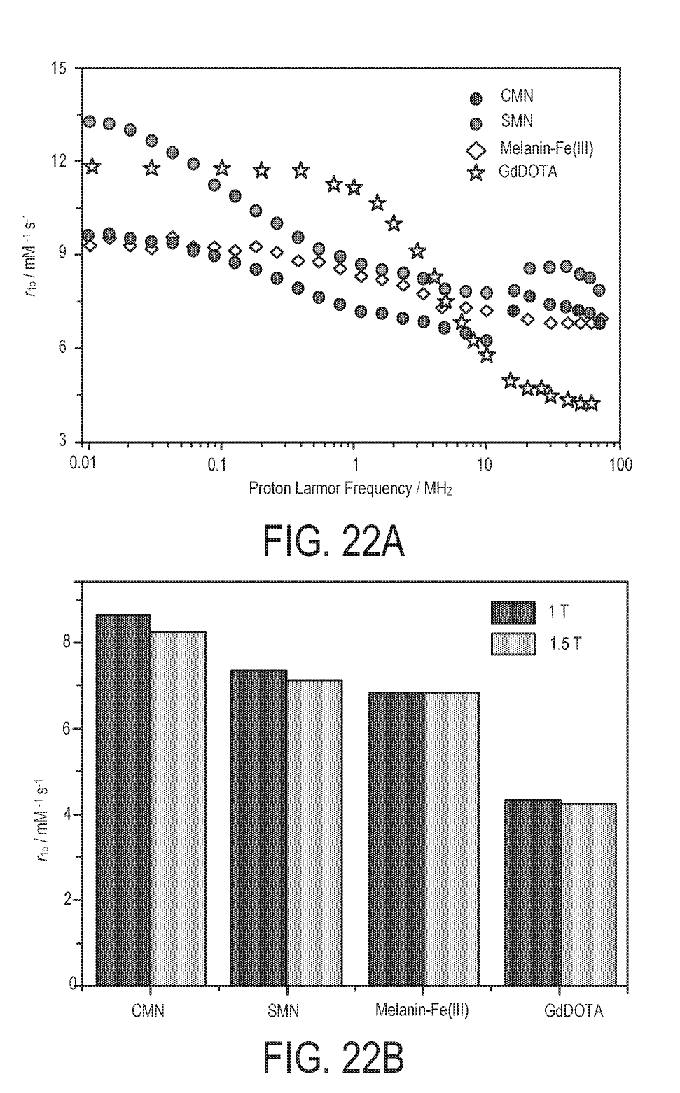

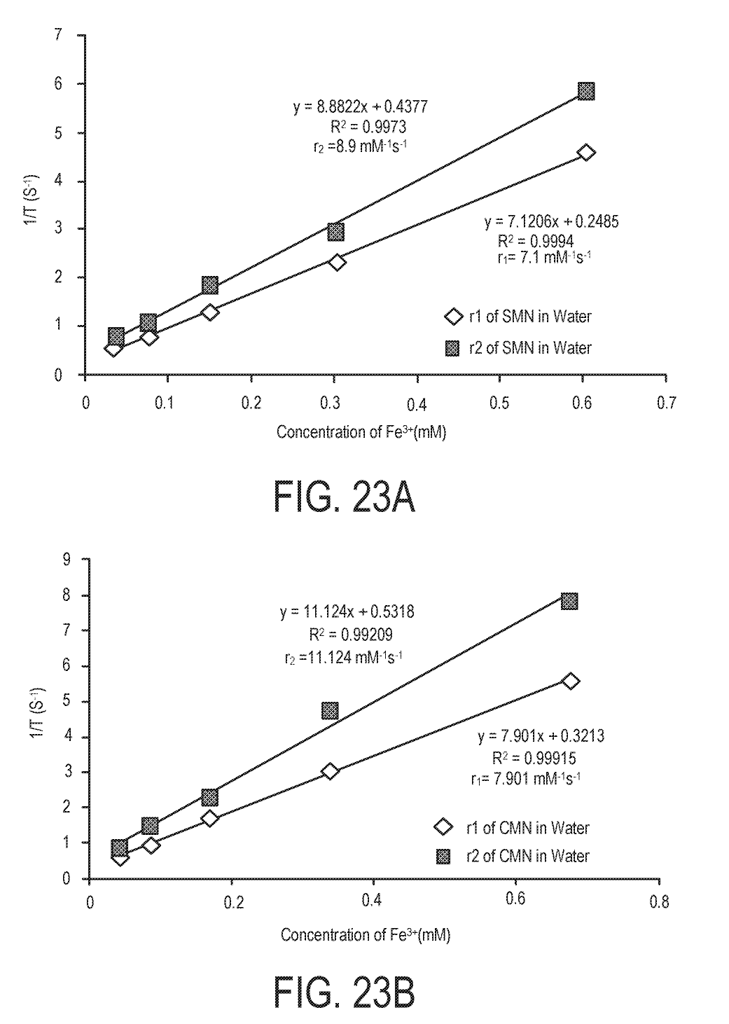

r₁=f_{M}×(T_{1M}+τ_{M})⁻¹+r₁^{os} (1)

where f_{M} is the mole fraction of interacting water molecules, T_{1M} their proton relaxation time due to the paramagnetic Fe(III) ion, τ_{M} the exchange lifetime and r₁^{os} the contribution of outer-sphere fast diffusing water protons [6-8]. T_{1M} depends on r⁶, the distance of the interacting dipoles, on the correlation time (τ_{C}) for the proton-electron dipolar interaction and on ω_{I} and ω_{S}, the proton and electron Larmor frequencies, respectively. The inflection point in the profiles reflects the condition ω_{S}τ_{C}=1, so that we can estimate for the correlation time a value of about 0.4-0.6 ns. The appearance in both profiles of broad humps at high fields with relaxivity values well above 6 mM⁻¹s⁻¹ represent a strong indication that the relaxivity is not dominated by a simple outer-sphere mechanism. Considering that the direct binding of an inner sphere water molecule to a coordinatively saturated, six-coordinate, Fe^{III} (catecholate)₃ site is unlikely, we suggest the enhanced relaxivity originates from presence of one more second-sphere interactions [36-38], like many established natural or synthetic melanin-Fe³⁺ systems [12]. These second-sphere interactions likely take the form of dynamic hydrogen bonds between H₂O and polar groups on the nanoparticle proximal to Fe^{III}(catecholate)₃ sites, or to oxygen atoms of the Fe^{III}(catecholate)₃ sites themselves. Indeed, several structurally characterized Fe^{III}(catecholate)₃ complexes have been reported that feature hydrogen bonds between oxygen atoms of the FeO₆ core and polar H—X units (X=O, N) (see five examples from Cambridge Structural Database listed in FIGS. 17-21). Moreover, direct X-Ray crystallographic evidence for hydrogen bonding between the hydrogen atom of water and the oxygen atom a transition-metal catecholate has been observed in both Ni [39] and Mn [40] complexes, thereby probably suggesting that such second-sphere H₂O interaction modes are viable in these nanoparticle systems.

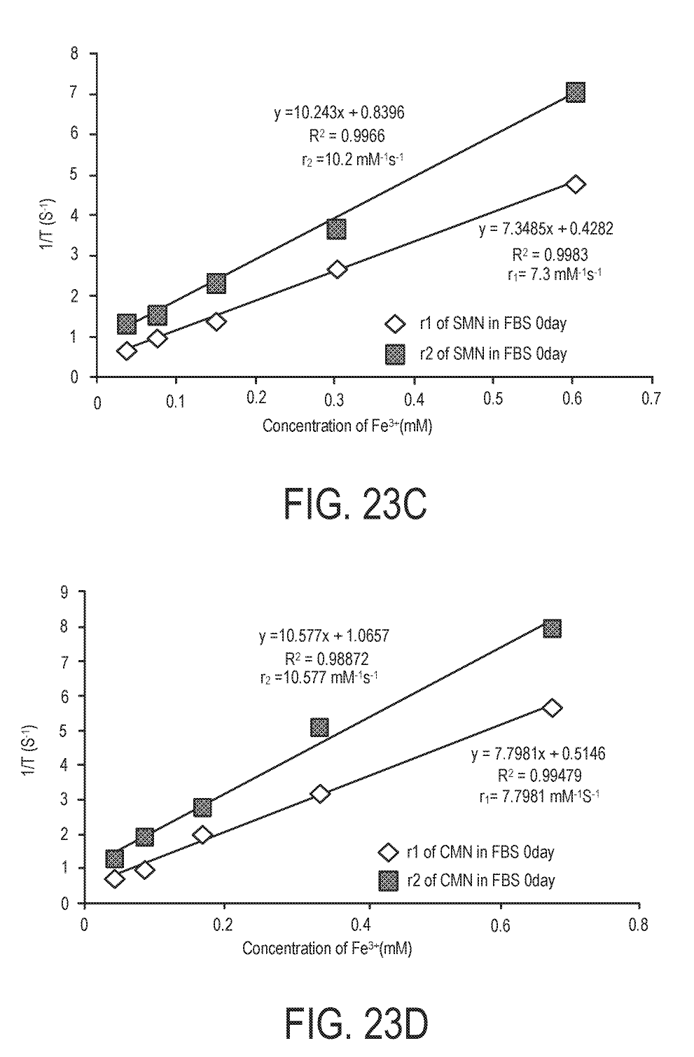

Therefore, based on this precedent, we could fit the data to calculate rotational correlation times of 490 and 448 ps for CMN and SMN respectively, assuming the presence of two second-sphere water molecules at a long-range distance of 3.3±0.1 A from the Fe center with a residence lifetime of ca. 2 μs [41]. These results are in good agreement with similar findings reported for bovine lactoferrin [41] and methemoglobin [42]. In addition, both SMN and CMN exhibit substantially higher r₁ than that of mononuclear Fe(III)-catecholate complexes (i.e. at a field of 20 MHz, r₁ values for SMN and CMN are 8.0 mM⁻¹s⁻¹ and 9.0 mM⁻¹s⁻¹, respectively, while r₁ for small molecular Fe-catecholate complexes are only about 2.0 mM⁻¹s⁻¹) [15] indicating that the macromolecular scaffolds effectively increase the relaxivity of contrast agent moieties by restricting the rotational mobility of the complex (i.e. of the vector connecting Fe(III) and the protons of second sphere water molecules) [43,44]. Moreover, the per Fe^{III} r₁ₚ of SMN and CMN outperforms clinically used Gd^{III} contrast agents and synthetic melanin-Fe(III) complex [12] across a wide range of applied magnetic field strengths (>10 MHz). For example, measured enhancements over GdDOTA are +100% and +69% for CMN and SMN, respectively, at 1.0 T and 298 K (FIG. 22A-22B).

It should be noted that NMRD profiles are fitted herein only in the high field region because of the known limitations of Solomon-Bloembergen-Morgan (SBM) theory in the slowly rotating regime that render it unable to completely account for the behavior of slowly rotating systems at very low magnetic field strengths, where the Zeeman energy is smaller than the zero field splitting energy [45]. In addition, it is worth mentioning that the deviation of the profiles from the behavior of a well-defined Lorentzian might be compatible with the presence of different non-equivalent Fe(III) ions in the nanoparticles [46]. The NMRD profiles may reflect a different structure and dynamics (number, distance and lifetime) of second sphere water molecules around each Fe centers, which have characteristic and different electronic relaxation times.

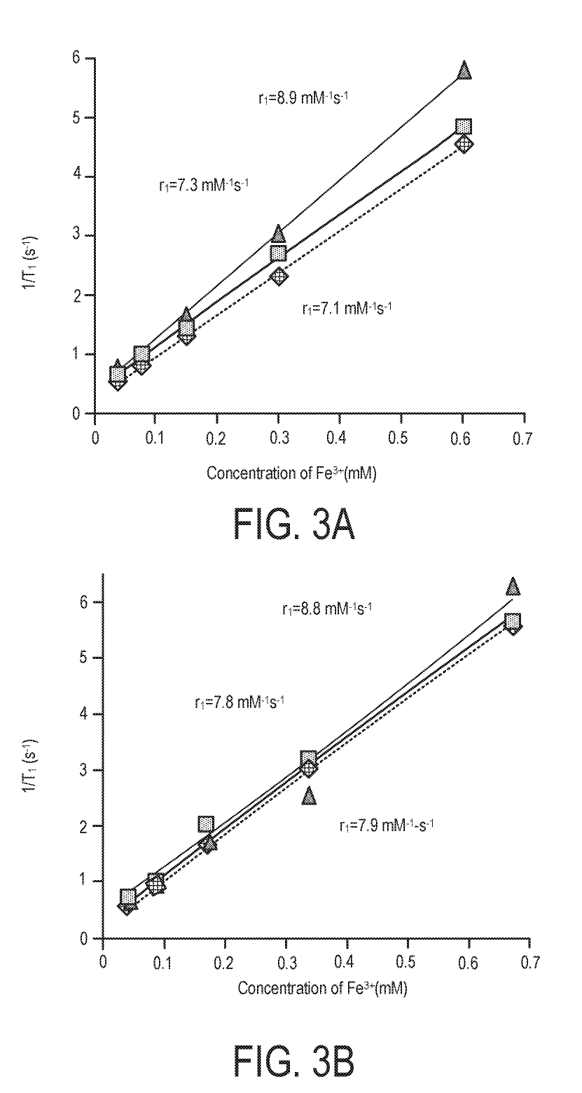

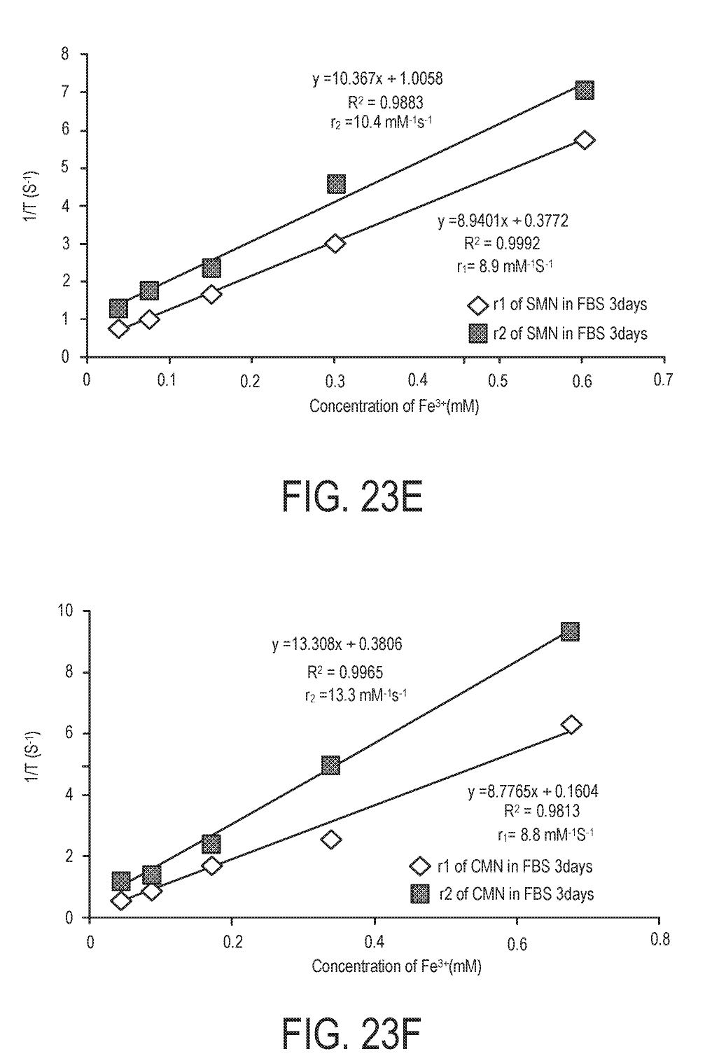

The longitudinal and transverse relaxation times (T₁ and T₂) of both nanoparticles, with various concentrations, at clinically relevant field strengths (Bₒ=1.41 T) were measured using time-domain NMR to quantitatively calculate their relaxivity values (r₁ and r₂) (FIGS. 3A-3B and FIGS. 23A-23F). Both SMN and CMN exhibit high relaxivity values (r_{1,SMN}=7.1 mM⁻¹s⁻¹, r_{1,CMN}=7.9 mM⁻¹s⁻¹) for potential clinical usage (based on the calculated results shown in FIGS. 3A, 3B, 22A, 22B and Table 1-1) [12]. In summary, the excellent contrast enhancement of the Fe(III)-chelated micellar nanoparticles was mainly due to second-sphere contributions, as discussed above. Moreover, the low r₂/r₁ ratios of both SMN and CMN (r₂/r₁=1.25 for SMN, and r₂/r₁=1.40 for CMN) favor positive contrast enhancement (brightening) because the interference from T₂ effects (darkening) are relatively small. Overall, the high T₁ relaxivity and low r₂/r₁ ratio of both micellar nanoparticles could make them suitable as Gd-free clinical contrast agents for T₁-weighted MRI. This conclusion is supported by the bright T₁-weighted MR images from SMN and CMN in aqueous solution (FIG. 3C).

TABLE 1-1
Selected relaxation parameters obtained from the analysis of NMRD profiles reported
in FIG. 2.*

| | ²⁰r₁ₚ | Δ² | τ_{V} | τ_{R} | r | | τ_{M} |
|---|---|---|---|---|---|---|---|
| Micelles | (mM⁻¹ s⁻¹)^{a} | (10¹⁹ s⁻²)^{b} | (ps)^{b} | (ps) | (Å) | q^{c} | (μs)^{b} |
| SMN | 7.7 | 1.4 ± 0.1 | 54 ± 3 | 448 ± 8 | 3.3 ± 0.1 | 2 | 2.0 ± 0.1 |
| CMN | 8.6 | 1.1 ± 0.2 | 50 ± 4 | 490 ± 12 | 3.3 ± 0.1 | 2 | 1.9 ± 0.2 |

*^{a} 20 MHz and 298 K; ^{b}the parameters for electronic relaxation are used as empirical fitting parameters and do not have a real physical meaning for slowly tumbling nanosized systems. Low-field data, those most affected by electronic relaxation, were not included in data analysis; ^{c}fixed during the fit.

TABLE 1-2
Relaxivity Data for Micellar Nanoparticles in Different Media.*

| Micelles | Media | r₁ (mM⁻¹s⁻¹) | r₂ (mM⁻¹s⁻¹) | r₂/r₁ |
|---|---|---|---|---|
| SMN | H₂O | 7.1 | 8.9 | 1.25 |
| CMN | H₂O | 7.9 | 11.1 | 1.40 |
| SMN | FBS (0 day) | 7.3 | 10.2 | 1.40 |
| CMN | FBS (0 day) | 7.8 | 10.6 | 1.36 |
| SMN | FBS (3 days) | 8.9 | 10.4 | 1.17 |
| CMN | FBS (3 days) | 8.8 | 13.3 | 1.51 |

*The magnetic field strength for T₁ and T₂ measurement is 1.41 T.

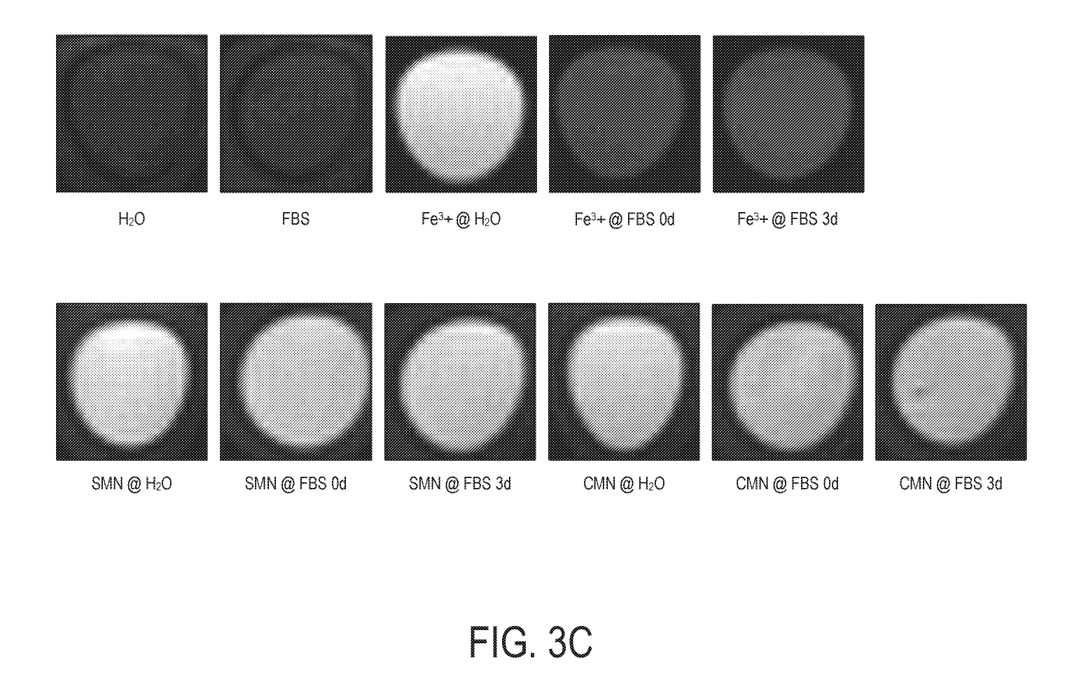

Variable field and temperature magnetic measurements were performed by using a superconducting quantum interference device (SQUID) to confirm that the observed MRI contrast arises from multiple isolated Fe(III)-catecholate sites. Plots of magnetization versus applied magnetic field show significant curvature only at temperatures less than 24 K, consistent with isolated paramagnetic iron centers (FIGS. 4A-4B). The lack of magnetic saturation, even at 7 T and 2 K, indicates a significant magnetic anisotropy associated with the spin state and coordination environment of the Fe(III); however, quantitative fitting of the data was not possible. This indicates that a range of Fe(III) environments may be present within SMN and CMN micelles, as suggested by the broad NMRD profiles. At temperatures exceeding 24 K, the magnetization is completely linear with applied field, confirming that the MRI contrast ability of SMN and CMN results from many isolated Fe(III)-catecholate complexes inside the micellar nanoparticles.

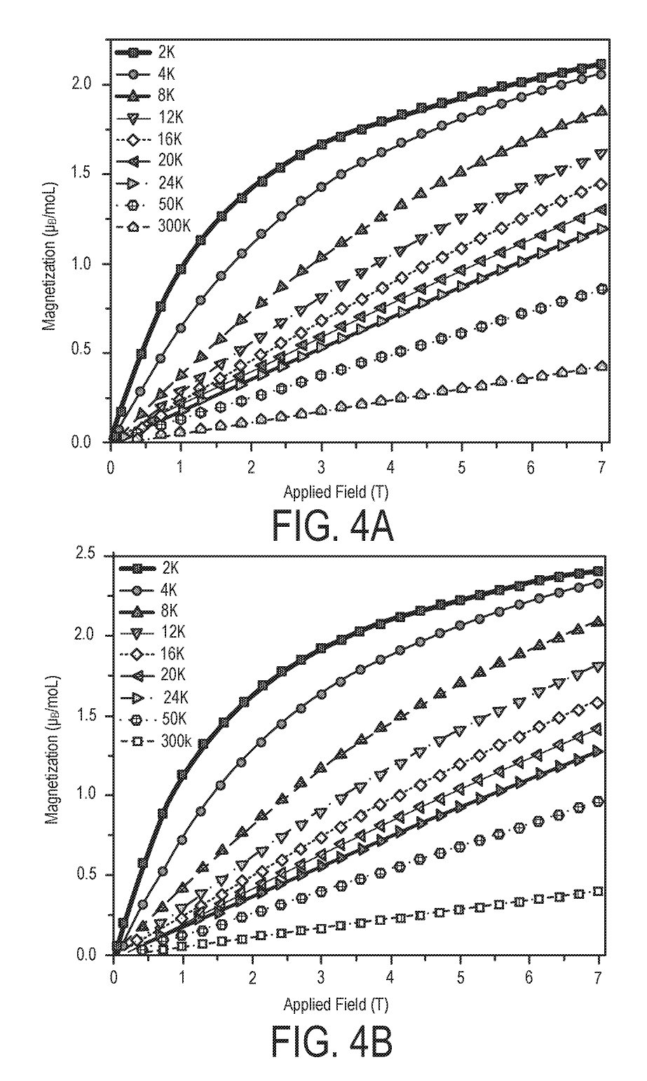

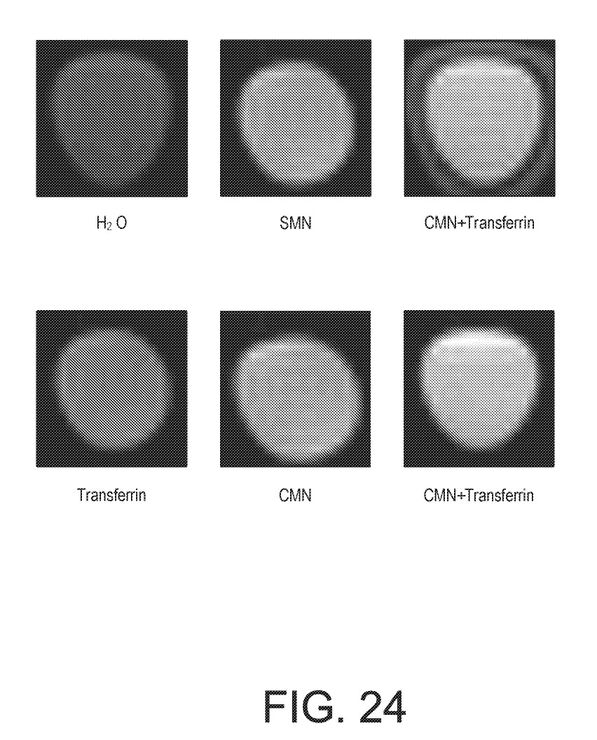

Stability and Cytotoxicity of Micellar Nanoparticles. The stability of SMN and CMN in serum was also addressed by examining them in two respects over time: 1) No collapse, or change of micellar nanoparticle morphologies were observed; and 2) The total metal ion content and relaxivity values of Fe(III)-chelated nanoparticles does not change significantly. With regards to morphological stability, without wishing to be bound by any theory, it is believed that although many previously established micelle-based therapeutic systems have suffered from an inherent instability in vivo (generally undergoing collapse or break-up in the presence of serum lipids and proteins [47]), the micellar particles described herein benefit from cross-linking networks of individual polymer chains in the micellar interface via the formation of interchain, multiple Fe-catecholate coordination bonds [14,48-51]. These crosslinks lead to the particles being stabilized for more than 20 days in fetal bovine serum (FBS, 100%) even at a high concentration (>20 mg/mL). With regards to the strong binding capacity and affinity of catechols for Fe(III) [13], we performed a stability assay of Fe(III)-chelated SMN and CMN in PBS and FBS solution to show that there was no further ion release from micellar nanoparticles at different incubation time points (FIGS. 5A-5B). It could be noted that the metal ion contents of SMN and CMN in both of PBS and FBS solution are almost about 100% after 7 days. Accordingly, the r₁ and r₂ values of both SMN and CMN after 3 days incubation in FBS are almost identical to the ones corresponding to freshly-prepared micellar nanoparticles (FIGS. 3A, 3A, 23A-23F and Table 1-1), which is in good agreement with the their T₁-weighted MR images (FIG. 3C). It was found that the intensity of bright MR images for several SMN and CMN samples in different media with various incubation times is also very similar. However, the MRI signal from free Fe(III) was observed to be immediately quenched in FBS (FIG. 3C), likely due to the participation of free ions in redox reactions in the biological fluid [52]. This result also suggests that there were no further metal ions leaking from the nanoparticles after 3 days incubation in serum since the MR images do not lose intensity over that time scale (FIG. 3C). More interestingly, it was even observed that the brightness of MR images for both SMN and CMN samples after 24 hour incubation with excess transferrin protein (13 mg/mL) is still quite similar to the freshly-prepared samples (FIG. 24). Above all, we conclude that the evidence confirms the stability of this class of micellar nanoparticle-based MRI agents in biological media.

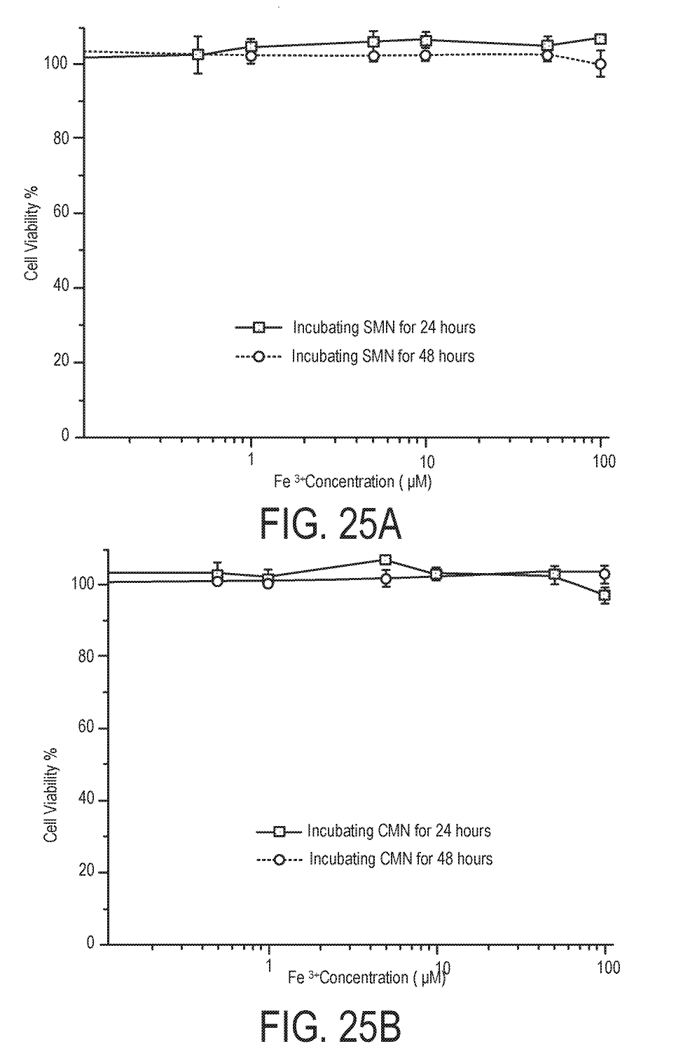

Shape-and Time-Dependent MR Imaging of Micellar Nanoparticles in Hela Cells. Prior to investigation of the MR imaging performance of prepared micellar Nanoparticles in Hela cells, their cytotoxicity at various concentrations of Fe(III) was assessed using an CCK based toxicology assay. Similar to Fe(III)-chelated melanin colloidal particles [12], both SMN and CMN show high biocompatibility and promising low toxicity with respect to live cells. Cell viability was measured in Hela cells using micellar nanoparticles with various dosages from 0.5 μM to 100 μM Fe(III) for 24 h- and 48 h-incubation. Under these conditions, the cell viability was maintained at around 100% in all groups (FIGS. 25A-25B).

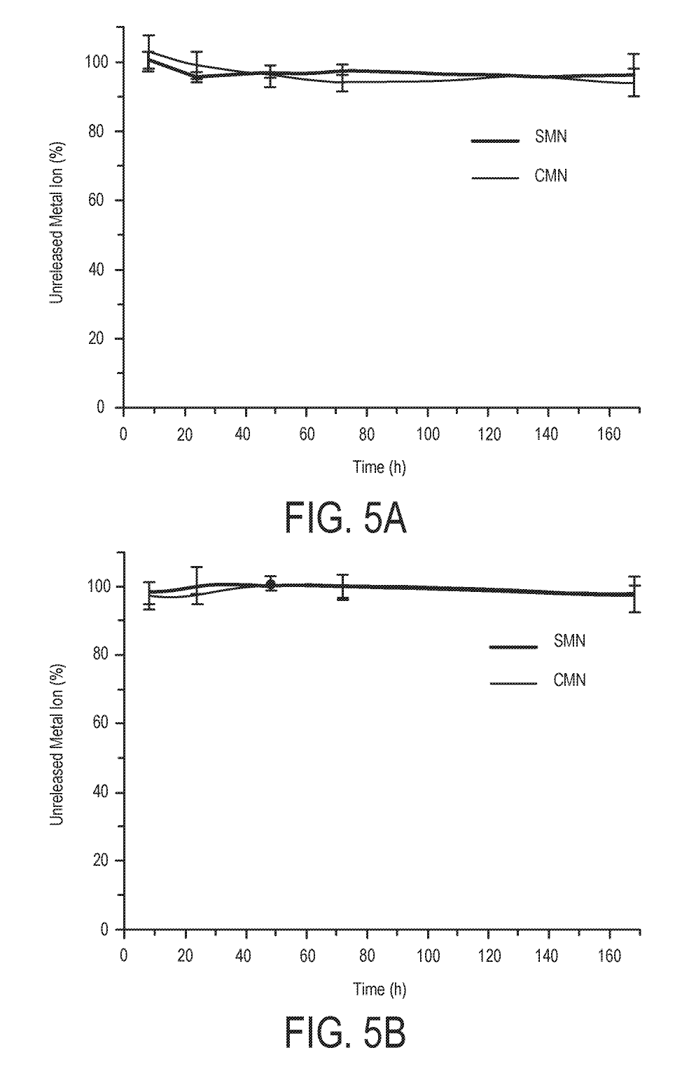

To further investigate the shape- and time-dependent in vitro MRI performance of those two micellar nanoparticles, SMN and CMN with identical [Fe³⁺] concentration (67.5 μM) were incubated with Hela cells for different periods of time, and the quantitative analysis of iron uptake and corresponded T₁ relaxation values was measured by ICP-OES and Bruker 7.0 T magnet, respectively. In general, Hela cells incubated with SMN and CMN both exhibited enhanced positive contrast in the T₁-weighted MR images compared to control cells (time incubation time 24 h or 48 h) (FIG. 6A). However, it was also found that T₁-weighted MR images of Hela cell pellets incubated with CMN exhibited much stronger T₁ signal enhancement (shorter T1 relaxation time) compared with those incubated with SMN after short incubation times (4 h or 12 h) (FIG. 6A). Quantitative analysis of intracellular iron indicated cell uptake of these two kinds of micellar nanoparticles was indeed shape- and time-dependent (FIG. 6B). For CMN, the uptake of nanoparticles significantly increased in the first 4 h, but the uptake rate gradually slowed and reached a plateau at 12 h, which is in good agreement with T₁ value of each corresponded cell pellet. Nevertheless, the intracellular Fe³⁺ content of SMN was gradually increasing without saturation after even 48 h (FIG. 6B), perfectly matching with the decreased T₁ values of cell pellets with the increasing incubation time. Interestingly, CMN-associated MR imaging of Hela cells usually exhibits brighter positive contrast and shorter relaxation time than the SMN-associated one with the identical initiate [Fe³⁺] concentration and incubation time, particularly when the short incubation time (i.e. 4 h) was employed.

In terms of mechanism, it is possible that these differences are explained by the fact that CMN is capable of making multiple contacts with the cell surface providing an initially stronger association leading to faster and more efficient uptake. Indeed, in this context it is important to note that various observations have been made concerning shape-dependent polymeric nanoparticle cell internalization. In this study, poly(Fe^{III}-catecholate)-based nanoparticles were internalized into HeLa cells to a higher extent when they were of a cylindrical morphology than when they were in the form of spherical particles. A similar trend was also reported for shell-crosslinked spherical and cylindrical micelles, as well as polymer brush-based spherical and long rod-like nanostructures. Considering that shape not only plays an important role in cell internalization, but can also be a determining factor in overall biodistribution patterns in vivo (i.e. blood circulation and extravasation), these results demonstrate opportunities for optimization and tunability in the design of self-assembled nanoparticles. This highlights the power of the approach for the preparation of nanoparticles from well-defined polymers generated utilizing a living polymerization. Indeed, as demonstrated herein, the approach taken here provides direct access to two well-defined morphologies of polycatechol-based nanoparticle, not easily achievable utilizing analogous synthetic melanin colloidal nanoparticles prepared by oxidative polymerization.

Conclusion. In summary, we have developed a new class of efficient and biocompatible MRI contrast agents based on micellar nanoparticles formed by amphiphilic poly(Fe(III)-catecholate)-based copolymers. Compared with established natural and synthetic melanin-based T₁ agents, our approach by the use of well-defined amphiphilic tri-block copolymers via a controlled living polymerization method could generate different self-assembled shaped nanoparticles with rigorously controlled physical parameters. These nanoparticles can be used for various applications in diagnostic radiology and imaging, due to their enhanced relaxivity, and long-term stability in biological media. Moreover, we further demonstrate that both nanoparticles provide enhanced contrast of MR imaging in uptake by Hela cells

Experimental

General Methods

Monomer and Polymer Synthesis and Characterization. All chemicals were bought from Sigma-Aldrich and used without further purification, unless otherwise indicated. Anhydrous toluene and dichloromethane were purified using a Dow-Grubbs two-column purification system (Glasscontour System, Irvine, Calif.) [1]. (IMesH₂)(C₅H₅N)₂(Cl)₂Ru=CHPh was prepared as described by Sanford et al [2]. Monomer 1, 2, 3 was synthesized as reported, respectively [3-5]. Polymerizations were performed under a dry dinitrogen atmosphere with anhydrous, degassed solvents in a glove box. Melanin-Fe(III) was prepared as reported [6].

¹H (400 MHz) and ¹³C (100 MHz) NMR spectra were recorded on a Varian Mercury Plus spectrometer. Chemical shifts (¹H) are reported in δ (ppm) relative to the C₅D₅N residual proton peaks (δ 7.22, δ7.58, and δ 8.74 ppm). Chemical shifts (¹³C) are reported in δ (ppm) relative to the C₅D₅N carbon peaks (δ 123.87, δ 135.91, and δ 150.35 ppm). All ¹³C NMR spectra were proton decoupled. Mass spectra were obtained at the UCSD Chemistry and Biochemistry Molecular Mass Spectrometry Facility. Polymer dispersities and molecular weights were determined by size-exclusion chromatography (Phenomenex PHENOGEL™ 5 μ 10, 1K-75K, 300×7.80 mm in series with a Phenomex PHENOGEL™ 5 μ 10, 10K-1000K, 300×7.80 mm (0.05 M LiBr in DMF) using a Shimatzu pump equipped with a multi-angle light scattering detector (DAWN-HELIOS: Wyatt Technology) and a refractive index detector (Optilab T-rEX: Wyatt Technology) normalized to a 30,000 MW polystyrene standard using dn/dc of 0.100 for all the polymers.

Micelles Preparation and Characterization. The samples were first dissolved in THF as the common solvent and stirred at room temperature overnight to ensure complete dissolution of the polymer to prepare a stock solution with an initial concentration of 2 wt %. The solution was then filtered through a filter of 0.22 μm pore size to remove any dust. FeCl₃ solution (1 mg/mL) was filtered through a filter of 0.22 μm pore size and added dropwise at a rate of 10 μL/hour using a syringe pump into a vial containing 2.00 g of the stock solution. FeCl₃ solution addition was continued until a final water content of 70 (wt) %. Then the micelle solution was dialyzed against deionized water for three days to remove the common solvent and excess Fe³⁺ and fix the micellar morphology.

SNAKESKIN™ dialysis tubing was purchased from Thermoscientific, Inc. with a molecular weight cut off (MWCO) of 10,000 g/mol. Hydrodynamic diameter (Dₕ) was determined by DLS using a Wyatt Dynapro NanoStar. Transmission Electron Microcopy (TEM) was performed on a FEI Sphera microscope operating at 200 keV. TEM grids were prepared by depositing small (3.5 μl) aliquots of sample onto grids (˜2 min, Formvar stabilized with carbon (5-10 nm) on 400 copper mesh, Ted Pella Inc.) that had previously been glow discharged using an Emitech K350 glow discharge unit and plasma-cleaned for 90 s in an E.A. Fischione 1020 unit. Micrographs were recorded on a 2K×2K Gatan CCD camera.

Cryo-TEM experiment was also performed on a FEI Sphera microscope operating at 200 keV. TEM grids were prepared by depositing small (3.5 μl) aliquots of sample onto grids Quantifoil R2/2 holey carbon) that had previously been glow discharged using an Emitech K350 glow discharge unit and plasma-cleaned for 90 s in an E.A. Fischione 1020 unit. Sample was loaded onto the grids at 4° C., blotted with filter paper to create a thin film on the grid, then plunged into liquid ethane and transferred into a precooled Gatan 626 cryo-transfer holder, which maintained the specimen at liquid-nitrogen temperature in a FEI Sphera microscope operated at 200 keV. Micrographs were recorded on a 2K×2K Gatan CCD camera.

STEM and STEM-EDS analysis were acquired on a JEOL JEM 2100F TEM equipped with an INCA (Oxford) EDS detector at the NanoScale Fabrication and Characterization Facility (NFCF), Peterson Institute of Nanoscience and Engineering (PINSE), University of Pittsburgh, Pa. Samples were prepared by drop-casting 5 μL of sample onto TEM grids (ultrathin 5 nm A-type carbon with 400 mesh copper, Ted Pella, Inc.) followed by slow drying covered on the bench top for at least 3 hours. Samples were then dried under vacuum for 24-48 hours to remove contamination that would interfere with STEM-EDS. Grids were loaded into a JEOL 31640 beryllium double tilt holder. STEM-EDS data was collected for 180-600 s at specific points, using the largest probe size (1.5 nm electron beam diameter) with a 200 kV accelerating voltage. Images were collected in bright field (BF) and high-angle annular dark field (HAADF) modes.

The magnetic properties of micellar nanoparticles were characterized using a Quantum Design MPMS3 superconducting quantum interference device (SQUID) with a maximum field of 7 T. Freeze-drying solid samples (˜10 mg) were packed into standard Quantum Design plastic sample holders. Magnetization data were collected in DC mode and corrected for diamagnetic contributions using Pascal's constants.

Fe³⁺ Concentration Determination in Micelles. In order to determine Fe³+ concentration, the metal was first stripped from the polymers using the following procedure. To an aliquot of each sample (100 μL) was added 1% HNO₃ in water (1900 μL). Each mixture was then stirring for about 12 hours. Then the Fe³⁺ concentration was quantified by inductively coupled plasma-optical emission spectrometry (ICP-OES) using a Perkin Elmer Optima 3000DV spectrometer in the Scripps Institution of Oceanography, University of California, San Diego.

Fe³⁺ Stability in PBS. To determine the stability of Fe³⁺ chelated in SMN and CMN, we re-dispersed these two types of micellar nanoparticles in PBS(pH=7.4). 300 ul of SMN and CMN solution (three replicates) were added in 500 ul dialysis tubes with Mw=3500 respectively, and dialyzed to 500 ml PBS(pH=7.4) under room temperature with magnetic stirring. 20 ul SMN and CMN aliquots were taken at time points as 8 h, 24 h, 48 h, 72 h, 7 days for ICP-OES analysis.

Determination of the In Vitro Stability of Micelles in FBS by MRI Analysis. Samples of both SMN and CMN were prepared in FBS at various [FeIII] concentrations (For SMN, 5 [FeIII] concentrations were used: 0.6 mM, 0.3 mM, 0.15 mM, 0.075 mM, and 0.038 mM; while for CMN, 5 [FeIII] concentrations were used: 0.67 mM, 0.34 mM, 0.17 mM, 0.08 mM, and 0.04 mM) 3 days prior to MRI analysis, and as controls, samples of identical concentration to the latter were prepared in both FBS and water immediately before MRI analysis (named, SMN/CMN in FBS for 3 days, SMN/CMN in freshly prepared FBS, and SMN/CMN in water, respectively). Longitudinal and transverse relaxation time (T₁ and T₂) measurements were acquired with a contrast agent analyzer (a Time Domain-NMR instrument, Bruker, Minispec mq60, 1.41T/60 MHz, 37° C.). Relaxivities r₁(r₂) were calculated after the curve fitting of 1/T₁ (s⁻¹) (or 1/T₂(s⁻¹)) versus Fe(III) ions concentrations (μM). The MR images were acquired on a Bruker 7.0T magnet with Avance II hardware equipped with a 72 mm quadrature transmit/receive coil. T₁ contrast was determined by selecting regions of interest (ROI) using Software ParaVision Version 5.1. The parameters for 7T MRI are: TR=750.0 ms, TE=12.6 ms, echo=1/1, FOV=6.91/3.12 cm, slice thickness=2 mm, nex=2 mm, matrix=256*116.

¹H NMRD Profiles. Proton 1/T₁ NMRD profiles were measured on a fast field-cycling Stelar SMARTracer Relaxometer (Stelar, Mede (PV), Italy) at magnetic field strengths from 0.00024 to 0.25 T (corresponding to 0.01-10 MHz proton Larmor frequencies) at room temperature. The relaxometer operates under computer control with an absolute uncertainty in 1/T₁ of ±1%. Additional data points in the range 15-70 MHz were obtained on a Bruker WP80 NMR electromagnet adapted to variable-field measurements (15-80 MHz proton Larmor frequency) Stelar Relaxometer. The ¹H T₁ relaxation times were acquired by the standard inversion recovery method with typical 90° pulse width of 3.5 μs, 16 experiments of 4 scans. The temperature was controlled with a Stelar VTC-91 airflow heater equipped with a calibrated copper—constantan thermocouple (uncertainty of ±0.1° C.).

Cell Viability. In vitro cytotoxicity of micellar nanoparticles was determined in Hela cells by the CCK-8 (cell counting kit-8) assay. Hela cells were incubated on 96-well plates with 1*10⁴ cells per well in High-glucose DMEM medium containing 10% fetal bovine serum and 1% antibiotics at 37° C. in 5% CO₂ humidified atmosphere for 24 h and 48 h respectively. Addition of 10 ul of CCK-8 solution to each well and incubation for another 4 hours at 37° C. were followed to produce formazan crystals. Then the absorbance value at 460 nm was recorded using a microplate reader. The absorbance value of the untreated cells was used as the reference value of 100% cellular viability.

Shape- and Time-Dependent MR Imaging in Hela cells. Hela cells were seeded in 15 cm round tissue culture dishes and allowed to attach overnight. After washing twice by sterile PBS, the cells were incubated with micellar nanoparticles (the concentrations of Fe(III) ions were approximately 67.5 μM) for different time including 4 hours, 12 hours, 24 hours, and 48 hours respectively at normal cell culture condition. The cells were washed by PBS for three times in order to remove excessive nanoparticles, and then were treated with 0.05% trypsin to remove from dishes. The cells were gathered by centrifuge at 300 g for 3 min and washed by PBS buffer twice. The cells number in each sample was counted for further use. The cell MR images were acquired on a Bruker 7.0T magnet with Avance II hardware equipped with a 72 mm quadrature transmit/receive coil. T₁ contrast was determined by selecting regions of interest (ROI) using Software ParaVision Version 5.1. The parameters for 7T MRI are: TR=1000.0 ms, TE=12.6 ms, echo length=1, FOV=7.91/3.22 cm, slice thickness=1 mm, nex=1 mm, matrix=256*104. Then, the cells were digested by 70% HNO₃ solution under bath sonication for overnight, in order to test the iron ions content by ICP-MS. The Fe(III) quantities of each samples were normalized to 10⁶ cells.

Cell TEM Observation. Hela cells were seeded in 35 mm round tissue culture dishes and allowed to proliferate until 80% fluent. After washing twice by sterile PBS buffer, the cells were incubated with micellar nanoparticles (the concentrations of Fe(III) ions were approximately 67.5 μM) for 24 hours at 37° C. The cells were washed by PBS for three times in order to remove excessive nanoparticles, and then were fixed by 2% glutaraldehyde in 0.1M sodium cacodylate buffer with pH=7.4 (SC buffer) on ice for more than 2 hours. After washing three times with 0.1M SC buffer for 5 min each, the cells were postfixed with 1% osmium tetroxide in 0.1M SC buffer for 1 hour on ice. Then cell pellets were washed by 0.1M SC buffer for 3 time and 5 min each, followed by a quick rinse in H₂O. Then cell pellets were stained by 2% uranyl acetate (UA) for 1 hour on ice. After staining, they were dehydrated in a graded series of ethanol (50%, 70%, 90% and 100%) for 5-8 min each, and dried in acetone at room temperature. Then, cell pellets were infiltrated by 50:50 dry acetone/durcupan for 1-2 hours on rotator, followed by 100% durcupan overnight and 2× 100% durcupan next day. Finally cell pellets were embedded in durcupan and incubated in oven at 60° C. for 36-48 hours. Ultrathin sections were cut and were examined using an electron microscopy.

NMRD Profiles Analysis. Simplified model was utilized to analyze the data and obtain the estimation for some relevant molecular parameters affecting the relaxivity of the system. In this model, only the high field data were considered and the fit was performed according to the Solomon-Bloembergen-Morgan (SBM) set of equations, as discussed in the main text. The determination of electron spin relaxation parameters is almost entirely dependent upon fitting the low field data. As we stated in the manuscript, low field data was not included in the fitting. The reason for this is simply that SBM theory does not function very well across this frequency range for slowly tumbling systems—a fact that has been commonly noted for many years by several different groups [1]. For this reason it would be unwise to try to attribute any genuine physical meaning to the values of Δ² and τᵥ. We then used as adjustable parameters Δ², τᵥ, τ_{R}, τ_{M} and q/r⁶. Satisfactory fit was obtained with the parameters reported in Table 1-1. The choice of q=2 is arbitrary and was made since associated with the reasonable value of r of 3.3 A. Of course, setting q=3 we would obtain values for r equal to 3.72 Å. The same value was reported in the case of Fe(III)heme-HSA [2]. A distance of 3.2±0.1 Å was also reported for bovine lactoferrin [3]. The value of the rotational correlation times is much shorter than that associated with a macromolecular system and in good agreement with that expected for loosely bound second sphere water molecules. The exchange lifetime τ_{M} is also very similar to that estimated for bovine lactoferrin [3].

Synthetic Procedures.

Synthesis of (OEG)₃₈-(NHS)₃₄-(C₆)₅₀. To stirred 3.7 mL DMF solution of 1 (194 mg, 0.548 mmol) was added a solution of the initiator (IMesH₂)(C₅H₅N)₂(Cl)₂Ru=CHPh (10 mg, 0.0137 mmol) in DMF (0.3 mL). The reaction was left to stir in the glove box for 30 min, after which an analytical aliquot (0.1 mL) was removed and mixed with an excess of ethyl vinyl ether for 30 min, then dried under high vacuum to give a homopolymer of 1. To the remaining reaction mixture, 0.5 mL DMF solution of 2 (97 mg, 0.411 mmol) was then added immediately following analytical aliquot removal. The reaction was stirred in the glove for another 30 min, after which an analytical aliquot (0.1 mL) was removed and mixed with an excess of ethyl vinyl ether for 30 min, then dried under high vacuum to give a block copolymer of 1-b-2. Again to the remaining reaction mixture, 0.5 mL DMF solution of 3 (170 mg, 0.685 mmol) was then added immediately following analytical aliquot removal. The mixture was left to stir in the glove box for 40 min and then quenched with excess ethyl vinyl ether (0.5 mL) for about 20 min, which was concentrated to dryness to give a yellow solid as the final product (392 mg, 85%).

SEC-MALS of each polymer:
Homopolymer of 1: M_{n, SEC}=13.4 kg/mol, M_{w, SEC}=13.9 kg/mol, PDI=1.04, N(OEG)=38.
Copolymer of 1-b-2: M_{n, SEC}=21.4 kg/mol, M_{w, SEC}=23.3 kg/mol, PDI=1.09, N(NHS)=34.
(OEG)₃₈-(NHS)₃₄-(C₆)₅₀: M_{n, SEC}=33.8 kg/mol, M_{w, SEC}=37.1 kg/mol, PDI=1.10, N(C₆)=50.

Synthesis of (OEG)₂₀-(NHS)₂₃-(C₆)₄₃. To stirred 3.7 mL DMF solution of 1 (97 mg, 0.275 mmol) was added a solution of the initiator (IMesH₂)(C₅H₅N)₂(Cl)₂Ru=CHPh (10 mg, 0.0137 mmol) in DMF (0.3 mL). The reaction was left to stir in the glove box for 30 min, after which an analytical aliquot (0.1 mL) was removed and mixed with an excess of ethyl vinyl ether for 30 min, then dried under high vacuum to give a homopolymer of 1. To the remaining reaction mixture, 0.5 mL DMF solution of 2 (65 mg, 0.277 mmol) was then added immediately following analytical aliquot removal. The reaction was stirred in the glove for another 30 min, after which an analytical aliquot (0.1 mL) was removed and mixed with an excess of ethyl vinyl ether for 30 min, then dried under high vacuum to give a block copolymer of 1-b-2. Again to the remaining reaction mixture, 0.5 mL DMF solution of 3 (136 mg, 0.551 mmol) was then added immediately following analytical aliquot removal. The mixture was left to stir in the glove box for 40 min and then quenched with excess ethyl vinyl ether (0.5 mL) for about 20 min, which was concentrated to dryness to give a yellow solid as the final product (241 mg, 81%).

SEC-MALS of each polymer:
Homopolymer of 1: M_{n, SEC}=7.1 kg/mol, M_{w, SEC}=7.3 kg/mol, PDI=1.03, N(OEG)=20.
Copolymer of 1-b-2: M_{n, SEC}=12.6 kg/mol, M_{w, SEC}=13.6 kg/mol, PDI=1.08, N(NHS)=23.
(OEG)₂₀-(NHS)₂₃-(C₆)₄₃: M_{n, SEC}=23.1 kg/mol, M_{w, SEC}=25.4 kg/mol, PDI=1.09, N(C₆)=43.

Synthesis of (OEG)₃₈-(Cat)₃₄-(C₆)₅₀ (Polymer 1). (OEG)₃₈-(NHS)₃₄-(C₆)₅₀ (M_{n, SEC}=33.7 kg/mol, 300 mg, 0.0089 mmol), 3-hydroxytyramine hydrochloride (287 mg, 1.51 mmol), and N-Ethyldiisopropylamine (390 mg, 3.02 mmol) were fully dissolved in 10 mL of DMF. The mixture was stirred at room temperature for about 2 days. The solution was then precipitated into 1M HCl solution three times. The brown precipitation was then filtered off and redissolved into 10 mL THF. The solution was again precipitated into cold ether three times. The final product was collected and dried under vacuum overnight to afford a brown powder (268 mg; Yield: 86%) as Polymer 1.

Synthesis of (OEG)₂₀-(Cat)₂₃-(C₆)₄₃ (Polymer 2). (OEG)₃₈-(NHS)₃₄-(C₆)₅₀ (M_{n, SEC}=23.1 kg/mol, 180 mg, 0.0078 mmol), 3-hydroxytyramine hydrochloride (155 mg, 0.82 mmol), and N-Ethyldiisopropylamine (211 mg, 1.64 mmol) were fully dissolved in 10 mL of DMF. The mixture was stirred at room temperature for about 2 days. The solution was then precipitated into 1M HCl solution three times. The brown precipitation was then filtered off and redissolved into 8 mL THF. The solution was again precipitated into cold ether three times. The final product was collected and dried under vacuum overnight to afford a brown powder (153 mg; Yield: 82%) as Polymer 2.

Results of Cambridge Structural Database (CSD) Search for Hydrogen-Bonding Interactions between Polar H—X Bonds and Fe^{III}(Catecholate)₃ Cores. To provide additional evidence for the viability of second-sphere interactions between H₂O and paramagnetic Fe^{III}(catecholate)₃ cores, two Cambridge Structural Database (CSD) [7-9] searches were conducted using the search criteria set forth in Scheme 1 following.

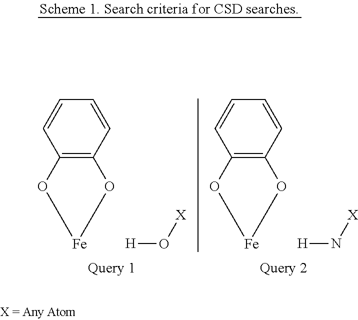

A total of 72 unique structures were found featuring hydrogen-bonding interactions between the oxygen atoms of the Fe^{III}catecholate)₃ primary coordination sphere and polar H—O or H—N bonds. Examples of both intra- and intermolecular hydrogen bonding interactions were found. The selected examples referenced in the text are shown in FIGS. 17-21 along with CSD codes.

### REFERENCES (EXAMPLE 1—EXPERIMENTAL SECTION)

[1] Pangborn, A. B.; Giardello, M. A.; Grubbs, R. H.; Rosen, R. K.; Timmers, F. J. Organometallics 1996, 15, 1518-1520; [2] Sanford, M. S.; Love, J. A.; Grubbs, R. H. Organometallics 2001, 20, 5314-5318; [3] Hahn, M. E.; Randolph, L. M.; Adamiak, L.; Thompson, M. P.; Gianneschi, N. C. Chem. Commun. 2013, 49, 2873-2875; [4] Ku, T.-H.; Chien, M.-P.; Thompson, M. P.; Sinkovits, R. S.; Olson, N. H.; Baker, T. S.; Gianneschi, N. C. J. Am. Chem. Soc. 2011, 133, 8392-8395; [5] Culberson, C. F.; Wilder Jr., P. J. Org. Chem. 1960, 25, 1358-1362; [6] Ju, K.-Y.; Lee, J. W.; Im, G. H.; Lee, S.; Pyo, J.; Park, S. B.; Lee, J. H.; Lee, J.-K. Biomacromolecules 2013, 14, 3491; [7] Cambridge Structural Database (CSD), version 5.34 (Update 2, Feb. 2013); [8] Allen, F. Acta Crystallogr. B 2002, 58, 380; [9] Allen, F. H.; Motherwell, W. D. S. Acta Crystallogr. B 2002, 58, 407; [10] Anderson, B. F.; Buckingham, D. A.; Robertson, G. B.; Webb, J.; Murray, K. S.; Clark, P. E. Nature 1976, 262, 722-724; [11] Anderson, B. F.; Buckingham, D. A.; Robertson, G. B.; Webb, J. Acta Crystallogr. B 1982, 38, 1927-1931; [12] McMurry, T. J.; Hosseini, M. W.; Garrett, T. M.; Jahn, F. E.; Reyes, Z. E.; Raymond, K. N. J. Am. Chem. Soc. 1987, 109, 7196-7198; [13] Karpishin, T. B.; Stack, T. D. P.; Raymond, K. N. J. Am. Chem. Soc. 1993, 115, 6115-6125; [14] Jurchen, K. M. C.; Raymond, K. N. J. Coord. Chem. 2005, 58, 55-80; [15] Panja, A. Inorg. Chem. Commun. 2013, 32, 42-46.

### VII. REFERENCES (EXAMPLE 1)

[1] Fox, M. D.; Raichle, M. E., Spontaneous fluctuations in brain activity observed with functional magnetic resonance imaging. Nature Reviews Neuroscience 2007, 8, 700-711; [2] Terreno, E.; Castelli, D. D.; Viale, A.; Aime, S., Challenges for molecular magnetic resonance imaging. Chemical Reviews 2010, 110, 3019-3042; [3] Na, H. B.; Song, I. C.; Hyeon, T., Inorganic nanoparticles for MRI contrast agents. Advanced Materials 2009, 21, 2133-2148; [4] Laurent, S.; Forge, D.; Port, M.; Roch, A.; Robic, C.; Vander Elst, L.; Muller, R. N., Magnetic iron oxide nanoparticles: Synthesis, stabilization, vectorization, physicochemical characterizations and biological applications. Chemical Reviews 2008, 108, 2064-2110; [5] Lauffer, R. B., Paramagnetic metal complexes as water proton relaxation agents for NMR imaging: Theory and design. Chemical Reviews 1987, 87, 901-927; [6] Caravan, P.; Ellison, J. J.; McMurry, T. J.; Lauffer, R. B., Gadolinium(III) chelates as MRI contrast agents: Structure, dynamics, and applications. Chemical Reviews 1999, 99, 2293-2352; [7] Caravan, P., Strategies for increasing the sensitivity of gadolinium based MRI contrast agents. Chemical Society Reviews 2006, 35, 512-523; [8] Aime, S.; Botta, M.; Terreno, E., Gd(III)-BASED CONTRAST AGENTS FOR MRI. Advances in Inorganic Chemistry 2006, 57, 173-237; [9] Aime, S.; Caravan, P., Biodistribution of gadolinium-based contrast agents, including gadolinium deposition. Journal of Magnetic Resonance Imaging 2009, 30, 1259-1267; [10] Kribben, A.; Witzke, O.; Hillen, U.; Barkhausen, J.; Daul, A. E.; Erbel, R., Nephrogenic Systemic Fibrosis. Pathogenesis, Diagnosis, and Therapy. Journal of the American College of Cardiology 2009, 53, 1621-1628; [11] Cotton, F. A.; Wilkinson, G.; Murillo, C. A.; Bochmann, M., Advanced Inorganic Chemistry. John Wiley and Sons, New York. 1999; [12] Ju, K.-Y.; Lee, J. W.; Im, G. H.; Lee, S.; Pyo, J.; Park, S. B.; Lee, J. H.; Lee, J.-K., Bio-inspired, melanin-like nanoparticles as a highly efficient contrast agent for T 1-weighted magnetic resonance imaging. Biomacromolecules 2013, 14, 3491-3497; [13] Lee, B. P.; Messersmith, P. B.; Israelachvili, J. N.; Waite, J. H., Mussel-inspired adhesives and coatings. Annual Review of Materials Research 2011, 41, 99-132; [14] Harrington, M. J.; Masic, A.; Holten-Andersen, N.; Waite, J. H.; Fratzl, P., Iron-clad fibers: A metal-based biological strategy for hard flexible coatings. Science 2010, 328, 216-220; [15] Schwert, D. D.; Richardson, N.; Ji, G.; Radüchel, B.; Ebert, W.; Heffner, P. E.; Keck, R.; Davies, J. A., Synthesis of two 3,5-disubstituted sulfonamide catechol ligands and evaluation of their iron(III) complexes for use as MRI contrast agents. Journal of Medicinal Chemistry 2005, 48, 7482-7485; [16] Tang, J.; Sheng, Y.; Hu, H.; Shen, Y., Macromolecular MRI contrast agents: Structures, properties and applications. Progress in Polymer Science 2013, 38, 462-502; [17] Liu, Y.; Ai, K.; Lu, L., Polydopamine and its derivative materials: Synthesis and promising applications in energy, environmental, and biomedical fields. Chemical Reviews 2014, 114, 5057-5115; [18] Lynge, M. E.; Van Der Westen, R.; Postma, A.; Städler, B., Polydopamine—A nature-inspired polymer coating for biomedical science. Nanoscale 2011, 3, 4916-4928; [19] Ye, Q.; Zhou, F.; Liu, W., Bioinspired catecholic chemistry for surface modification. Chemical Society Reviews 2011, 40, 4244-4258; [20] d'Ischia, M.; Napolitano, A.; Ball, V.; Chen, C.-T.; Buehler, M. J., Polydopamine and Eumelanin: From Structure—Property Relationships to a Unified Tailoring Strategy. Acc. Chem. Res. 2014, 47, 3541-3550; [21] Faure, E.; Falentin-Daudré, C.; Jérôme, C.; Lyskawa, J.; Fournier, D.; Woisel, P.; Detrembleur, C., Catechols as versatile platforms in polymer chemistry. Progress in Polymer Science 2013, 38, 236-270; [22] Lee, H.; Dellatore, S. M.; Miller, W. M.; Messersmith, P. B., Mussel-inspired surface chemistry for multifunctional coatings. Science 2007, 318, 426-430; [23] Caulder, D. L.; Raymond, K. N., Supermolecules by design. Accounts of Chemical Research 1999, 32, 975-982; [24] Enochs, W. S.; Petherick, P.; Bogdanova, A.; Mohr, U.; Weissleder, R., Paramagnetic metal scavenging by melanin: MR imaging. Radiology 1997, 204, 417-423; [25] Fan, Q.; Cheng, K.; Hu, X.; Ma, X.; Zhang, R.; Yang, M.; Lu, X.; Xing, L.; Huang, W.; Gambhir, S. S.; Cheng, Z., Transferring Biomarker into Molecular Probe: Melanin Nanoparticle as a Naturally Active Platform for Multimodality Imaging. J. Am. Chem. Soc. 2014, 136, 15185-15194; [26] Ku, T.-H.; Chien, M.-P.; Thompson, M. P.; Sinkovits, R. S.; Olson, N. H.; Baker, T. S.; Gianneschi, N. C., Controlling and switching the morphology of micellar nanoparticles with enzymes. Journal of the American Chemical Society 2011, 133, 8392-8395; [27] Rush, A. M.; Nelles, D. A.; Blum, A. P.; Barnhill, S. A.; Tatro, E. T.; Yeo, G. W.; Gianneschi, N. C., Intracellular mRNA regulation with self-assembled locked nucleic acid polymer nanoparticles. Journal of the American Chemical Society 2014, 136, 7615-7618; [28] Chien, M.-P.; Rush, A. M.; Thompson, M. P.; Gianneschi, N. C., Programmable shape-shifting micelles. Angewandte Chemie—International Edition 2010, 49, 5076-5080; [29] Chien, M.-P.; Thompson, M. P.; Barback, C. V.; Ku, T.-H.; Hall, D. J.; Gianneschi, N. C., Enzyme-directed assembly of a nanoparticle probe in tumor tissue. Advanced Materials 2013, 25, 3599-3604; [30] Proetto, M. T.; Rush, A. M.; Chien, M.-P.; Abellan Baeza, P.; Patterson, J. P.; Thompson, M. P.; Olson, N. H.; Moore, C. E.; Rheingold, A. L.; Andolina, C.; Millstone, J.; Howell, S. B.; Browning, N. D.; Evans, J. E.; Gianneschi, N. C., Dynamics of soft nanomaterials captured by transmission electron microscopy in liquid water. Journal of the American Chemical Society 2014, 136, 1162-1165; [31] Geng, Y.; Dalhaimer, P.; Cai, S.; Tsai, R.; Tewari, M.; Minko, T.; Discher, D. E., Shape effects of filaments versus spherical particles in flow and drug delivery. Nature Nanotechnology 2007, 2, 249-255; [32] Champion, J. A.; Katare, Y. K.; Mitragotri, S., Particle shape: A new design parameter for micro- and nanoscale drug delivery carriers. Journal of Controlled Release 2007, 121, 3-9; [33] Bielawski, C. W.; Grubbs, R. H., Living ring-opening metathesis polymerization. Progress in Polymer Science 2007, 32, 1-29; [34] Wang, Z.; Li, Y.; Dong, X.-H.; Yu, X.; Guo, K.; Su, H.; Yue, K.; Wesdemiotis, C.; Cheng, S. Z. D.; Zhang, W.-B., Giant gemini surfactants based on polystyrene-hydrophilic polyhedral oligomeric silsesquioxane shape amphiphiles: Sequential “click” chemistry and solution self-assembly. Chemical Science 2013, 4, 1345-1352; [35] Mai, Y.; Eisenberg, A., Self-assembly of block copolymers. Chem. Soc. Rev., 2012, 41, 5969-5985; [36] Lauffer, R. B.; Greif, W. L.; Stark, D. D.; Vincent, A. C.; Saini, S.; Wedeen, V. J.; Brady, T. J., Iron-EHPG as an hepatobiliary MR contrast agent: Initial imaging and biodistribution studies. Int J Rad Appl Instrum B 1988, 15, 47-49; [37] Botta, M., Second coordination sphere water molecules and relaxivity of gadolinium(III) complexes: Implications for MRI contrast agents. European Journal of Inorganic Chemistry 2000, 399-407; [38] Stack, T. D. P.; Karpishin, T. B.; Raymond, K. N., Structural and spectroscopic characterization of chiral ferric tris-catecholamides: unraveling the design of enterobactin. J. Am. Chem. Soc. 1992, 114, 1512-1514; [39] Brückner, C.; Caulder, D. L.; Raymond, K. N., Preparation and Structural Characterization of Nickel(II) Catecholates. Inorganic Chemistry 1998, 37, 6759-6764; [40] Kabir, M. K.; Kawahara, M.; Kumagai, H.; Adachi, K.; Kawata, S.; Ishii, T.; Kitagawa, S., The rational syntheses of manganese-chloranilate compounds: Crystal structures and magnetic properties. Polyhedron 2001, 20, 1417-1422; [41] Fasano, M.; Fanali, G.; Polticelli, F.; Ascenzi, P.; Antonini, G., 1H NMR relaxometric characterization of bovine lactoferrin. Journal of Inorganic Biochemistry 2004, 98, 1421-1426; [42] Koenig, S. H.; Brown III, R. D.; Lindstrom, T. R., Interactions of solvent with the heme region of methemoglobin and fluoro-methemoglobin. Biophysical Journal 1981, 34, 397-408; [43] Xu, H.; Regino, C. A. S.; Bernardo, M.; Koyama, Y.; Kobayashi, H.; Choyke, P. L.; Brechbiel, M. W., Toward improved syntheses of dendrimer-based magnetic resonance imaging contrast agents: New bifunctional diethylenetriaminepentaacetic acid ligands and nonaqueous conjugation chemistry. Journal of Medicinal Chemistry 2007, 50, 3185-3193; [44] Ananta, J. S.; Godin, B.; Sethi, R.; Moriggi, L.; Liu, X.; Serda, R. E.; Krishnamurthy, R.; Muthupillai, R.; Bolskar, R. D.; Helm, L.; Ferrari, M.; Wilson, L. J.; Decuzzi, P., Geometrical confinement of gadolinium-based contrast agents in nanoporous particles enhances T1 contrast. Nature Nanotechnology 2010, 5, 815-821; [45] Fries, P. H.; Belorizky, E., Electronic relaxation of paramagnetic metal ions and NMR relaxivity in solution: Critical analysis of various approaches and application to a Gd(III)-based contrast agent. Journal of Chemical Physics 2005, 123, 124510; [46] Davies, J. A.; Dutremez, S. G.; Hockensmith, C. M.; Keck, R.; Richardson, N.; Selman, S.; Smith, D. A.; Ulmer, C. W.; Wheatley, L. S.; Zeiss, J., Iron-based second-sphere contrast agents for magnetic resonance imaging: development of a model system and evaluation of iron (III) tris (tironate) complex in rats. Academic radiology 1996, 3, 936-945; [47] Barkey, N. M.; Preihs, C.; Cornnell, H. H.; Martinez, G.; Carie, A.; Vagner, J.; Xu, L.; Lloyd, M. C.; Lynch, V. M.; Hruby, V. J.; Sessler, J. L.; Sill, K. N.; Gillies, R. J.; Morse, D. L., Development and in vivo quantitative magnetic resonance imaging of polymer micelles targeted to the melanocortin 1 receptor. Journal of Medicinal Chemistry 2013, 56, 6330-6338; [48] Yang, J.; Stuart, M. A. C.; Kamperman, M., Jack of all trades: versatile catechol crosslinking mechanisms. Chem. Soc. Rev., 2014, 43, 8271-8298; [49] Holten-Andersen, N.; Jaishankar, A.; Harrington, M. J.; Fullenkamp, D. E.; Dimarco, G.; He, L. d.; McKinley, G. H.; Messersmith, P. B.; Lee, K. Y. C., Metal-coordination: Using one of nature's tricks to control soft material mechanics. Journal of Materials Chemistry B 2014, 2, 2467-2472; [50] Holten-Andersen, N.; Harrington, M. J.; Birkedal, H.; Lee, B. P.; Messersmith, P. B.; Lee, K. Y. C.; Waite, J. H., pH-induced metal-ligand cross-links inspired by mussel yield self-healing polymer networks with near-covalent elastic moduli. Proceedings of the National Academy of Sciences of the United States of America 2011, 108, 2651-2655; [51] Ejima, H.; Richardson, J. J.; Liang, K.; Best, J. P.; Van Koeverden, M. P.; Such, G. K.; Cui, J.; Caruso, F., One-step assembly of coordination complexes for versatile film and particle engineering. Science 2013, 341, 154-157; [52] Sadler, N. P.; Chuang, C.-C.; Milburn, R. M., Iron(III)-promoted hydrolysis of 4-nitrophenyl phosphate. Inorganic Chemistry 1995, 34, 402-404; [53] Tao, L.; Hu, W.; Liu, Y.; Huang, G.; Sumer, B. D.; Gao, J., Shape-specific polymeric nanomedicine: Emerging opportunities and challenges. Experimental Biology and Medicine 2011, 236, 20-29; [54] Zhang, K.; Rossin, R.; Hagooly, A.; Chen, Z.; Welch, M. J.; Wooley, K. L., Folate-mediated cell uptake of shell-crosslinked spheres and cylinders. Journal of Polymer Science, Part A: Polymer Chemistry 2008, 46, 7578-7583.

### Example 2

### In Vitro and In Vivo MRI and Analysis of T₁ Data

Experimental. MRI instrumental set-up details and parameters can be as described above. MR images can be acquired on a Bruker 7.0 T magnet.

Female mice (C57B1/6) weighing 18 grams can be purchased from Harlan Sprague Dawley and can be anesthetized with 3% isoflurane in O2 and subjected to baseline MRI imaging before injection. A total of nine mice (three sets of three) can be injected with 550 μL of an aqueous 0.4 mM Gd-DOTA, SMN, or CMN solution intraperitoneally and imaged continuously under anesthesia for two hours and then again at selected time points of approximately 3 h, 4 h, 5 h, 6 h, 7 h, 8 h, 24 h, 48 h, and 1 week (each measurement made under anesthesia). To correct for minor scan-to-scan variations due to noise, T1 can be normalized to pre-injection phantom relaxivities.

Mice can be sacrificed using a lethal overdose of >5% isofluorane and selected organs harvested. The liver, bowel and spleen can be dissected and placed in separate tubes, and their masses can be recorded individually. Mass of the entire liver, bowel and spleen can be recorded, added to concentrated nitric acid (900 mL), and placed on a shaker overnight with venting. The following morning, concentrated H₂O₂ (50 μL) can be added to each of the organ solutions and placed back on the shake with venting for approximately 30 min. An aliquot (e.g., 200 μL) of the digested organs can be added to distilled DI water (800 μL) and submitted to Exova for ICP-MS analysis to determine Gd³⁺ or Fe³⁺ concentration. The final concentration of Gd³⁺ or Fe³⁺ in each organ can be normalized to organ mass.

1. A polymeric micellar nanoparticle, wherein said polymeric micellar nanoparticle comprises a Fe(III)-catecholate complex.

2. The polymeric micellar nanoparticle of claim 1, wherein said polymeric micellar nanoparticle comprises a plurality of block copolymers, wherein each block copolymer comprises a first block of hydrophobic polymerized monomers, a second block of catechol polymerized monomers and optionally a third block of hydrophilic polymerized monomers, wherein:
the first block of hydrophobic polymerized monomers comprise a hydrophobic moiety covalently attached to each first block monomer backbone moiety within said first block of hydrophobic polymerized monomers, wherein each hydrophobic moiety is optionally different;
(ii) the second block of catechol polymerized monomers comprise a catechol moiety covalently attached to each second block monomer backbone moiety within said second block of catechol polymerized monomers, wherein at least one catechol moiety is complexed to iron thereby forming said Fe(III)-catecholate complex; and
(iii) the third block of hydrophilic polymerized monomers comprise a hydrophilic moiety covalently attached to each third block monomer backbone moiety within said third block of hydrophilic polymerized monomers, wherein each hydrophilic moiety is optionally different.

3. (canceled)

4. The polymeric micellar nanoparticle of claim 2 of the formula:
R¹-L¹-(A(-L²-R²))_{z1}(B(-L³-R³))_{z2}-[(C(-L⁴-R⁴))_{z3}]_{z5}-L⁵-R⁵
wherein
(A(-L²-R²))_{z1} is the first block of hydrophobic polymerized monomers;
(B(-L³-R³))_{z2} is the second block of catechol polymerized monomers;
(C(-L⁴-R⁴))_{z}3 is the third block of hydrophilic polymerized monomers;
A is a first block monomer backbone moiety;
B is a second block monomer backbone moiety;
C is a third block monomer backbone moiety;
z1, z2 and z3 are independently integers from 1 to 100;
z5 is 0 or 1;
L¹ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
L²-R² is a hydrophobic moiety, wherein
L² is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R² is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
L³-R³ is a catechol moiety, wherein
L³ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is substituted or unsubstituted 1,2-dihydroxyphenyl;
L⁴-R⁴ is a hydrophilic moiety, wherein
L⁴ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
L⁵ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R⁵ is hydrogen, —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

5. (canceled)

6. The polymeric micellar nanoparticle of claim 4, wherein L³-R³ is:

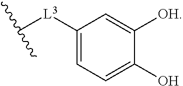

7. The polymeric micellar nanoparticle of claim 6, wherein L³ is —C(O)—N(H)—(CH2)_{z4}- wherein z4 is 1 to 10.

8. (canceled)

9. (canceled)

10. (canceled)

11. (canceled)

12. (canceled)

13. The polymeric micellar nanoparticle of claim 4, wherein (A(-L²-R²))_{z1} has the formula (-L^{1A}-A¹(-L²-R²)-L^{2A}-)_{z1}, wherein
L^{1A} and L^{2A} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
A¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

14. The polymeric micellar nanoparticle of claim 4, wherein (B(-L³-R³))_{z2} has the formula (-L^{1B}-B¹(-L³-R³)-L^{2B}-)_{z2}, wherein
L^{1B} and L^{2B} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
B¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

15. The polymeric micellar nanoparticle of claim 4, wherein (C(-L⁴-R⁴))_{z3} has the formula (-L^{1C}-C¹(-L⁴-R⁴)-L^{2C}-)_{z3}, wherein
L^{1C} and L^{2C} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
C¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

16. The polymeric micellar nanoparticle of claim 4, wherein (B(-L³-R³))_{z2} has the formula: 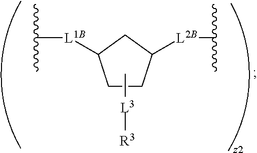
wherein L^{1B} and L^{2B} are independently a bond, —O—, —S—, —NH—, —C(O)—, —C(O)O—, —C(O)NH—, substituted or unsubstituted alkylene, substituted or unsubstituted heteroalkylene.

17. The polymeric micellar nanoparticle of claim 4, wherein (A(-L²-R²))_{z1} has the formula: 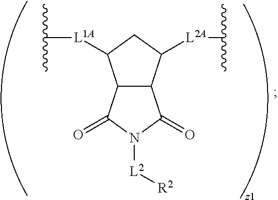
wherein L^{1A} and L^{2A} are independently a bond, —O—, —S—, —NH—, —C(O)—, —C(O)O—, —C(O)NH—, substituted or unsubstituted alkylene, substituted or unsubstituted heteroalkylene.

18. The polymeric micellar nanoparticle of claim 4, wherein (C(-L⁴-R⁴))_{z3} has the formula: 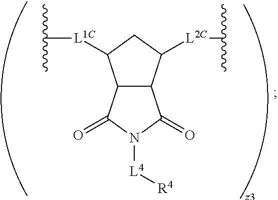
wherein L^{1C} and L^{2C} are independently a bond, —O—, —S—, —NH—, —C(O)—, —C(O)O—, —C(O)NH—, substituted or unsubstituted alkylene, substituted or unsubstituted heteroalkylene.

19. (canceled)

20. (canceled)

21. (canceled)

22. The polymeric micellar nanoparticle of claim 4, wherein L²-R² is 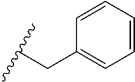 [SMILES: *Cc1ccccc1]
is or an unsubstituted C₁-C₁₀ alkyl.

23. (canceled)

24. The polymeric micellar nanoparticle of claim 4, wherein L⁴-R⁴ is polyethylene glycol.

25. (canceled)

26. (canceled)

27. (canceled)

28. (canceled)

29. (canceled).

30. The polymeric micellar nanoparticle of claim 1, wherein said polymeric micellar nanoparticle is prepared from amphiphilic block copolymers.

31. (canceled)

32. (canceled)

33. (canceled)

34. (canceled)

35. The polymeric micellar nanoparticle of claim 1, wherein said polymeric micellar nanoparticle has a relaxivity value (r₁) of about 7×10⁻³ M⁻¹ S⁻¹ to about 10×10^{31 3} M⁻¹ S⁻¹.

36. A method of providing visibility of an internal body structure of a subject undergoing MRI, the method comprising administering to said subject an effective amount of the polymeric micellar nanoparticle of claim 1.

37. (canceled)

38. A method of detecting an internal body structure of a subject, the method comprising administering to said subject an effective amount of the polymeric micellar nanoparticle of claim 1 and detecting said polymeric micellar nanoparticle using magnetic resonance imaging thereby detecting said internal body structure.

39. (canceled)

40. A method for synthesizing a polymeric micellar nanoparticle, said method comprising
functionalizing a block copolymer amphiphile to incorporate a catechol group in one block of said block copolymer amphiphile, thereby providing a functionalized block copolymer amphiphile;
contacting said functionalized block copolymer amphiphile under conditions suitable to afford a catechol-containing block copolymer; and
contacting said catechol-containing block copolymer with a metal salt, thereby providing said polymeric micellar nanoparticle.

41. (canceled)

42. (canceled)

43. A diagnostic pharmaceutical composition comprising the polymeric micellar nanoparticle of claim 1 and a pharmaceutically acceptable excipient.

44. A kit comprising an instruction and the polymeric micellar nanoparticle of claim 1.
